SEQUENCE LISTING (519 sequences)

SEQ ID NO: 1 - protein, 188 aa, Homo sapiens [sequence omitted]

SEQ ID NO: 2 - protein, 189 aa, Homo sapiens [sequence omitted]

SEQ ID NO: 3 - protein, 10 aa, synthetic construct
KLVVVGAGGV

SEQ ID NO: 4 - protein, 9 aa, synthetic construct
KLVVGAGGV

SEQ ID NO: 5 - protein, 9 aa, synthetic construct
  VARIANT 7: note = where X (corresponding to G12 of wild-type KRAS) is an amino acid other than Gly
KLVVGAXGV

SEQ ID NO: 6 - protein, 9 aa, synthetic construct
KLVVGADGV

SEQ ID NO: 7 - protein, 9 aa, synthetic construct
KLVVGAVGV

SEQ ID NO: 8 - protein, 9 aa, synthetic construct
  VARIANT 8: note = where X is Cys, Asp, Arg, Val, Ser, or Ala
KLVVGAGXV

SEQ ID NO: 9 - protein, 9 aa, synthetic construct
KLVVVAGGV

SEQ ID NO: 10 - protein, 9 aa, synthetic construct
  VARIANT 7: note = where X (corresponding to G12 of wild-type KRAS) is an amino acid other than Gly
KLVVVAXGV

SEQ ID NO: 11 - protein, 9 aa, synthetic construct
KLVVVADGV

SEQ ID NO: 12 - protein, 9 aa, synthetic construct
KLVVVAVGV

SEQ ID NO: 13 - protein, 9 aa, synthetic construct
  VARIANT 8: note = where X (corresponding to G13 of wild-type KRAS) is an amino acid other than Gly
KLVVVAGXV

SEQ ID NO: 14-41 - skipped (28 entries)

SEQ ID NO: 42 - protein, 99 aa, Homo sapiens [sequence omitted]

SEQ ID NO: 43 - protein, 99 aa, synthetic construct
IQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 44 - protein, 275 aa, Homo sapiens [sequence omitted]

SEQ ID NO: 45 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 46 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 47 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGYYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 48 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGAYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPCGDGTFQKWAAVVVPSGQEQRYTCHVQHEGLPKPLTLRWE

SEQ ID NO: 49 - protein, 275 aa, Homo sapiens
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 50 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 51 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 52 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 53 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 54 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 55 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 56 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 57 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 58 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRAYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 59 - protein, 275 aa, Homo sapiens
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMAAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 60 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 61 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 62 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGYYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 63 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGAYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPCGDGTFQKWASVVVPSGQEQRYTCHVQHEGLPKPLTLRWE

SEQ ID NO: 64 - protein, 275 aa, Homo sapiens
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 65 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 66 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 67 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 68 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWE

SEQ ID NO: 69 - protein, 275 aa, Homo sapiens
GSHSMRYFSTSVSRPGRGEPRFIAVGYVDDTQFVRFDSDAASQRMEPRAPWIEQEGPEYWDEETGKVKAHSQTDRENLRIALRYYNQSEAGSHTLQMMFGCDVGSDGRFLRGYHQYAYDGKDYIALKEDLRSWTAADMAAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 70 - protein, 275 aa, Homo sapiens; identical to SEQ ID NO: 54

SEQ ID NO: 71 - protein, 275 aa, Homo sapiens
GSHSMRYFSTSVSRPGRGEPRFIAVGYVDDTQFVRFDSDAASQRMEPRAPWIEQEGPEYWDEETGKVKAQSQTDRENLRIALRYYNQSEAGSHTLQMMFGCDVGSDGRFLRGYHQYAYDGKDYIALKEDLRSWTAADMAAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 72 - protein, 275 aa, Homo sapiens
GSHSMRYFFTSVSRPGRGEPRFIAVGYVDDTQFVRFDSDAASQKMEPRAPWIEQEGPEYWDQETRNMKAHSQTDRANLGTLRGYYNQSEDGSHTIQIMYGCDVGPDGRFLRGYRQDAYDGKDYIALNEDLRSWTAADMAAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 73 - protein, 275 aa, Homo sapiens; identical to SEQ ID NO: 64

SEQ ID NO: 74 - protein, 275 aa, Homo sapiens; identical to SEQ ID NO: 49

SEQ ID NO: 75 - protein, 275 aa, Homo sapiens [sequence omitted]

SEQ ID NO: 76 - protein, 275 aa, Homo sapiens; identical to SEQ ID NO: 59

SEQ ID NO: 77 - protein, 275 aa, Homo sapiens
GSHSMRYFYTSVSRPGRGEPRFIAVGYVDDTQFVRFDSDAASQRMEPRAPWIEQEGPEYWDRNTRKVKAQSQTDRVDLGTLRGYYNQSEDGSHTIQRMYGCDVGPDGRFLRGYQQDAYDGKDYIALNEDLRSWTAADMAAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 78 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRXYNQSEAGSHTLQMMFGCDVGSDGRFLRGYHQYAYDGKDYIALKEDLRSWTAADMCAQITQRKWEAARVAEQLRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 79 - protein, 275 aa, synthetic construct
GSHSMRYFSTSVSRPGRGEPRFIAVGYVDDTQFVRFDSDAASQRMEPRAPWIEQEGPEYWDEETGKVKAHSQTDRENLRIALRXYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 80 - protein, 275 aa, synthetic construct
GSHSMRYFSTSVSRPGRGEPRFIAVGYVDDTQFVRFDSDAASQRMEPRAPWIEQEGPEYWDEETGKVKAQSQTDRENLRIALRXYNQSEAGSHTLQMMFGCDVGSDGRFLRGYHQYAYDGKDYIALKEDLRSWTAADMCAQITKRKWEAAHVAEQQRAYLEGTCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 81 - protein, 275 aa, synthetic construct
GSHSMRYFFTSVSRPGRGEPRFIAVGYVDDTQFVRFDSDAASQKMEPRAPWIEQEGPEYWDQETRNMKAHSQTDRANLGTLRGXYNQSEDGSHTIQIMYGCDVGPDGRFLRGYRQDAYDGKDYIALNEDLRSWTAADMCAQITKRKWEAVHAAEQRRVYLEGRCVDGLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 82 - protein, 275 aa, synthetic construct
GSHSMRYFFTSVSRPGRGEPRFIAVGYVDDTQFVRFDSDAASQRMEPRAPWIEQEGPEYWDQETRNVKAQSQTDRVDLGTLRGXYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 83 - protein, 275 aa, synthetic construct
GSHSMRYFYTSVSRPGRGEPRFIAVGYVDDTQFVRFDSDAASQRMEPRAPWIEQEGPEYWDQETRNVKAQSQTDRVDLGTLRGXYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPAGDGTFQKWAAVVVPSGEEQRYTCHVQHEGLPKPLTLRWE

SEQ ID NO: 84 - protein, 275 aa, synthetic construct
GSHSMRYFFTSVSRPGRGEPRFIAVGYVDDTQFVRFDSDAASQRMEPRAPWIEQEGPEYWDGETRKVKAHSQTHRVDLGTLRGXYNQSEAGSHTVQRMYGCDVGSDWRFLRGYHQYAYDGKDYIALKEDLRSWTAADMCAQTTKHKWEAAHVAEQLRAYLEGTCVEWLRRYLENGKETLQRTDAPKTHMTHHAVSDHEATLRCWALSFYPAEITLTWQRDGEDQTQDTELVETRPAGDGTFQKWAAVVVPSGQEQRYTCHVQHEGLPKPLTLRWE

SEQ ID NO: 85 - protein, 275 aa, synthetic construct
GSHSMRYFTTSVSRPGRGEPRFIAVGYVDDTQFVRFDSDAASQRMEPRAPWIEQEGPEYWDRNTRNVKAHSQIDRVDLGTLRGXYNQSEAGSHTIQMMYGCDVGSDGRFLRGYQQDAYDGKDYIALNEDLRSWTAADMCAQITQRKWEAARVAEQLRAYLEGTCVEWLRRYLENGKETLQRTDPPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 86 - protein, 275 aa, synthetic construct
GSHSMRYFYTSVSRPGRGEPRFIAVGYVDDTQFVRFDSDAASQRMEPRAPWIEQEGPEYWDRNTRKVKAQSQTDRVDLGTLRGXYNQSEDGSHTIQRMYGCDVGPDGRFLRGYQQDAYDGKDYIALNEDLRSWTAADMCAQITQRKWETAHEAEQWRAYLEGTCVEWLRRYLENGKETLQRTDAPKTHMTHHAVSDHEATLRCWALSFYPAEITLTWQRDGEDQTQDTELVETRPAGDGTFQKWASVVVPSGQEQRYTCHVQHEGLPKPLTLRWE

SEQ ID NO: 87 - protein, 276 aa, Homo sapiens
GSHSMRYFYTSVSRPGRGEPRFISVGYVDDTQFVRFDSDAASPREEPRAPWIEQEGPEYWDRNTQICKTNTQTYRENLRTALRYYNQSEAGSHTLQRMYGCDVGPDGRLLRGHNQFAYDGKDYIALNEDLSSWTAADTAAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 88 - protein, 276 aa, Homo sapiens
GSHSMRYFYTAMSRPGRGEPRFIAVGYVDDTQFVRFDSDAASPRTEPRAPWIEQEGPEYWDRNTQIFKTNTQTYRENLRIALRYYNQSEAGSHIIQRMYGCDLGPDGRLLRGHDQSAYDGKDYIALNEDLSSWTAADTAAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 89 - protein, 275 aa, Homo sapiens
GSHSMRYFYTAMSRPGRGEPRFIAVGYVDDTQFVRFDSDAASPRMAPRAPWIEQEGPEYWDRNTQISKTNTQTYRESLRNLRGYYNQSEAGSHIIQRMYGCDVGPDGRLLRGYDQSAYDGKDYIALNEDLSSWTAADTAAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVWPSGEEQRYTCHVQHEGLPKPLTLRWEP

SEQ ID NO: 90 - protein, 276 aa, Homo sapiens
GSHSMRYFYTAMSRPGRGEPRFIAVGYVDDTQFVRFDSDAASPRMAPRAPWIEQEGPEYWDRETQKYKRQAQTDRVSLRNLRGYYNQSEAGSHTLQRMYGCDVGPDGRLLRGHDQSAYDGKDYIALNEDLSSWTAADTAAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 91 - protein, 276 aa, Homo sapiens
GSHSMRYFHTAMSRPGRGEPRFITVGYVDDTLFVRFDSDATSPRKEPRAPWIEQEGPEYWDRETQISKTNTQTYRESLRNLRGYYNQSEAGSHTLQRMYGCDVGPDGRLLRGHNQYAYDGKDYIALNEDLRSWTAADTAAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 92 - protein, 276 aa, Homo sapiens [sequence omitted]

SEQ ID NO: 93 - protein, 276 aa, Homo sapiens
GSHSMRYFDTAMSRPGRGEPRFISVGYVDDTQFVRFDSDAASPREEPRAPWIEQEGPEYWDRNTQIFKTNTQTDRESLRNLRGYYNQSEAGSHTLQSMYGCDVGPDGRLLRGHNQYAYDGKDYIALNEDLRSWTAADTAAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 94 - protein, 276 aa, synthetic construct
GSHSMRYFYTSVSRPGRGEPRFISVGYVDDTQFVRFDSDAASPREEPRAPWIEQEGPEYWDRNTQICKTNTQTYRENLRTALRXYNQSEAGSHTLQRMYGCDVGPDGRLLRGHNQFAYDGKDYIALNEDLSSWTAADTCAQITQRKWEAARVAEQLRTYLEGTCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 95 - protein, 276 aa, synthetic construct
GSHSMRYFYTAMSRPGRGEPRFIAVGYVDDTQFVRFDSDAASPRTEPRAPWIEQEGPEYWDRNTQIFKTNTQTYRENLRIALRXYNQSEAGSHIIQRMYGCDLGPDGRLLRGHDQSAYDGKDYIALNEDLSSWTAADTCAQITQRKWEAARVAEQLRAYLEGLCVEWLRRYLENGKETLQRADPPKTHVTHHPVSDHEATLRCWALGFYPAEITLTWQRDGEDQTQDTELVETRPAGDRTFQKWAAVVVPSGEEQRYTCHVQHEGLPKPLTLRWEP

SEQ ID NO: 96 - protein, 275 aa, synthetic construct
GSHSMRYFYTAMSRPGRGEPRFIAVGYVDDTQFVRFDSDAASPRMAPRAPWIEQEGPEYWDRNTQISKTNTQTYRESLRNLRGXYNQSEAGSHIIQRMYGCDVGPDGRLLRGYDQSAYDGKDYIALNEDLSSWTAADTCAQITQRKWEAAREAEQLRAYLEGLCVEWLRRYLENGKETLQRADPPKTHVTHHPISDHEATLRCWALGFYPAEITLTWQRDGEDQTQDTELVETRPAGDRTFQKWAAVWPSGEEQRYTCHVQHEGLPKPLTLRWEP

SEQ ID NO: 97 - protein, 276 aa, synthetic construct
GSHSMRYFYTAMSRPGRGEPRFIAVGYVDDTQFVRFDSDAASPRMAPRAPWIEQEGPEYWDRETQKYKRQAQTDRVSLRNLRGXYNQSEAGSHTLQRMYGCDVGPDGRLLRGHDQSAYDGKDYIALNEDLSSWTAADTCAQITQRKWEAAREAEQWRAYLEGLCVEWLRRYLENGKETLQRADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 98 - protein, 276 aa, synthetic construct
GSHSMRYFHTAMSRPGRGEPRFITVGYVDDTLFVRFDSDATSPRKEPRAPWIEQEGPEYWDRETQISKTNTQTYRESLRNLRGXYNQSEAGSHTLQRMYGCDVGPDGRLLRGHNQYAYDGKDYIALNEDLRSWTAADTCAQISQRKLEAARVAEQLRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 99 - protein, 276 aa, synthetic construct
GSHSMRYFYTSVSRPGRGEPRFISVGYVDDTQFVRFDSDAASPREEPRAPWIEQEGPEYWDRNTQIYKAQAQTDRESLRNLRGXYNQSEAGSHTLQSMYGCDVGPDGRLLRGHDQYAYDGKDYIALNEDLRSWTAADTCAQITQRKWEAAREAEQRRAYLEGECVEWLRRYLENGKDKLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 100 - protein, 276 aa, synthetic construct
GSHSMRYFDTAMSRPGRGEPRFISVGYVDDTQFVRFDSDAASPREEPRAPWIEQEGPEYWDRNTQIFKTNTQTDRESLRNLRGXYNQSEAGSHTLQSMYGCDVGPDGRLLRGHNQYAYDGKDYIALNEDLRSWTAADTCAQITQRKWEAARVAEQDRAYLEGTCVEWLRRYLENGKDTLERADPPKTHVTHHPISDHEATLRCWALGFYPAEITLTWQRDGEDQTQDTELVETRPAGDRTFQKWAAVVVPSGEEQRYTCHVQHEGLPKPLTLRWEP

SEQ ID NO: 101 - protein, 275 aa, Homo sapiens
CSHSMRYFDTAVSRPGRGEPRFISVGYVDDTQFVRFDSDAASPRGEPRAPWVEQEGPEYWDRETQNYKRQAQADRVSLRNLRGYYNQSEDGSHTLQRMYGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 102 - protein, 275 aa, Homo sapiens
CSHSMRYFDTAVSRPGRGEPRFISVGYVDDTQFVRFDSDAASPRGEPRAPWVEQEGPEYWDRETQKYKRQAQADRVSLRNLRGYYNQSEDGSHTLQRMSGCDLGPDGRLLRGYDQSAYDGKDYIALNEDLRSWTAADTAAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 103 - protein, 275 aa, Homo sapiens
GSHSMRYFSTSVSWPGRGEPRFIAVGYVDDTQFVRFDSDAASPRGEPREPWVEQEGPEYWDRETQKYKRQAQADRVNLRKLRGYYNQSEDGSHTLQRMFGCDLGPDGRLLRGYNQFAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWK

SEQ ID NO: 104 - protein, 275 aa, Homo sapiens
CSHSMKYFFTSVSRPGRGEPRFISVGYVDDTQFVRFDSDAASPRGEPRAPWVEQEGPEYWDRETQKYKRQAQTDRVSLRNLRGYYNQSEAGSHTLQWMCGCDLGPDGRLLRGYDQYAYDGKDYIALNEDLRSWTAADTAAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWE

SEQ ID NO: 105 - protein, 275 aa, Homo sapiens
CSHSMRYFDTAVSRPGRGEPRFISVGYVDDTQFVRFDSDAASPRGEPRAPWVEQEGPEYWDRETQKYKRQAQADRVNLRKLRGYYNQSEDGSHTLQWMYGCDLGPDGRLLRGYDQSAYDGKDYIALNEDLRSWTAADTAAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 106 - protein, 275 aa, Homo sapiens
CSHSMRYFYTAVSRPGRGEPRFIAVGYVDDTQFVQFDSDAASPRGEPRAPWVEQEGPEYWDRETQKYKRQAQTDRVSLRNLRGYYNQSEAGSHTLQRMYGCDLGPDGRLLRGYNQFAYDGKDYIALNEDLRSWTAADTAAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWG

SEQ ID NO: 107 - protein, 275 aa, Homo sapiens
CSHSMRYFYTAVSRPGRGEPHFIAVGYVDDTQFVRFDSDAASPRGEPRAPWVEQEGPEYWDRETQNYKRQAQTDRVNLRKLRGYYNQSEAGSHIIQRMYGCDLGPDGRLLRGHDQLAYDGKDYIALNEDLRSWTAADTAAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 108 - protein, 275 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEARSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 109 - protein, 275 aa, Homo sapiens
GSHSMRYFYTAVSRPGRGEPHFIAVGYVDDTQFVRFDSDAASPRGEPRAPWVEQEGPEYWDRETQKYKRQAQTDRVSLRNLRGYYNQSEAGSHIIQRMYGCDVGPDGRLLRGYDQYAYDGKDYIALNEDLRSWTAADTAAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 110 - protein, 275 aa, synthetic construct
CSHSMRYFDTAVSRPGRGEPRFISVGYVDDTQFVRFDSDAASPRGEPRAPWVEQEGPEYWDRETQNYKRQAQADRVSLRNLRGXYNQSEDGSHTLQRMYGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 111 - protein, 275 aa, synthetic construct
CSHSMRYFDTAVSRPGRGEPRFISVGYVDDTQFVRFDSDAASPRGEPRAPWVEQEGPEYWDRETQKYKRQAQADRVSLRNLRGXYNQSEDGSHTLQRMSGCDLGPDGRLLRGYDQSAYDGKDYIALNEDLRSWTAADTCAQITQRKLEAARAAEQLRAYLEGTCVEWLRRYLENGKETLQRAEPPKTHVTHHPLSDHEATLRCWALGFYPAEITLTWQRDGEDQTQDTELVETRPAGDGTFQKWAAVVVPSGQEQRYTCHMQHEGLQEPLTLSWE

SEQ ID NO: 112 - protein, 275 aa, synthetic construct
GSHSMRYFSTSVSWPGRGEPRFIAVGYVDDTQFVRFDSDAASPRGEPREPWVEQEGPEYWDRETQKYKRQAQADRVNLRKLRGXYNQSEDGSHTLQRMFGCDLGPDGRLLRGYNQFAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWK

SEQ ID NO: 113 - protein, 275 aa, synthetic construct
CSHSMKYFFTSVSRPGRGEPRFISVGYVDDTQFVRFDSDAASPRGEPRAPWVEQEGPEYWDRETQKYKRQAQTDRVSLRNLRGXYNQSEAGSHTLQWMCGCDLGPDGRLLRGYDQYAYDGKDYIALNEDLRSWTAADTCAQITQRKWEAAREAEQRRAYLEGTCVEWLRRYLENGKETLQRAEHPKTHVTHHPVSDHEATLRCWALGFYPAEITLTWQWDGEDQTQDTELVETRPAGDGTFQKWAAVMVPSGEEQRYTCHVQHEGLPEPLTLRWE

SEQ ID NO: 114 - protein, 275 aa, synthetic construct
CSHSMRYFDTAVSRPGRGEPRFISVGYVDDTQFVRFDSDAASPRGEPRAPWVEQEGPEYWDRETQKYKRQAQADRVNLRKLRGXYNQSEDGSHTLQWMYGCDLGPDGRLLRGYDQSAYDGKDYIALNEDLRSWTAADTCAQITQRKWEAAREAEQWRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 115 - protein, 275 aa, synthetic construct
CSHSMRYFYTAVSRPGRGEPRFIAVGYVDDTQFVQFDSDAASPRGEPRAPWVEQEGPEYWDRETQKYKRQAQTDRVSLRNLRGXYNQSEAGSHTLQRMYGCDLGPDGRLLRGYNQFAYDGKDYIALNEDLRSWTAADTCAQITQRKWEAARTAEQLRAYLEGTCVEWLRRYLENGKKTLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWG

SEQ ID NO: 116 - protein, 275 aa, synthetic construct
CSHSMRYFYTAVSRPGRGEPHFIAVGYVDDTQFVRFDSDAASPRGEPRAPWVEQEGPEYWDRETQNYKRQAQTDRVNLRKLRGXYNQSEAGSHIIQRMYGCDLGPDGRLLRGHDQLAYDGKDYIALNEDLRSWTAADTCAQITQRKWEAAREAEQLRAYLEGTCVEWLRRYLENGKETLQRAEHPKTHVTHHPVSDHEATLRCWALGFYPAEITLTWQRDGEDQTQDTELVETRPAGDGTFQKWAAVVVPSGEEQRYTCHVQHEGLPEPLTLRWE

SEQ ID NO: 117 - protein, 275 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGXYNQSEARSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 118 - protein, 275 aa, synthetic construct
GSHSMRYFYTAVSRPGRGEPHFIAVGYVDDTQFVRFDSDAASPRGEPRAPWVEQEGPEYWDRETQKYKRQAQTDRVSLRNLRGXYNQSEAGSHIIQRMYGCDVGPDGRLLRGYDQYAYDGKDYIALNEDLRSWTAADTCAQITQRKWEAAREAEQLRAYLEGLCVEWLRRYLKNGKETLQRAEHPKTHVTHHPVSDHEATLRCWALGFYPAEITLTWQWDGEDQTQDTELVETRPAGDGTFQKWAAVVVPSGEEQRYTCHVQHEGLPEPLTLRWE

SEQ ID NO: 119 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 120 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 121 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 122 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 123 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDRRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 124 - protein, 275 aa, Homo sapiens
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTAAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 125 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 126 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPAGDGTFQKWAAVVVPSGEEQRYTCHVQHEGLPEPVTLRWK

SEQ ID NO: 127 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGYYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 128 - protein, 275 aa, synthetic construct
GSHSLKYFHTSVSRPGRGEPRFISVGYVDDTQFVRFDNDAASPRMVPRAPWMEQEGSEYWDRETRSARDTAQIFRVNLRTLRGAYNQSEAGSHTLQWMHGCELGPDGRFLRGYEQFAYDGKDYLTLNEDLRSWTAVDTCAQISEQKSNDASEAEHQRAYLEDTCVEWLHKYLEKGKETLLHLEPPKTHVTHHPISDHEATLRCWALGFYPAEITLTWQQDGEGHTQDTELVETRPCGDGTFQKWAAVVVPSGEEQRYTCHVQHEGLPEPVTLRWK

SEQ ID NO: 129 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLLRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 130 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGAYNQSEASSHTLQWMIGCDLGSDGRLLRGYEQYAYDGKDYLALNEDLRSWTAADTCAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 131 - protein, 275 aa, Homo sapiens
GSHSMRYFSAAVSRPGRGEPRFIAMGYVDDTQFVRFDSDSACPRMEPRAPWVEQEGPEYWEEETRNTKAHAQTDRMNLQTLRGYYNQSEASSHTLQWMIGCDLGSDGRLIRGYEQYAYDGKDYLALNEDLRSWTAADTAAQISKRKCEAANVAEQRRAYLEGTCVEWLHRYLENGKEMLQRADPPKTHVTHHPVFDYEATLRCWALGFYPAEIILTWQRDGEDQTQDVELVETRPAGDGTFQKWAAVVVPSGEEQRYTCHVQHEGLPEPLMLRWK

SEQ ID NO: 132 - protein, 275 aa, synthetic construct
GSHSMRYFSAAVSRPGRGEPRFIAMGYVDDTQFVRFDSDSACPRMEPRAPWVEQEGPEYWEEETRNTKAHAQTDRMNLQTLRGAYNQSEASSHTLQWMIGCDLGSDGRLIRGYEQYAYDGKDYLALNEDLRSWTAADTCAQISKRKCEAANVAEQRRAYLEGTCVEWLHRYLENGKEMLQRADPPKTHVTHHPVFDYEATLRCWALGFYPAEIILTWQRDGEDQTQDVELVETRPCGDGTFQKWAAVVVPSGEEQRYTCHVQHEGLPEPLMLRWK

SEQ ID NO: 133 - protein, 227 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 134 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 135 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 136 - protein, 227 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 137 - protein, 227 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 138 - protein, 227 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 139 - protein, 226 aa, Homo sapiens
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 140 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 141 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 142 - protein, 226 aa, synthetic construct
DKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 143 - protein, 226 aa, synthetic construct
DKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 144 - protein, 226 aa, synthetic construct
DKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 145 - protein, 272 aa, Homo sapiens
MDSYLLMWGLLTFIMVPGCQAELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTGEMETSQFPGEEKPQASPEGRPESETSCLVTTTDFQIQTEMAATMETSIFTTEYQVAVAGCVFLLISVLLLSGLTWQRRQRKSRRTI

SEQ ID NO: 146 - protein, 551 aa, Homo sapiens
MAAPALSWRLPLLILLLPLATSWASAAVNGTSQFTCFYNSRANISCVWSQDGALQDTSCQVHAWPDRRRWNQTCELLPVSQASWACNLILGAPDSQKLTTVDIVTLRVLCREGVRWRVMAIQDFKPFENLRLMAPISLQVVHVETHRCNISWEISQASHYFERHLEFEARTLSPGHTWEEAPLLTLKQKQEWICLETLTPDTQYEFQVRVKPLQGEFTTWSPWSQPLAFRTKPAALGKDTIPWLGHLLVGLSGAFGFIILVYLLINCRNTGPWLKKVLKCNTPDPSKFFSQLSSEHGGDVQKWLSSPFPSSSFSPGGLAPEISPLEVLERDKVTQLLLQQDKVPEPASLSSNHSLTSCFTNQGYFFFHLPDALEIEACQVYFTYDPYSEEDPDEGVAGAPTGSSPQPLQPLSGEDDAYCTFPSRDDLLLFSPSLLGGPSPPSTAPGGSGAGEERMPPSLQERVPRDWDPQPLGPPTPGVPDLVDFQPPPELVLREAGEEVPDAGPREGVSFPWSRPPGQGEFRALNARLPLNTDAYLSLQELQGQDPTHLV

SEQ ID NO: 147 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

SEQ ID NO: 148 - protein, 133 aa, Homo sapiens [sequence omitted]

SEQ ID NO: 149 - protein, 930 aa, synthetic construct
XGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 150 - protein, 930 aa, synthetic construct
XGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 151 - protein, 939 aa, synthetic construct
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 152 - protein, 939 aa, synthetic construct
VVVGAVGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGAYNQSEAGSHTIQIMYGCDVGSDGRFLRGYRQDAYDGKDYIALNEDLRSWTAADMCAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 153 - protein, 938 aa, synthetic construct
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGAYNQSEDGSHTIQIMYGCDVGPDGRFLRGYRQDAYDGKDYIALNEDLRSWTAADMCAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTL

SEQ ID NO: 154 - protein, 15 aa, synthetic construct
GCGGSGGGGSGGGGS

SEQ ID NO: 155 - protein, 15 aa, synthetic construct
GGGGSGGGGSGGGGS

SEQ ID NO: 156 - protein, 5 aa, synthetic construct
AAAGG

SEQ ID NO: 157 - protein, 8 aa, synthetic construct
APAPAPAP

SEQ ID NO: 158 - protein, 20 aa, synthetic construct
GGGGSGGGGSGGGGSGGGGS

SEQ ID NO: 159 - protein, 13 aa, synthetic construct
GCGGSGGGSGGGS

SEQ ID NO: 160 - protein, 15 aa, synthetic construct
GGGGSGGGGSGGGGS

SEQ ID NO: 161 - protein, 20 aa, synthetic construct
GGGGSGGGGSGGGGSGGGGS

SEQ ID NO: 162 - protein, 10 aa, synthetic construct
VVVGAVGVGK

SEQ ID NO: 163 - protein, 10 aa, synthetic construct
VVVGADGVGK

SEQ ID NO: 164 - protein, 939 aa, synthetic construct
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 165 - protein, 939 aa, synthetic construct
VVVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFFTSVSRPGRGEPRFIAVGYVDDTQFVRFDSDAASQRMEPRAPWIEQEGPEYWDQETRNVKAQSQTDRVDLGTLRGYYNQSEAGSHTIQIMYGCDVGSDGRFLRGYRQDAYDGKDYIALNEDLRSWTAADMAAQITKRKWEAAHEAEQLRAYLDGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 166 - protein, 939 aa, synthetic construct
VVVGAVGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 167 - protein, 939 aa, synthetic construct
VVVGADGVGKGGGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDMGGGGSGGGGSGGGGSGSHSMRYFYTSVSRPGRGEPRFIAVGYVDDTQFVRFDSDAASQRMEPRAPWIEQEGPEYWDQETRNVKAQSQTDRVDLGTLRGYYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGAPAPAPAPAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 168 - protein, 800 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTGGGGSGGGGSGGGGSGGGGSAPTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTAPAPAPAPGSHSMRYFYTSVSRPGRGEPRFIAVGYVDDTQFVRFDSDAASQRMEPRAPWIEQEGPEYWDQETRNVKAQSQTDRVDLGTLRGCYNQSEDGSHTIQIMYGCDVGPDGRFLRGYRQDAYDGKDYIALNEDLRSWTAADMAAQITKRKWEAAHAAEQQRAYLEGTCVEWLRRYLENGKETLQRTDPPKTHMTHHPISDHEATLRCWALGFYPAEITLTWQRDGEDQTQDTELVETRPCGDGTFQKWAAVVVPSGEEQRYTCHVQHEGLPKPLTLRWEAAAGGDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 169 - protein, 124 aa, synthetic construct
VVVGADGVGKGCGGSGGGGSGGGGSIQRTPKIQVYSCHPAENGKSNFLNCYVSGFHPSDIEVDLLKNGERIEKVEHSDLSFSKDWSFYLLYYTEFTPTEKDEYACRVNHVTLSQPKIVKWDRDM

SEQ ID NO: 170 - protein, 10 aa, synthetic construct
VVVGAVGVGK

SEQ ID NO: 171-195 - skipped (25 entries)

SEQ ID NO: 196 - protein, 9 aa, synthetic construct
CADMAAQTT

SEQ ID NO: 197 - protein, 9 aa, synthetic construct
ACDMAAQTT

SEQ ID NO: 198 - protein, 9 aa, synthetic construct
AACMAAQTT

SEQ ID NO: 199 - protein, 9 aa, synthetic construct
AADCAAQTT

SEQ ID NO: 200 - protein, 9 aa, synthetic construct
AADMCAQTT

SEQ ID NO: 201 - protein, 9 aa, synthetic construct
AADMACQTT

SEQ ID NO: 202 - protein, 9 aa, synthetic construct
AADMAACTT

SEQ ID NO: 203 - protein, 9 aa, synthetic construct
AADMAAQCT

SEQ ID NO: 204 - protein, 9 aa, synthetic construct
AADMAAQTC

SEQ ID NO: 205 - protein, 9 aa, synthetic construct
CADMAAQIT

SEQ ID NO: 206 - protein, 9 aa, synthetic construct
ACDMAAQIT

SEQ ID NO: 207 - protein, 9 aa, synthetic construct
AACMAAQIT

SEQ ID NO: 208 - protein, 9 aa, synthetic construct
AADCAAQIT

SEQ ID NO: 209 - protein, 9 aa, synthetic construct
AADMCAQIT

SEQ ID NO: 210 - protein, 9 aa, synthetic construct
AADMACQIT

SEQ ID NO: 211 - protein, 9 aa, synthetic construct
AADMAACIT

SEQ ID NO: 212 - protein, 9 aa, synthetic construct
AADMAAQCT

SEQ ID NO: 213 - protein, 9 aa, synthetic construct
AADMAAQIC

SEQ ID NO: 214 - protein, 9 aa, synthetic construct
CADTAAQIT

SEQ ID NO: 215 - protein, 9 aa, synthetic construct
ACDTAAQIT

SEQ ID NO: 216 - protein, 9 aa, synthetic construct
AACTAAQIT

SEQ ID NO: 217 - protein, 9 aa, synthetic construct
AADCAAQIT

SEQ ID NO: 218 - protein, 9 aa, synthetic construct
AADTCAQIT

SEQ ID NO: 219 - protein, 9 aa, synthetic construct
AADTACQIT

SEQ ID NO: 220 - protein, 9 aa, synthetic construct
AADTAACIT

SEQ ID NO: 221 - protein, 9 aa, synthetic construct
AADTAAQCT

SEQ ID NO: 222 - protein, 9 aa, synthetic construct
AADTAAQIC

SEQ ID NO: 223 - protein, 9 aa, synthetic construct
CVDTAAQIS

SEQ ID NO: 224 - protein, 9 aa, synthetic construct
ACDTAAQIS

SEQ ID NO: 225 - protein, 9 aa, synthetic construct
AVCTAAQIS

SEQ ID NO: 226 - protein, 9 aa, synthetic construct
AVDCAAQIS

SEQ ID NO: 227 - protein, 9 aa, synthetic construct
AVDTCAQIS

SEQ ID NO: 228 - protein, 9 aa, synthetic construct
AVDTACQIS

SEQ ID NO: 229 - protein, 9 aa, synthetic construct
AVDTAACIS

SEQ ID NO: 230 - protein, 9 aa, synthetic construct
AVDTAAQCS

SEQ ID NO: 231 - protein, 9 aa, synthetic construct
AVDTAAQIC

SEQ ID NO: 232 - protein, 55 aa, synthetic construct
  VARIANT 6..55: note = Wherein up to 9 copies of GGGGS may be deleted
CGGGSGGGGSGGGGSGGGGSGGGGSGGGGSGGGGSGGGGSGGGGSGGGGSGGGGS

SEQ ID NO: 233 - protein, 55 aa, synthetic construct
  VARIANT 6..55: note = Wherein up to 9 copies of GGGGS may be deleted
GCGGSGGGGSGGGGSGGGGSGGGGSGGGGSGGGGSGGGGSGGGGSGGGGSGGGGS

SEQ ID NO: 234 - protein, 55 aa, synthetic construct
  VARIANT 6..55: note = Wherein up to 9 copies of GGGGS may be deleted
GGCGSGGGGSGGGGSGGGGSGGGGSGGGGSGGGGSGGGGSGGGGSGGGGSGGGGS

SEQ ID NO: 235 - protein, 10 aa, synthetic construct
CGGGSGGGGS

SEQ ID NO: 236 - protein, 15 aa, synthetic construct
CGGGSGGGGSGGGGS

SEQ ID NO: 237 - protein, 20 aa, synthetic construct
CGGGSGGGGSGGGGSGGGGS

SEQ ID NO: 238 - protein, 10 aa, synthetic construct
GCGGSGGGGS

SEQ ID NO: 239 - protein, 15 aa, synthetic construct
GCGGSGGGGSGGGGS

SEQ ID NO: 240 - protein, 20 aa, synthetic construct
GCGGSGGGGSGGGGSGGGGS

SEQ ID NO: 241 - protein, 10 aa, synthetic construct
GGCGSGGGGS

SEQ ID NO: 242 - protein, 15 aa, synthetic construct
GGCGSGGGGSGGGGS

SEQ ID NO: 243 - protein, 20 aa, synthetic construct
GGCGSGGGGSGGGGSGGGGS

SEQ ID NO: 244-324 - skipped (81 entries)

SEQ ID NO: 325 - protein, 5 aa, synthetic construct
  VARIANT >5: note = Wherein the entire sequence of amino acids 1-5 may be repeated one or more times
GSGGS

SEQ ID NO: 326 - protein, 5 aa, synthetic construct
  VARIANT >5: note = Wherein the entire sequence of amino acids 1-5 may be repeated one or more times
GGGGS

SEQ ID NO: 327 - protein, 4 aa, synthetic construct
  VARIANT >4: note = Wherein the entire sequence of amino acids 1-4 may be repeated one or more times
GGGS

SEQ ID NO: 328 - protein, 50 aa, synthetic construct
  VARIANT 6..50: note = Wherein up to 9 copies of GGGGS may be deleted
GGGGSGGGGSGGGGSGGGGSGGGGSGGGGSGGGGSGGGGSGGGGSGGGGS

SEQ ID NO: 329 - protein, 10 aa, synthetic construct
GGGGSGGGGS

SEQ ID NO: 330 - protein, 15 aa, synthetic construct
GGGGSGGGGSGGGGS

SEQ ID NO: 331 - protein, 20 aa, synthetic construct
GGGGSGGGGSGGGGSGGGGS

SEQ ID NO: 332 - protein, 35 aa, synthetic construct
GGGGSGGGGSGGGGSGGGGSGGGGSGGGGSGGGGS

SEQ ID NO: 333 - protein, 5 aa, synthetic construct
AAAGG

SEQ ID NO: 334 - protein, 133 aa, synthetic construct
  VARIANT 16: note = >Wherein X is any amino acid other than His
  VARIANT 42: note = Wherein X is any amino acid other than Phe
APTSSSTKKTQLQLEXLLLDLQMILNGINNYKNPKLTRMLTXKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 335 - protein, 133 aa, synthetic construct; identical to SEQ ID NO: 334
  VARIANT 16: note = Wherein X is is Ala, Arg, Asn, Asp, Cys, Gln, Glu, Gly, Ile, Leu, Lys, Met, Phe, Pro, Ser, Thr, Trp, Tyr, or Val
  VARIANT 42: note = Wherein X is Ala, Arg, Asn, Asp, Cys, Gln, Glu, Gly, His, Ile, Leu, Lys, Met, Pro, Ser, Thr, Trp, Tyr, or Val

SEQ ID NO: 336 - protein, 133 aa, synthetic construct
APTSSSTKKTQLQLEALLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 337 - protein, 133 aa, synthetic construct; identical to SEQ ID NO: 336

SEQ ID NO: 338 - protein, 133 aa, synthetic construct
APTSSSTKKTQLQLETLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 339-340 - skipped (2 entries)

SEQ ID NO: 341 - protein, 219 aa, synthetic construct
FTVTVPKDLYVVEYGSNMTIECKFPVEKQLDLAALIVYWEMEDKNIIQFVHGEEDLKVQHSSYRQRARLLKDQLSLGNAALQITDVKLQDAGVYRCMISYGGADYKRITVKVNAPYNKINQRILVVDPVTSEHELTCQAEGYPKAEVIWTSSDHQVLSGKTTTTNSKREEKLFNVTSTLRINTTTNEIFYCTFRRLDPEENHTAELVIPGNILNVSIKI

SEQ ID NO: 342 - protein, 167 aa, synthetic construct
DPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPA

SEQ ID NO: 343 - protein, 240 aa, synthetic construct
QEKEVRAMVGSDVELSCACPEGSRFDLNDVYVYWQTSESKTVVTYHIPQNSSLENVDSRYRNRALMSPAGMLRGDFSLRLFNVTPQDEQKFHCLVLSQSLGFQEVLSVEVTLHVAANFSVPVVSAPHSPSQDELTFTCTSINGYPRPNVYWINKTDNSLLDQALQNDTVFLNMRGLYDVVSVLRIARTPSVNIGCCIENVLLQQNLTVGSQTGNDIGERDKITENPVSTGEKNAATWSIL

SEQ ID NO: 344 - protein, 134 aa, synthetic construct
LQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 345 - protein, 201 aa, synthetic construct
LFTVTVPKELYIIEHGSNVTLECNFDTGSHVNLGAITASLQKVENDTSPHRERATLLEEQLPLGKASFHIPQVQVRDEGQYQCIIIYGVAWDYKYLTLKVKASYRKINTHILKVPETDEVELTCQATGYPLAEVSWPNVSVPANTSHSRTPEGLYQVTSVLRLKPPPGRNFSCVFWNTHVRELTLASIDLQSQMEPRTHPT

SEQ ID NO: 346 - protein, 208 aa, synthetic construct [sequence omitted]

SEQ ID NO: 347 - protein, 224 aa, synthetic construct
APLKIQAYFNETADLPCQFANSQNQSLSELVVFWQDQENLVLNEVYLGKEKFDSVHSKYMNRTSFDSDSWTLRLHNLQIKDKGLYQCIIHHKKPTGMIRIHQMNSELSVLANFSQPEIVPISNITENVYINLTCSSIHGYPEPKKMSVLLRTKNSTIEYDGIMQKSQDNVTELYDVSISLSVSFPDVTSNMTIFCILETDKTRLLSSPFSIELEDPQPPPDHIP

SEQ ID NO: 348 - protein, 179 aa, synthetic construct
QLFHLQKELAELRESTSQMHTASSLEKQIGHPSPPPEKKELRKVAHLTGKSNSRSMPLEWEDTYGIVLLSGVKYKKGGLVINETGLYFVYSKVYFRGQSCNNLPLSHKVYMRNSKYPQDLVMMEGKMMSYCTTGQMWARSSYLGAVFNLTSADHLYVNVSELSLVNFEESQTFFGLYKL

SEQ ID NO: 349-355 - skipped (7 entries)

SEQ ID NO: 356 - protein, 100 aa, synthetic construct
  VARIANT 6..100: note = Wherein up to 19 copies of EAAAK may be deleted
EAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 357 - protein, 101 aa, synthetic construct
  VARIANT 7..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAK

SEQ ID NO: 358 - protein, 102 aa, synthetic construct
  VARIANT 7..101: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 359 - protein, 206 aa, synthetic construct
  VARIANT 7..101: note = Wherein up to 19 copies of EAAAK may be deleted
  VARIANT 106..205: note = Wherein up to 19 copies of EAAAK may be deleted
AEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKALEAEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKEAAAKA

SEQ ID NO: 360 - protein, 40 aa, synthetic construct
  VARIANT 3..40: note = Wherein up to 19 copies of KP may be deleted
KPKPKPKPKPKPKPKPKPKPKPKPKPKPKPKPKPKPKPKP

SEQ ID NO: 361 - protein, 40 aa, synthetic construct
  VARIANT 3..40: note = Wherein up to 19 copies of EP may be deleted
EPEPEPEPEPEPEPEPEPEPEPEPEPEPEPEPEPEPEPEP

SEQ ID NO: 362 - protein, 60 aa, synthetic construct
  VARIANT 4..60: note = Wherein up to 19 copies of TPR may be deleted
TPRTPRTPRTPRTPRTPRTPRTPRTPRTPRTPRTPRTPRTPRTPRTPRTPRTPRTPRTPR

SEQ ID NO: 363 - protein, 40 aa, synthetic construct
  VARIANT 3..40: note = Wherein up to 19 copies of AP may be deleted
APAPAPAPAPAPAPAPAPAPAPAPAPAPAPAPAPAPAPAP

SEQ ID NO: 364 - protein, 5 aa, synthetic construct
EAAAK

SEQ ID NO: 365 - protein, 10 aa, synthetic construct
EAAAKEAAAK

SEQ ID NO: 366 - protein, 15 aa, synthetic construct
EAAAKEAAAKEAAAK

SEQ ID NO: 367 - protein, 46 aa, synthetic construct
AEAAAKEAAAKEAAAKEAAAKALEAEAAAKEAAAKEAAAKEAAAKA

SEQ ID NO: 368 - protein, 12 aa, synthetic construct
AEAAAKEAAAKA

SEQ ID NO: 369 - protein, 5 aa, synthetic construct
PAPAP

SEQ ID NO: 370 - protein, 8 aa, synthetic construct
APAPAPAP

SEQ ID NO: 371 - protein, 12 aa, synthetic construct
APAPAPAPAPAP

SEQ ID NO: 372 - protein, 16 aa, synthetic construct
APAPAPAPAPAPAPAP

SEQ ID NO: 373 - protein, 20 aa, synthetic construct
APAPAPAPAPAPAPAPAPAP

SEQ ID NO: 374 - protein, 4 aa, synthetic construct
KPKP

SEQ ID NO: 375 - protein, 8 aa, synthetic construct
KPKPKPKP

SEQ ID NO: 376 - protein, 12 aa, synthetic construct
KPKPKPKPKPKP

SEQ ID NO: 377 - protein, 16 aa, synthetic construct
KPKPKPKPKPKPKPKP

SEQ ID NO: 378 - protein, 20 aa, synthetic construct
KPKPKPKPKPKPKPKPKPKP

SEQ ID NO: 379 - protein, 4 aa, synthetic construct
EPEP

SEQ ID NO: 380 - protein, 8 aa, synthetic construct
EPEPEPEP

SEQ ID NO: 381 - protein, 12 aa, synthetic construct
EPEPEPEPEPEP

SEQ ID NO: 382 - protein, 16 aa, synthetic construct
EPEPEPEPEPEPEPEP

SEQ ID NO: 383 - protein, 20 aa, synthetic construct
EPEPEPEPEPEPEPEPEPEP

SEQ ID NO: 384 - protein, 5 aa, synthetic construct
  VARIANT 4: note = where X is any amino acid other than proline
VPGXG

SEQ ID NO: 385 - protein, 188 aa, synthetic construct [sequence omitted]

SEQ ID NO: 386 - protein, 188 aa, synthetic construct [sequence omitted]

SEQ ID NO: 387 - protein, 189 aa, synthetic construct [sequence omitted]

SEQ ID NO: 388 - protein, 189 aa, synthetic construct [sequence omitted]

SEQ ID NO: 389 - protein, 188 aa, synthetic construct
  VARIANT 5: note = where x is Lys, Phe, or Leu
  VARIANT 7: note = where x is Val or Leu
  VARIANT 8: note = where x is Val or Thr
  VARIANT 9: note = where x is Val or Thr
  VARIANT 12: note = where x is Gly, Asp, Cys, Val, or Ser; and where one or both of x at position 12 and position 13 is not a Cys
  VARIANT 13: note = where x is Gly, Cys, or Asp; and where one or both of x at position 12 and position 13 is not a Cys
MTEYXLXXXGAXXVGKSALTIQLIQNHFVDEYDPTIEDSYRKQVVIDGETCLWDILDTAGQEEYSAMRDQYMRTGEGFLCVFAINNTKSFEDIHHYREQIKRVKDSEDVPMVLVGNKCDLPSRTVDTKQAQDLARSYGIPFIETSAKTRQGVDDAFYTLVREIRKHKEKMSKDGKKKKKKSKTKCVIM

SEQ ID NO: 390 - protein, 9 aa, synthetic construct
VVGADGVGK

SEQ ID NO: 391 - protein, 9 aa, synthetic construct
VVGACGVGK

SEQ ID NO: 392 - protein, 9 aa, synthetic construct
VVGAVGVGK

SEQ ID NO: 393 - protein, 10 aa, synthetic construct
VVVGADGVGK

SEQ ID NO: 394 - protein, 10 aa, synthetic construct
VVVGAVGVGK

SEQ ID NO: 395 - protein, 10 aa, synthetic construct
VVVGACGVGK

SEQ ID NO: 396 - protein, 9 aa, synthetic construct
VTGADGVGK

SEQ ID NO: 397 - protein, 9 aa, synthetic construct
VTGAVGVGK

SEQ ID NO: 398 - protein, 9 aa, synthetic construct
VTGACGVGK

SEQ ID NO: 399 - protein, 10 aa, synthetic construct
VTVGADGVGK

SEQ ID NO: 400 - protein, 10 aa, synthetic construct
VTVGAVGVGK

SEQ ID NO: 401 - protein, 10 aa, synthetic construct
VTVGACGVGK

SEQ ID NO: 402 - protein, 10 aa, synthetic construct
VVVGAGDVGK

SEQ ID NO: 403 - protein, 9 aa, synthetic construct
VVGAGDVGK

SEQ ID NO: 404 - protein, 10 aa, synthetic construct
VVVGARGVGK

SEQ ID NO: 405 - protein, 9 aa, synthetic construct
VVGARGVGK

SEQ ID NO: 406 - protein, 9 aa, synthetic construct
LVVVGADGV

SEQ ID NO: 407 - protein, 9 aa, synthetic construct
LVVVGAVGV

SEQ ID NO: 408 - protein, 9 aa, synthetic construct
LVVVGACGV

SEQ ID NO: 409 - protein, 10 aa, synthetic construct
KLVVVGADGV

SEQ ID NO: 410 - protein, 10 aa, synthetic construct
KLVVVGAVGV

SEQ ID NO: 411 - protein, 10 aa, synthetic construct
KLVVVGACGV

SEQ ID NO: 412 - protein, 9 aa, synthetic construct
LLVVGADGV

SEQ ID NO: 413 - protein, 9 aa, synthetic construct
LLVVGAVGV

SEQ ID NO: 414 - protein, 9 aa, synthetic construct
LLVVGACGV

SEQ ID NO: 415 - protein, 10 aa, synthetic construct
FLVVVGADGV

SEQ ID NO: 416 - protein, 10 aa, synthetic construct
FLVVVGAVGV

SEQ ID NO: 417 - protein, 10 aa, synthetic construct
FLVVVGACGV

SEQ ID NO: 418 - protein, 10 aa, synthetic construct
KLVVVGAGDV

SEQ ID NO: 419 - protein, 10 aa, synthetic construct
KLVVVGARGV

SEQ ID NO: 420 - protein, 10 aa, synthetic construct
GAGDVGKSAL

SEQ ID NO: 421 - protein, 9 aa, synthetic construct
AGDVGKSAL

SEQ ID NO: 422 - protein, 9 aa, synthetic construct
DVGKSALTI

SEQ ID NO: 423 - protein, 10 aa, synthetic construct
GAVGVGKSAL

SEQ ID NO: 424 - protein, 9 aa, synthetic construct
AVGVGKSAL

SEQ ID NO: 425 - protein, 9 aa, synthetic construct
YKLVVVGAV

SEQ ID NO: 426 - protein, 9 aa, synthetic construct
ARGVGKSAL

SEQ ID NO: 427 - protein, 10 aa, synthetic construct
GARGVGKSAL

SEQ ID NO: 428 - protein, 10 aa, synthetic construct
EYKLVVVGAR

SEQ ID NO: 429 - protein, 10 aa, synthetic construct
RGVGKSALTI

SEQ ID NO: 430 - protein, 9 aa, synthetic construct
LVVVGARGV

SEQ ID NO: 431 - protein, 10 aa, synthetic construct
GADGVGKSAL

SEQ ID NO: 432 - protein, 9 aa, synthetic construct
ACGVGKSAL

SEQ ID NO: 433 - protein, 10 aa, synthetic construct
GACGVGKSAL

SEQ ID NO: 434 - protein, 10 aa, synthetic construct
VVVGAGGVGK

SEQ ID NO: 435 - protein, 10 aa, synthetic construct
VVVGACGVGK

SEQ ID NO: 436 - protein, 10 aa, synthetic construct
VVVGADGVGK

SEQ ID NO: 437 - protein, 10 aa, synthetic construct
VVVGARGVGK

SEQ ID NO: 438 - protein, 10 aa, synthetic construct
VVVGAVGVGK

SEQ ID NO: 439 - protein, 9 aa, synthetic construct
VVGAVGVGK

SEQ ID NO: 440 - protein, 10 aa, synthetic construct
VVVGARGVGK

SEQ ID NO: 441 - protein, 9 aa, synthetic construct
VVGARGVGK

SEQ ID NO: 442 - protein, 10 aa, synthetic construct
VVVGAVGVGK

SEQ ID NO: 443 - protein, 9 aa, synthetic construct
VVGAVGVGK

SEQ ID NO: 444 - protein, 10 aa, synthetic construct
VVVGACGVGK

SEQ ID NO: 445 - protein, 10 aa, synthetic construct
VVVGADGVGK

SEQ ID NO: 446 - protein, 10 aa, synthetic construct
VVVGARGVGK

SEQ ID NO: 447 - protein, 10 aa, synthetic construct
VVVGAVGVGK

SEQ ID NO: 448 - protein, 9 aa, synthetic construct
VVGAVGVGK

SEQ ID NO: 449 - protein, 10 aa, synthetic construct
GADGVGKSAL

SEQ ID NO: 450 - protein, 10 aa, synthetic construct
GARGVGKSAL

SEQ ID NO: 451 - protein, 9 aa, synthetic construct
AVGVGKSAL

SEQ ID NO: 452 - protein, 10 aa, synthetic construct
GAVGVGKSAL

SEQ ID NO: 453 - protein, 9 aa, synthetic construct
GAVGVGKSA

SEQ ID NO: 454 - protein, 9 aa, synthetic construct
GACGVGKSA

SEQ ID NO: 455 - protein, 10 aa, synthetic construct
GADGVGKSAL

SEQ ID NO: 456 - protein, 9 aa, synthetic construct
GADGVGKSA

SEQ ID NO: 457 - protein, 9 aa, synthetic construct
AVGVGKSAL

SEQ ID NO: 458 - protein, 10 aa, synthetic construct
GAVGVGKSAL

SEQ ID NO: 459 - protein, 9 aa, synthetic construct
GAVGVGKSA

SEQ ID NO: 460 - protein, 9 aa, synthetic construct
GACGVGKSA

SEQ ID NO: 461 - protein, 10 aa, synthetic construct
GADGVGKSAL

SEQ ID NO: 462 - protein, 10 aa, synthetic construct
GAVGVGKSAL

SEQ ID NO: 463 - protein, 9 aa, synthetic construct
GAVGVGKSA

SEQ ID NO: 464 - protein, 10 aa, synthetic construct
GADGVGKSAL

SEQ ID NO: 465 - protein, 9 aa, synthetic construct
GADGVGKSA

SEQ ID NO: 466 - protein, 9 aa, synthetic construct
VVGAVGVGK

SEQ ID NO: 467 - protein, 10 aa, synthetic construct
VVVGAVGVGK

SEQ ID NO: 468 - protein, 9 aa, synthetic construct
VGAVGVGKS

SEQ ID NO: 469 - protein, 10 aa, synthetic construct
VGAVGVGKSA

SEQ ID NO: 470 - protein, 9 aa, synthetic construct
AVGVGKSAL

SEQ ID NO: 471 - protein, 10 aa, synthetic construct
AVGVGKSALT

SEQ ID NO: 472 - protein, 10 aa, synthetic construct
GAVGVGKSAL

SEQ ID NO: 473 - protein, 9 aa, synthetic construct
GAVGVGKSA

SEQ ID NO: 474 - protein, 10 aa, synthetic construct
LVVVGAVGVG

SEQ ID NO: 475 - protein, 9 aa, synthetic construct
LVVVGAVGV

SEQ ID NO: 476 - protein, 10 aa, synthetic construct
KLVVVGAVGV

SEQ ID NO: 477 - protein, 9 aa, synthetic construct
KLVVVGAVG

SEQ ID NO: 478 - protein, 9 aa, synthetic construct
VVGADGVGK

SEQ ID NO: 479 - protein, 9 aa, synthetic construct
VVGACGVGK

SEQ ID NO: 480 - protein, 9 aa, synthetic construct
VVGAVGVGK

SEQ ID NO: 481 - protein, 10 aa, synthetic construct
VVVGADGVGK

SEQ ID NO: 482 - protein, 10 aa, synthetic construct
VVVGAVGVGK

SEQ ID NO: 483 - protein, 10 aa, synthetic construct
VVVGACGVGK

SEQ ID NO: 484 - protein, 9 aa, synthetic construct
VTGADGVGK

SEQ ID NO: 485 - protein, 9 aa, synthetic construct
VTGAVGVGK

SEQ ID NO: 486 - protein, 9 aa, synthetic construct
VTGACGVGK

SEQ ID NO: 487 - protein, 10 aa, synthetic construct
VTVGADGVGK

SEQ ID NO: 488 - protein, 10 aa, synthetic construct
VTVGAVGVGK

SEQ ID NO: 489 - protein, 10 aa, synthetic construct
VTVGACGVGK

SEQ ID NO: 490 - protein, 10 aa, synthetic construct
VVVGAGDVGK

SEQ ID NO: 491 - protein, 9 aa, synthetic construct
VVGAGDVGK

SEQ ID NO: 492 - protein, 10 aa, synthetic construct
VVVGARGVGK

SEQ ID NO: 493 - protein, 9 aa, synthetic construct
VVGARGVGK

SEQ ID NO: 494 - protein, 9 aa, synthetic construct
LVVVGADGV

SEQ ID NO: 495 - protein, 9 aa, synthetic construct
LVVVGAVGV

SEQ ID NO: 496 - protein, 9 aa, synthetic construct
LVVVGACGV

SEQ ID NO: 497 - protein, 10 aa, synthetic construct
KLVVVGADGV

SEQ ID NO: 498 - protein, 10 aa, synthetic construct
KLVVVGAVGV

SEQ ID NO: 499 - protein, 10 aa, synthetic construct
KLVVVGACGV

SEQ ID NO: 500 - protein, 9 aa, synthetic construct
LLVVGADGV

SEQ ID NO: 501 - protein, 9 aa, synthetic construct
LLVVGAVGV

SEQ ID NO: 502 - protein, 9 aa, synthetic construct
LLVVGACGV

SEQ ID NO: 503 - protein, 10 aa, synthetic construct
FLVVVGADGV

SEQ ID NO: 504 - protein, 10 aa, synthetic construct
FLVVVGAVGV

SEQ ID NO: 505 - protein, 10 aa, synthetic construct
FLVVVGACGV

SEQ ID NO: 506 - protein, 10 aa, synthetic construct
GAGDVGKSAL

SEQ ID NO: 507 - protein, 9 aa, synthetic construct
AGDVGKSAL

SEQ ID NO: 508 - protein, 9 aa, synthetic construct
DVGKSALTI

SEQ ID NO: 509 - protein, 10 aa, synthetic construct
GAVGVGKSAL

SEQ ID NO: 510 - protein, 9 aa, synthetic construct
AVGVGKSAL

SEQ ID NO: 511 - protein, 9 aa, synthetic construct
YKLVVVGAV

SEQ ID NO: 512 - protein, 9 aa, synthetic construct
ARGVGKSAL

SEQ ID NO: 513 - protein, 10 aa, synthetic construct
GARGVGKSAL

SEQ ID NO: 514 - protein, 10 aa, synthetic construct
EYKLVVVGAR

SEQ ID NO: 515 - protein, 10 aa, synthetic construct
RGVGKSALTI

SEQ ID NO: 516 - protein, 9 aa, synthetic construct
LVVVGARGV

SEQ ID NO: 517 - protein, 10 aa, synthetic construct
GADGVGKSAL

SEQ ID NO: 518 - protein, 9 aa, synthetic construct
ACGVGKSAL

SEQ ID NO: 519 - protein, 10 aa, synthetic construct
GACGVGKSAL

(19) United States
(12) Patent Application Publication
(10) Pub. No.: US 2026/0109958 A1
(43) Pub. Date: Apr. 23, 2026

(54) T-Cell Modulatory Polypeptides and Methods of Use Thereof

(71) Applicant: Cue Biopharma, Inc., Boston, MA (US)

(72) Inventors: Chee Meng Low, Boston, MA (US); Ahmet Selim Vakkasoglu, Boston, MA (US)

(21) Appl. No.: 19/422,115

(22) Filed: Dec. 16, 2025

Related U.S. Application Data
(63) Continuation of application No. PCT/US2024/035144, filed on Jun. 21, 2024.
(60) Provisional application No. 63/522,877, filed on Jun. 23, 2023.

(51) Int. Cl. C12N 9/14; A61K 38/00; C07K 14/55; C07K 14/705; C07K 14/74; C07K 16/28

(52) U.S. Cl. CPC C12N 9/14; C07K 14/55; C07K 14/70503; C07K 14/70539; C07K 16/2818; C07K 16/2827; A61K 38/00; C07K 2317/569; C07K 2317/622; C07K 2319/30; C12Y 306/05002

(57) ABSTRACT

The present disclosure provides a peptide-major histocompatibility complex (pMHC) polypeptide comprising a KRAS peptide and class I MHC polypeptides. The present disclosure provides fusion molecules comprising a pMHC polypeptide and a heterologous fusion partner. The present disclosure provides single-chain T-cell modulatory polypeptides that comprise a pMHC polypeptide, one or more immunomodulatory polypeptide, and an immunoglobulin (Ig) Fc or a non-Ig scaffold. A TMP is useful for modulating the activity of a T cell, for modulating an immune response in an individual, and/or for treating an individual who has a KRAS-associated cancer.

### CROSS-REFERENCE

This application is a continuation of PCT Application No. PCT/US2024/035144, filed Jun. 21 2024, which claims the benefit of U.S. Provisional Patent Application No. 63/522,877, filed Jun. 23, 2023, which applications are incorporated herein by reference in their entirety.

### INCORPORATION-BY-REFERENCE OF MATERIAL ELECTRONICALLY SUBMITTED

A Sequence Listing is provided herewith as a Sequence Listing XML, “CUEB-154WO_SEQLIST” created on Jun. 21, 2024 and having a size of 409,458 bytes. The contents of the Sequence Listing XML are incorporated by reference herein in their entirety.

### INTRODUCTION

An adaptive immune response involves the engagement of the T cell receptor (TCR), present on the surface of a T cell, with a small peptide antigen non-covalently presented on the surface of an antigen presenting cell (APC) by a major histocompatibility complex (MHC; also referred to in humans as a human leukocyte antigen (HLA) complex). This engagement represents the immune system's targeting mechanism and is a requisite molecular interaction for T cell modulation (activation or inhibition) and effector function. Following epitope-specific cell targeting, the targeted T cells are activated through engagement of costimulatory proteins found on the APC with counterpart costimulatory proteins the T cells. Both signals—epitope/TCR binding and engagement of APC costimulatory proteins with T cell costimulatory proteins—are required to drive T cell specificity and activation or inhibition. The costimulatory proteins on the APC also are referred to as “immunomodulatory” proteins because they modulate the activity of the T cell when they bind the costimulatory protein on the T cell, with the specific modulation being a function of which immunomodulatory protein on the APC binds to which costimulatory protein on the T cell. The TCR is specific for a given epitope; however, the T cell's costimulatory proteins are not epitope-specific and instead is generally expressed on all T cells or on large T cell subsets.

### SUMMARY

The present disclosure provides a single-chain T-cell modulatory polypeptide (TMP) that comprises: (i) a peptide-major histocompatibility complex (pMHC) polypeptide comprising a KRAS peptide, (ii) one or more immunomodulatory polypeptides, and (iii) an immunoglobulin (Ig) Fc or a non-Ig scaffold. A TMP is useful for modulating the activity of a T cell, and for modulating an immune response in an individual.

### BRIEF DESCRIPTION OF THE DRAWINGS

FIGS. 1A-1J provide schematic depictions of disulfide-linked TMPs.

FIGS. 2A-2B provide an amino acid sequence of a wild-type human β2M polypeptide (FIG. 2A; SEQ ID NO:42) and an amino acid sequence of a β2M polypeptide with an R12C substitution (FIG. 2B; SEQ ID NO:43.

FIGS. 3A-3E provide amino acid sequences of wild-type HLA-A*0201 (FIG. 3A; SEQ ID NO:44) and variants (FIGS. 3B-3E; SEQ ID NOs:45-48, respectively).

FIGS. 4A-4E provide amino acid sequences of wild-type HLA-A*1101 (FIG. 4A; SEQ ID NO:49) and variants (FIGS. 4B-4E; SEQ ID NOs:50-53, respectively).

FIGS. 5A-5E provide amino acid sequences of wild-type HLA-A*2402 (FIG. 5A; SEQ ID NO:54) and variants (FIGS. 5B-5E; SEQ ID NOs:55-58, respectively).

FIGS. 6A-6E provide amino acid sequences of wild-type HLA-A*3303 (FIG. 6A; SEQ ID NO:59) and variants (FIGS. 6B-6E; SEQ ID NOs:60-63, respectively).

FIGS. 7A-7E provide amino acid sequences of wild-type HLA-A*0301 (FIG. 7A; SEQ ID NO:64) and variants (FIGS. 7B-7E; SEQ ID NOs:65-68, respectively).

FIGS. 8A-8D provide an alignment of amino acid sequences of wild-type (FIGS. 8A and continued in 8B; SEQ ID NOs:69-77, respectively) and variant (FIGS. 8C and continued in 8D; SEQ ID NO:78-86, respectively) HLA-A polypeptides.

FIGS. 9A-9D provide an alignment of amino acid sequences of wild-type (FIGS. 9A and continued in 9B; SEQ ID NOs:87-93, respectively) and variant (FIGS. 9C and continued in 9D; SEQ ID NOs:94-100, respectively) HLA-B polypeptides.

FIGS. 10A-10D provide an alignment of amino acid sequences of wild-type (FIGS. 10A and continued in 10B; SEQ ID NOs:101-109, respectively) and variant (FIGS. 10C and continued in 10D; SEQ ID NOs:110-118, respectively) HLA-C polypeptides.

FIGS. 11A-11E provide amino acid sequences of wild-type (FIG. 11A; SEQ ID NO: 119) HLA-E heavy chains and variants (FIGS. 11B-11E; SEQ ID NOs:120-123, respectively).

FIGS. 12A-12E provide amino acid sequences of wild-type (FIG. 12A; SEQ ID NO: 124) HLA-E heavy chains and variants (FIGS. 12B-12E; SEQ ID NOs:125-128, respectively).

FIGS. 13A-13D provide amino acid sequences of wild-type (FIG. 13A; SEQ ID NO: 129 and FIG. 13C; SEQ ID NO:131) and variant (FIG. 13B; SEQ ID NO: 130 and FIG. 13D; SEQ ID NO:132) HLA-G heavy chains.

FIG. 14 provides schematic depictions of examples of positions of immunomodulatory polypeptides in TMPs.

FIGS. 15A-15L provide amino acid sequences of immunoglobulin Fc polypeptides (SEQ ID NOs:133-144, respectively).

FIGS. 16A-16D provide amino acid sequences of IL-2Rα (FIG. 16A; SEQ ID NO: 145), IL-2Rβ (FIG. 16B; SEQ ID NO: 146), and IL-2Rγ (FIG. 16C; SEQ ID NO: 147), and an amino acid sequence of wild-type IL-2 (FIG. 16D; SEQ ID NO: 148).

FIGS. 17A-17E provide amino acid sequences of some examples of single-chain TMPs of the present disclosure (SEQ ID NOs:149-153) (“G2C”, “(G4S)3”, “AAAGG”, “(AP)4”, “(G4S)4”, “GCGGS(GGGS)2”, “(GGGGS)3”, and “(GGGGS)4” linkers are SEQ ID NOs:154-163, respectively) (“KRAS epitope” is SEQ ID NO:162; “KRAS(71-6; G12D)” is SEQ ID NO: 163).

FIGS. 18A-18F provide amino acid sequences of various constructs used in the Examples (SEQ ID NOs:164-169) (“(G4S)3”, “AAAGG”, “(AP)4”, “(G4S)4”, and “GCGGS(GGGS)2” linkers are SEQ ID NOs:155-159, respectively) (“KRAS (G12D)” and “KRAS (G12V)” are SEQ ID NOs: 163 and 170, respectively).

FIG. 19 depicts temperature stability of various constructs described in the Examples.

FIG. 20 depicts expansion of peptide-specific T cells induced by various constructs described in the Examples.

FIGS. 21A-21B depict expansion of peptide-specific T cells induced by various constructs described in Example 3.

### DEFINITIONS

The terms “polynucleotide” and “nucleic acid,” used interchangeably herein, refer to a polymeric form of nucleotides of any length, either ribonucleotides or deoxyribonucleotides. Thus, this term includes, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases.

The terms “peptide,” “polypeptide,” and “protein” are used interchangeably herein, and refer to a polymeric form of amino acids of any length, which can include coded and non-coded amino acids, chemically or biochemically modified or derivatized amino acids, and polypeptides having modified peptide backbones. Furthermore, as used herein, a “polypeptide” refers to a protein that includes modifications, such as deletions, additions, and substitutions (generally conservative in nature as would be known to a person in the art) to the native sequence, as long as the protein maintains the desired activity. These modifications can be deliberate, as through site-directed mutagenesis, or can be accidental, such as through mutations of hosts that produce the proteins, or errors due to polymerase chain reaction (PCR) amplification or other recombinant DNA methods. References herein to a specific residue or residue number in a known polypeptide are understood to refer to the amino acid at that position in the wild-type polypeptide. To the extent that the sequence of the wild-type polypeptide is altered, either by addition or deletion of one or more amino acids, one of ordinary skill will understand that a reference to the specific residue or residue number will be correspondingly altered so as to refer to the same specific amino acid in the altered polypeptide, which would be understood to reside at an altered position number. For example, if an MHC class I polypeptide is altered by the addition of one amino acid at the N-terminus, then a reference to position 84 or a specific residue at position 84, will be understood to indicate the amino acids that are at position 85 on the altered polypeptide. Likewise, a reference herein to substitution of a specific amino acid at a specific position, e.g., Y84, is understood to refer to a substitution of an amino acid for the amino acid at position 84 in the wild-type polypeptide. A Y84A substitution is thus understood to be a substitution of an Ala residue for the Tyr residue that is present in the wild-type sequence. If, e.g., the wild-type polypeptide is altered to change the amino acid at position 84 from its wild-type amino acid to an alternate amino acid, then the substitution for the amino acid at position 84 will be understood to refer to the substitution for the alternate amino acid. If in such case the polypeptide is also altered by the addition or deletion of one or more amino acids, then the reference to the substitution will be understood to refer to the substitution for the alternate amino acid at the altered position number. A reference to a “non-naturally occurring Cys residue” in a polypeptide, e.g., an MHC class I polypeptide, means that the polypeptide comprises a Cys residue in a location where there is no Cys in the corresponding wild-type polypeptide. This can be accomplished through routine protein engineering in which a cysteine is substituted for the amino acid that occurs in the wild-type sequence.

A polynucleotide or polypeptide has a certain percent “sequence identity” to another polynucleotide or polypeptide, meaning that, when aligned, that percentage of bases or amino acids are the same, and in the same relative position, when comparing the two sequences. Sequence identity can be determined in a number of different ways. To determine sequence identity, sequences can be aligned using various convenient methods and computer programs (e.g., BLAST, T-COFFEE, MUSCLE, MAFFT, etc.), available over the world wide web at sites including ncbi.nlm.nili.gov/BLAST, ebi.ac.uk/Tools/msa/tcoffee/, ebi.ac.uk/Tools/msa/muscle/, mafft.cbrc.jp/alignment/software/. See, e.g., Altschul et al. (1990), J. Mol. Biol. 215:403-10. Unless otherwise stated, “sequence identity” as referred to herein is determined by BLAST (Basic Local Alignment Search Tool), as described in Altschul et al. ((1990) J. Mol. Biol. 215:403), using default parameters.

The term “conservative amino acid substitution” refers to the interchangeability in proteins of amino acid residues having similar side chains. For example, a group of amino acids having aliphatic side chains consists of glycine, alanine, valine, leucine, and isoleucine; a group of amino acids having aliphatic-hydroxyl side chains consists of serine and threonine; a group of amino acids having amide containing side chains consisting of asparagine and glutamine; a group of amino acids having aromatic side chains consists of phenylalanine, tyrosine, and tryptophan; a group of amino acids having basic side chains consists of lysine, arginine, and histidine; a group of amino acids having acidic side chains consists of glutamate and aspartate; and a group of amino acids having sulfur containing side chains consists of cysteine and methionine. Exemplary conservative amino acid substitution groups are: valine-leucine-isoleucine, phenylalanine-tyrosine, lysine-arginine, alanine-valine-glycine, and asparagine-glutamine.

“T cell” includes all types of immune cells expressing CD3, including T-helper cells (CD4⁺ cells), cytotoxic T-cells (CD8⁺ cells), T-regulatory cells (Treg), and NK-T cells.

The term “immunomodulatory polypeptide” (also referred to herein as a “MOD”), as used herein, means a polypeptide that specifically binds a cognate costimulatory polypeptide on a T cell, thereby providing a signal which, in addition to the primary signal provided by, for instance, binding of a TCR/CD3 complex with a major histocompatibility complex (MHC) polypeptide loaded with peptide, mediates a T cell response, including, but not limited to, proliferation, activation, differentiation, and the like. As discussed herein, a MOD can include, but is not limited to wild-type or variants of wild-type polypeptides such as a cytokine (e.g., IL-2), CD7, B7-1 (CD80), B7-2 (CD86), PD-L1, PD-L2, 4-1BBL, OX40L, Fas ligand (FasL), inducible costimulatory ligand (ICOS-L), intercellular adhesion molecule (ICAM), CD30L, CD40, CD70, CD83, HLA-G, MICA, MICB, HVEM, lymphotoxin beta receptor, 3/TR6, ILT3, ILT4, HVEM, an agonist or antibody that binds Toll ligand receptor, and a ligand that specifically binds with B7-H3. A MOD of a TMP can bind a cognate costimulatory polypeptide (i.e., a “co-MOD”) that is present on a target T cell.

As used herein the term “in vivo” refers to any process or procedure occurring inside of the body.

As used herein, “in vitro” refers to any process or procedure occurring outside of the body.

“Heterologous,” as used herein, means a nucleotide or polypeptide that is not found in the native nucleic acid or protein, respectively.

“Recombinant,” as used herein, means that a particular nucleic acid (DNA or RNA) is the product of various combinations of cloning, restriction, polymerase chain reaction (PCR) and/or ligation steps resulting in a construct having a structural coding or non-coding sequence distinguishable from endogenous nucleic acids found in natural systems. DNA sequences encoding polypeptides can be assembled from cDNA fragments or from a series of synthetic oligonucleotides, to provide a synthetic nucleic acid which is capable of being expressed from a recombinant transcriptional unit contained in a cell or in a cell-free transcription and translation system.

The terms “recombinant expression vector,” or “DNA construct” are used interchangeably herein to refer to a DNA molecule comprising a vector and at least one insert. Recombinant expression vectors are usually generated for the purpose of expressing and/or propagating the insert(s), or for the construction of other recombinant nucleotide sequences. The insert(s) may or may not be operably linked to a promoter sequence and may or may not be operably linked to DNA regulatory sequences.

As used herein, the term “affinity” refers to the equilibrium constant for the reversible binding of two agents (e.g., an antibody and an antigen) and is expressed as a dissociation constant (K_{D}). As used herein, the term “avidity” refers to the resistance of a complex of two or more agents to dissociation after dilution. The terms “immunoreactive” and “preferentially binds” are used interchangeably herein with respect to antibodies and/or antigen-binding fragments.

The term “binding,” as used herein (e.g., with reference to binding of a TMP to a polypeptide (e.g., a T-cell receptor) on a T cell; or with reference to binding of a pMHC polypeptide to a TCR), refers to a non-covalent interaction between two molecules. Non-covalent binding refers to a direct association between two molecules, due to, for example, electrostatic, hydrophobic, ionic, and/or hydrogen-bond interactions, including interactions such as salt bridges and water bridges. “Affinity” refers to the strength of non-covalent binding, increased binding affinity being correlated with a lower K_{D}. “Specific binding” generally refers to binding of a ligand to a moiety that is than its designated binding site or receptor. “Non-specific binding” generally refers to binding of a ligand to a moiety other than its designated binding site or receptor. “Covalent binding” or “covalent bond,” as used herein, refers to the formation of one or more covalent chemical binds between two different molecules.

The terms “treatment”, “treating” and the like are used herein to generally mean obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment” as used herein covers any treatment of a disease or symptom in a mammal, and includes: (a) preventing the disease or symptom from occurring in a subject which may or may not be predisposed to acquiring the disease or symptom but has not yet been diagnosed as having it; (b) inhibiting the disease or one or more symptoms associated with the disease, e.g., arresting its development; and/or (c) relieving the disease, i.e., causing regression of the disease. The therapeutic agent may be administered before, during and/or after the onset of disease or injury. The treatment of ongoing disease, where the treatment stabilizes or reduces the undesirable clinical symptoms of the patient, is of particular interest. Such treatment is desirably performed prior to complete loss of function in the affected tissues. The subject therapy will desirably be administered during the symptomatic stage of the disease, and in some cases after the symptomatic stage of the disease.

The terms “individual,” “subject,” “host,” and “patient,” are used interchangeably herein and refer to any mammalian subject for whom diagnosis, treatment, or therapy is desired. Mammals include, e.g., humans, non-human primates, rodents (e.g., rats; mice), lagomorphs (e.g., rabbits), ungulates (e.g., cows, sheep, pigs, horses, goats, and the like), etc. Unless otherwise indicated, the terms “individual,” “subject,” “host,” and “patient,” refer to a human.

Unless indicated otherwise, the term “substantially” is intended to encompass both “wholly” and “largely but not wholly”. For example, an Ig Fc that “substantially does not induce cell lysis” means an Ig Fc that induces no cell lysis at all or that largely does not induce cell lysis.

As used herein, the term “about” used in connection with an amount indicates that the amount can vary by 10% of the stated amount. For example, “about 100” means an amount of from 90-110. Where about is used in the context of a range, the “about” used in reference to the lower amount of the range means that the lower amount includes an amount that is 10% lower than the lower amount of the range, and “about” used in reference to the higher amount of the range means that the higher amount includes an amount 10% higher than the higher amount of the range. For example, from about 100 to about 1000 means that the range extends from 90 to 1100.

As used herein, the term “MHC heavy chain polypeptide” means collectively the domains of an MHC heavy chain polypeptide that are present in a pMHC polypeptide. For example, an MHC heavy chain polypeptide can comprise α1, α2 and α3 domains.

Before the present disclosure is further described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limit of that range and any other stated or intervening value in that stated range, is encompassed within the disclosure. The upper and lower limits of these smaller ranges may independently be included in the smaller ranges, and are also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can also be used in the practice or testing of the present disclosure, the preferred methods and materials are now described. All publications mentioned herein are incorporated herein by reference to disclose and describe the methods and/or materials in connection with which the publications are cited.

As used herein and in the appended claims, the singular forms “a,” “an,” and “the” include plural referents unless the context clearly dictates otherwise. Thus, for example, reference to a “pMHC polypeptide” includes a plurality of such polypeptides and reference to “the immunomodulatory polypeptide” includes reference to one or more immunomodulatory polypeptides and equivalents thereof known to those skilled in the art, and so forth. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation.

The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. For example, if the range 10-15 is disclosed, then 11, 12, 13, and 14 are also disclosed. All methods described herein can be performed in any suitable order unless otherwise indicated herein or otherwise clearly contradicted by context. The use of any and all examples, or exemplary language (e.g., “such as”) provided herein, is intended merely to better illuminate the embodiments of the disclosure and does not pose a limitation on the scope of the disclosure unless otherwise claimed.

The term “and/or” as used herein a phrase such as “A and/or B” is intended to include both A and B; A or B; A (alone); and B (alone). Likewise, the term “and/or” as used herein a phrase such as “A, B, and/or C” is intended to encompass each of the following embodiments: A, B, and C; A, B, or C; A or C; A or B; B or C; A and C; A and B; B and C; A (alone); B (alone); and C (alone).

It is understood that aspects and embodiments of the present disclosure described herein include “comprising,” “consisting,” and “consisting essentially of” aspects and embodiments.

It is appreciated that certain features of the disclosure, which are, for clarity, described in the context of separate embodiments, may also be provided in combination in a single embodiment. Conversely, various features of the disclosure, which are, for brevity, described in the context of a single embodiment, may also be provided separately or in any suitable sub-combination. All combinations of the embodiments pertaining to the disclosure are specifically embraced by the present disclosure and are disclosed herein just as if each and every combination was individually and explicitly disclosed. In addition, all sub-combinations of the various embodiments and elements thereof are also specifically embraced by the present disclosure and are disclosed herein just as if each and every such sub-combination was individually and explicitly disclosed herein.

The publications discussed herein are provided solely for their disclosure prior to the filing date of the present application. Nothing herein is to be construed as an admission that the present disclosure is not entitled to antedate such publication. Further, the dates of publication provided may be different from the actual publication dates which may need to be independently confirmed.

### DETAILED DESCRIPTION

The present disclosure provides a single-chain T-cell modulatory polypeptide (TMP) that comprises: (i) a peptide-major histocompatibility complex (pMHC) polypeptide comprising a KRAS peptide, (ii) one or more immunomodulatory polypeptides, and (iii) an immunoglobulin (Ig) Fc or a non-Ig scaffold. A TMP is useful for modulating the activity of a T cell, and for modulating an immune response in an individual.

### T-Cell Modulatory Polypeptides

The present disclosure provides a single-chain T-cell modulatory polypeptide (TMP) comprising: a) a pMHC polypeptide comprising: i) a KRAS peptide; ii) beta-2 microglobulin (β2M) polypeptide; and iii) an MHC class I heavy chain polypeptide; and b) one or more immunomodulatory polypeptides (MODs). The present disclosure provides a single-chain TMP comprising: a) a pMHC polypeptide comprising a KRAS peptide, a β2M polypeptide, and an MHC class I heavy chain polypeptide; b) one or more MODs; and c) an Ig Fc polypeptide or a non-immunoglobulin scaffold component. The TMP can comprise one or more independently selected peptide linkers between any two of the aforementioned components.

Generally speaking, a TMP binds to a T cell having a co-immunomodulatory polypeptide (co-MOD) and a TCR that binds the peptide/MHC complex of the TMP with an affinity that is greater (e.g., 25% greater) than the affinity with which the same TMP binds a second T cell that has the same co-MOD but has a TCR that substantially does not bind the peptide/MHC complex.

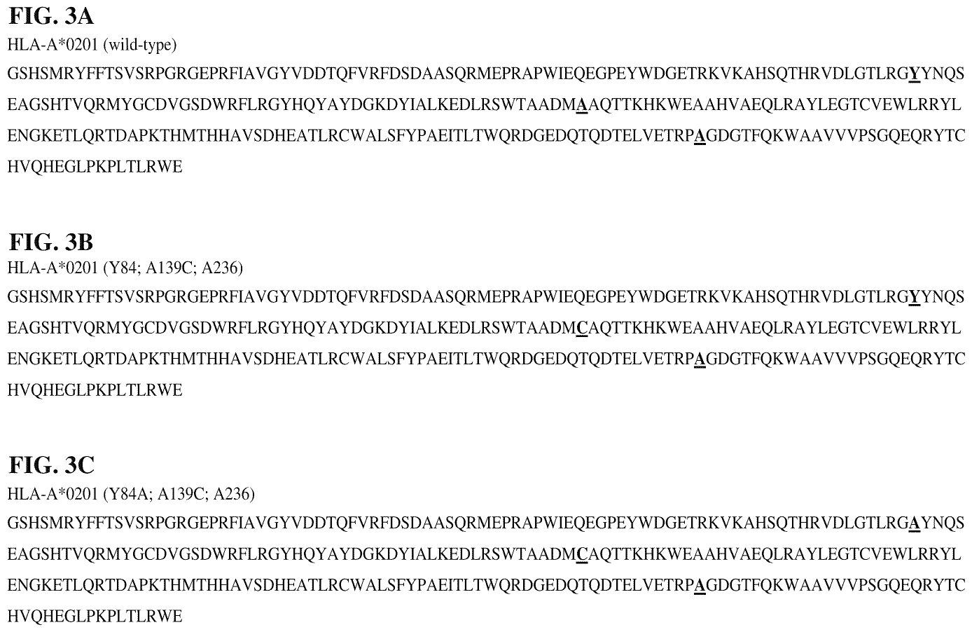

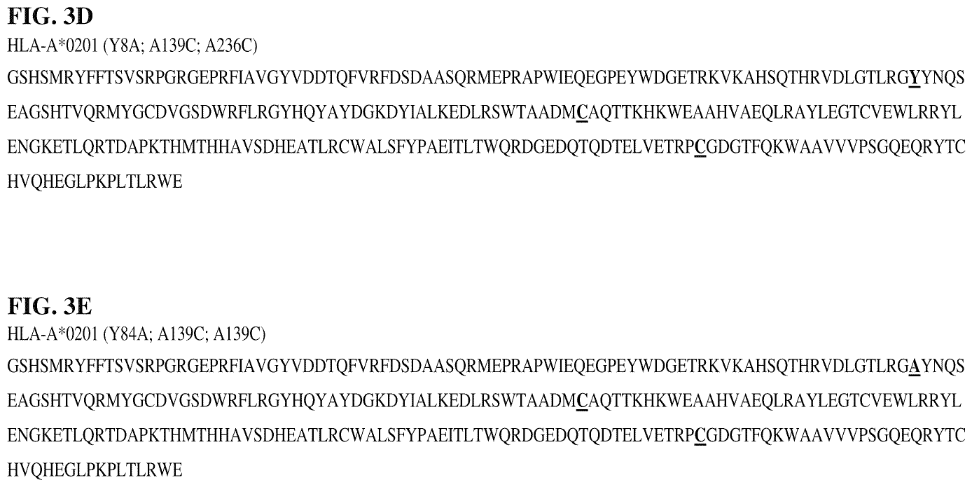

In some cases, a TMP comprises, in order from N-terminus to C-terminus, the following components: a) pMHC polypeptide, where the pMHC polypeptide comprises, in order from N-terminus to C-terminus: i) a KRAS peptide; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; and b) one or more MODs.

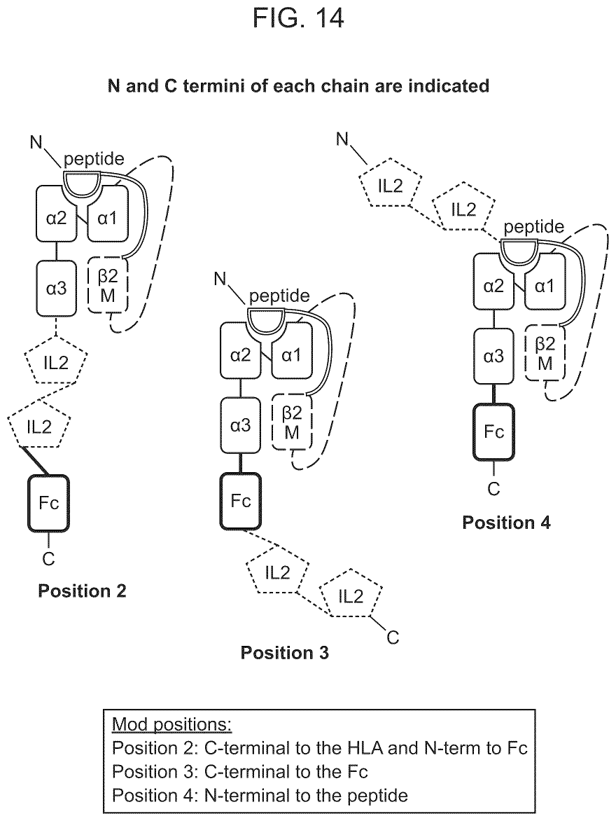

In some cases, a TMP comprises, in order from N-terminus to C-terminus, the following components: a) pMHC polypeptide, where the pMHC polypeptide comprises, in order from N-terminus to C-terminus: i) a KRAS peptide; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) one or more MODs; and c) an Ig Fc polypeptide. This arrangement of components is referred to as MOD Position 2 in FIG. 14. The TMP can include one or more additional peptide linkers. For example, the TMP can include: i) a peptide linker between the MHC class I heavy chain polypeptide and the MOD; and/or ii) a peptide linker between the MOD and the Ig Fc polypeptide. Where the TMP comprises two MODs in tandem, the TMP can include a peptide linker between the two MODs.

In some cases, a TMP comprises, in order from N-terminus to C-terminus, the following components: a) pMHC polypeptide, where the pMHC polypeptide comprises, in order from N-terminus to C-terminus: i) a KRAS peptide; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) an Ig Fc polypeptide; and c) one or more MODs. This arrangement of components is referred to as MOD Position 3 in FIG. 14. The TMP can include one or more additional peptide linkers. For example, the TMP can include: i) a peptide linker between the MHC class I heavy chain polypeptide and the Ig Fc polypeptide; and/or ii) a peptide linker between the Ig Fc polypeptide and the MOD. Where the TMP comprises two MODs in tandem, the TMP can include a peptide linker between the two MODs.

A MOD may comprise either a wild type (“wt”) MOD or a variant of a wt MOD. Where a MOD comprises a variant, it may exhibit reduced binding to its co-MOD, including e.g., reduced binding to one or more chains or domains of the co-MOD. In such cases, combination of the reduced affinity of the MOD for its co-MOD, and the affinity of the peptide for a TCR, may provide for enhanced selectivity of a TMP. Binding affinity between a MOD and its co-MOD can be determined by bio-layer interferometry (BLI) using purified MOD and purified co-MOD. Binding affinity between a MOD present in a TMP and its co-MOD can be determined by BLI using purified TMP and the co-MOD. BLI methods are well known to those skilled in the art. See, e.g., Lad et al. (2015) J. Biomol. Screen. 20(4):498-507; and Shah and Duncan (2014) J. Vis. Exp. 18:e51383. Unless otherwise stated herein, the affinity of a MOD for a co-MOD, or the affinity of a MOD on a TMP for a co-MOD, is determined using BLI as described in in published PCT application WO 2020/132138, published Jun. 25, 2020. See, e.g., paragraphs [0056]-[0057].

pMHC Polypeptides

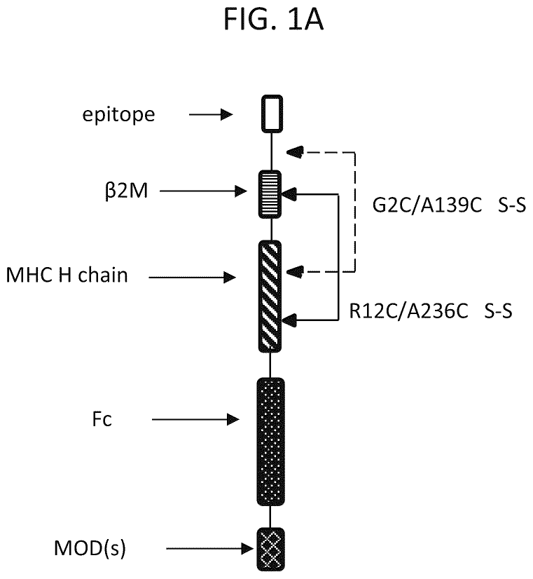

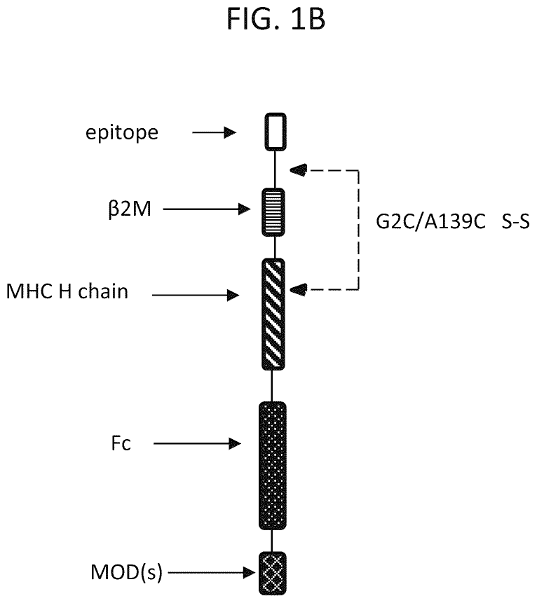

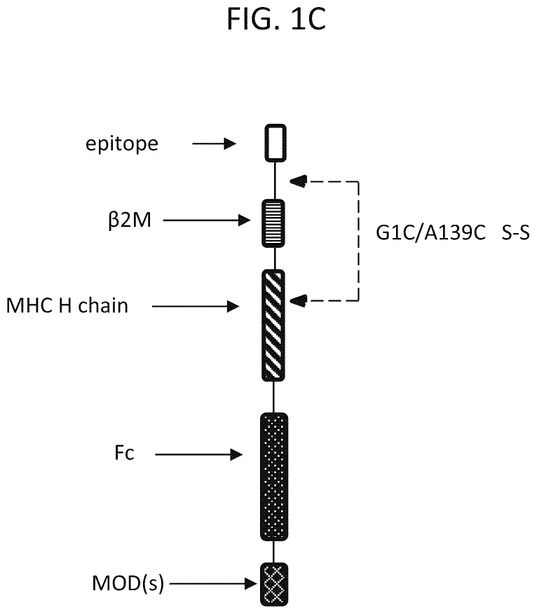

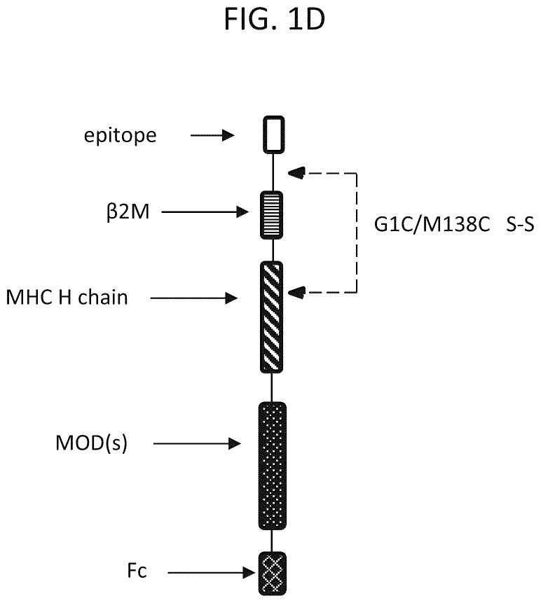

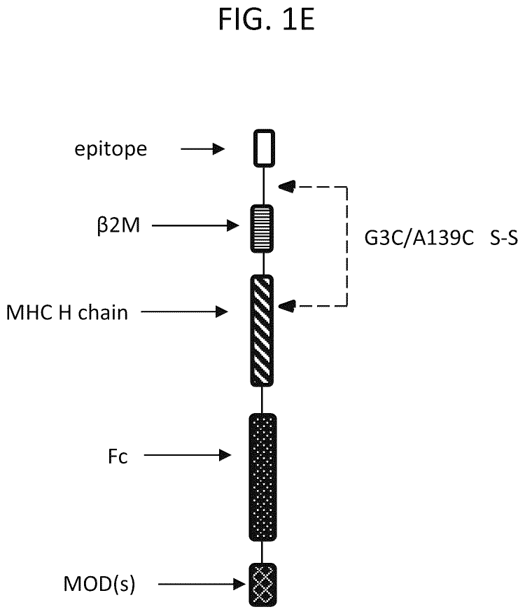

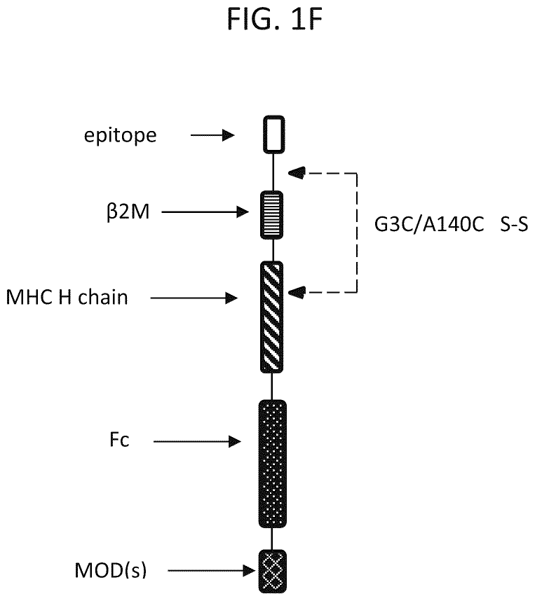

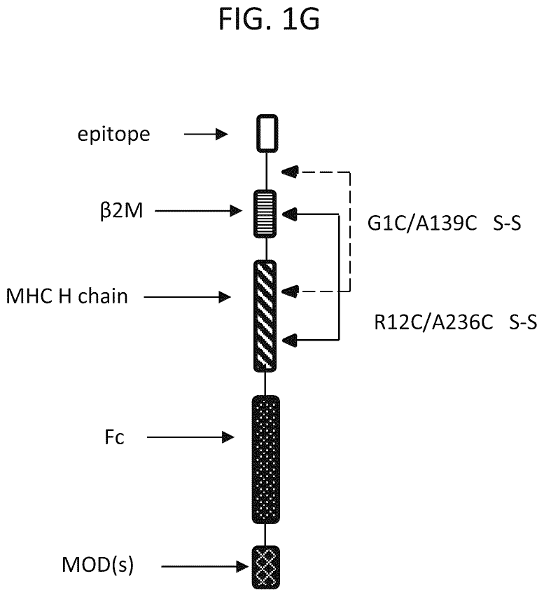

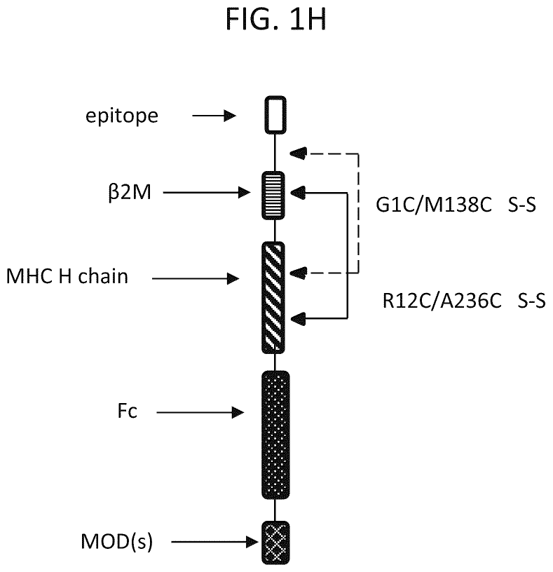

As noted above, a TMP comprises a pMHC polypeptide. A pMHC polypeptide comprises: a) a KRAS peptide having a length of from 4 amino acids to 25 amino acids (e.g., from 8 amino acids to 12 amino acids); b) a first peptide linker, where the first peptide linker comprise a cysteine (Cys) residue; c) a beta-2 microglobulin (β2M) polypeptide; d) a second peptide linker; and e) a major histocompatibility complex (MHC) class I heavy chain polypeptide. The MHC class I heavy chain polypeptide comprises: i) a Cys at any one of amino acids 135-143, based on the numbering of the MHC class I heavy chain polypeptide depicted in FIG. 3A; and ii) an amino acid other than Cys at position 84, based on the numbering of the MHC class I heavy chain polypeptide depicted in FIG. 3A. The pMHC polypeptide comprises an intrachain disulfide bond formed between the Cys present in the first peptide linker and the Cys at any one of amino acids 135-143 of the MHC class I heavy chain polypeptide. The pMHC polypeptide presents the epitope for binding to a T cell receptor (TCR). FIG. 1A-1J provide schematic depictions of non-limiting examples of configurations of pMHC polypeptides of the present disclosure. In some cases, the pMHC polypeptide comprises, in order from N-terminus to C-terminus: a) a KRAS peptide having a length of from 4 amino acids to 25 amino acids; b) a first peptide linker, where the first peptide linker comprise a Cys residue; c) a β2M polypeptide; d) a second peptide linker; and e) an MHC class I heavy chain polypeptide (where the MHC class I heavy chain polypeptide comprises: i) a Cys at any one of amino acids 135-143, based on the numbering of the MHC class I heavy chain polypeptide depicted in FIG. 3A; and ii) an amino acid other than Cys at position 84, based on the numbering of the MHC class I heavy chain polypeptide depicted in FIG. 3A).

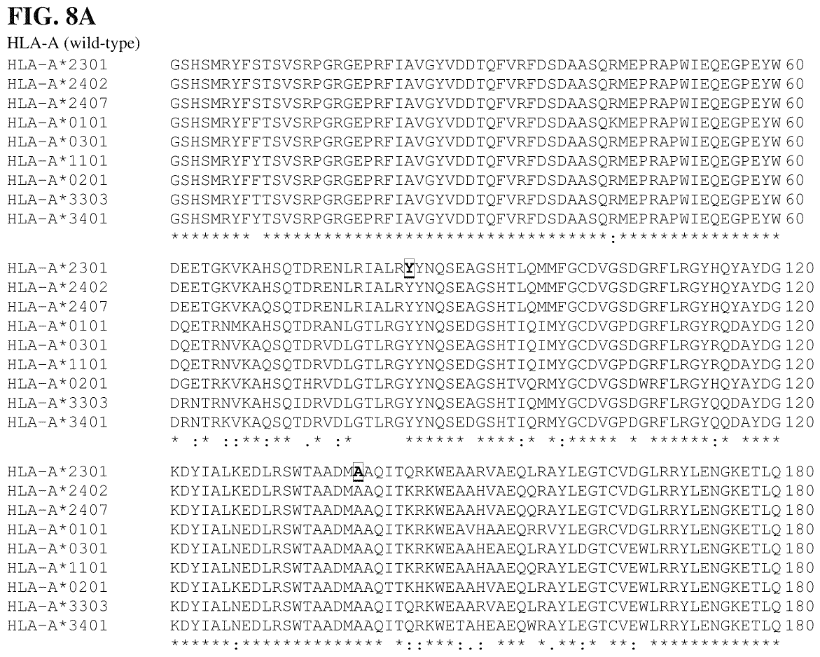

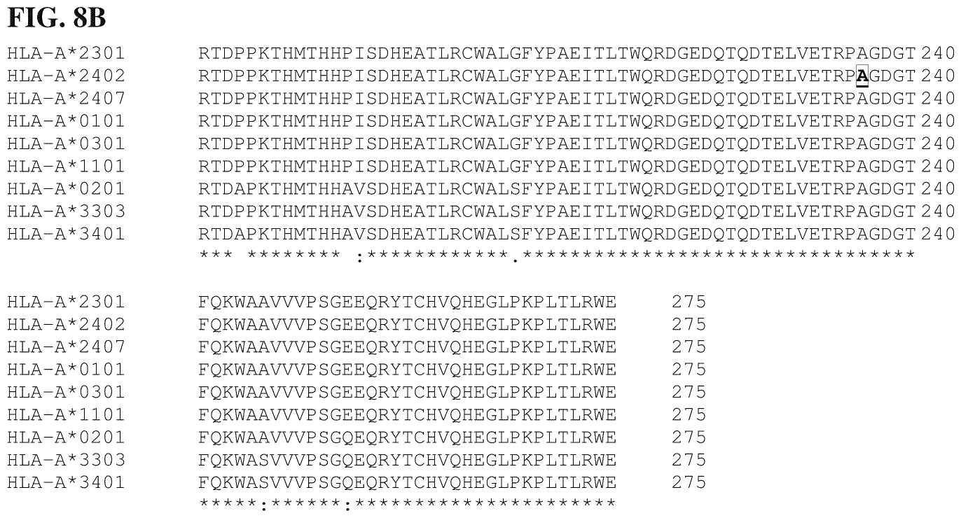

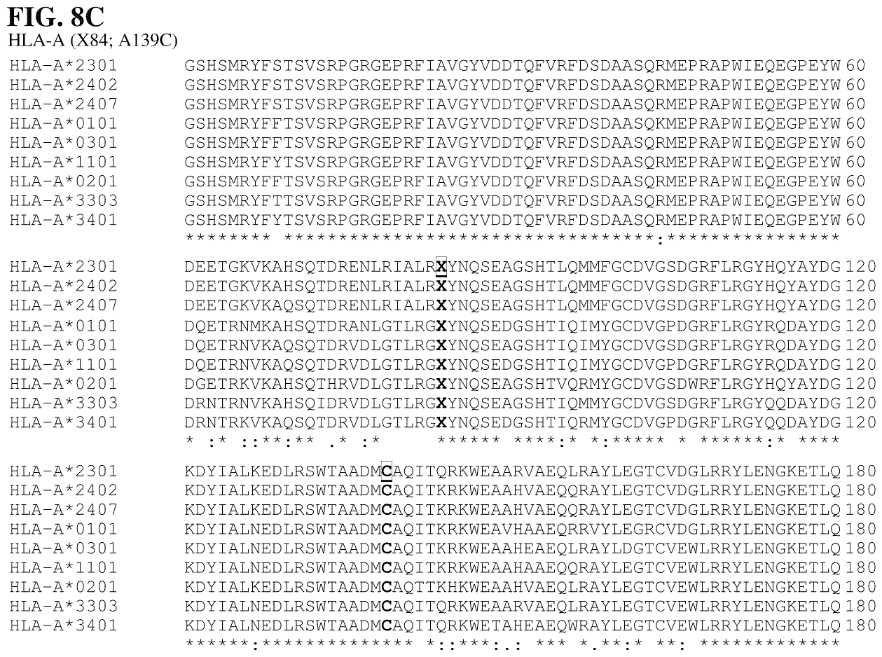

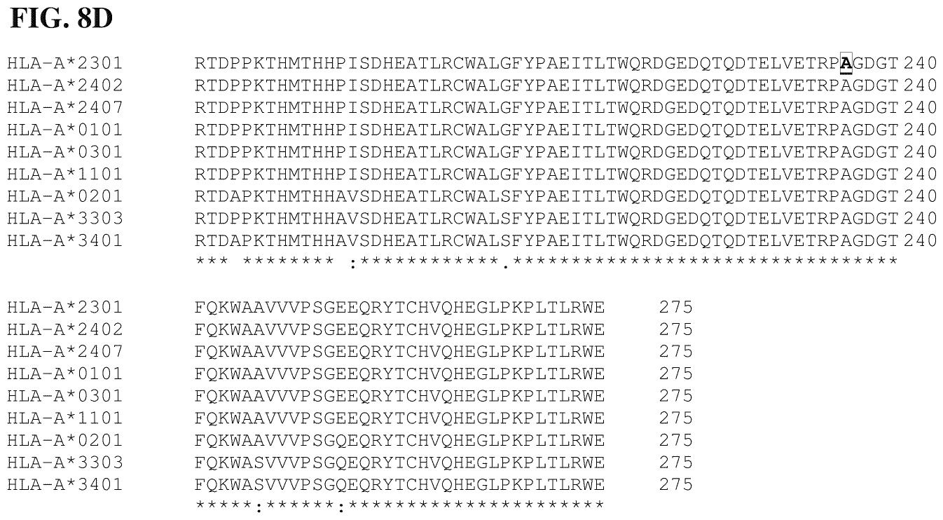

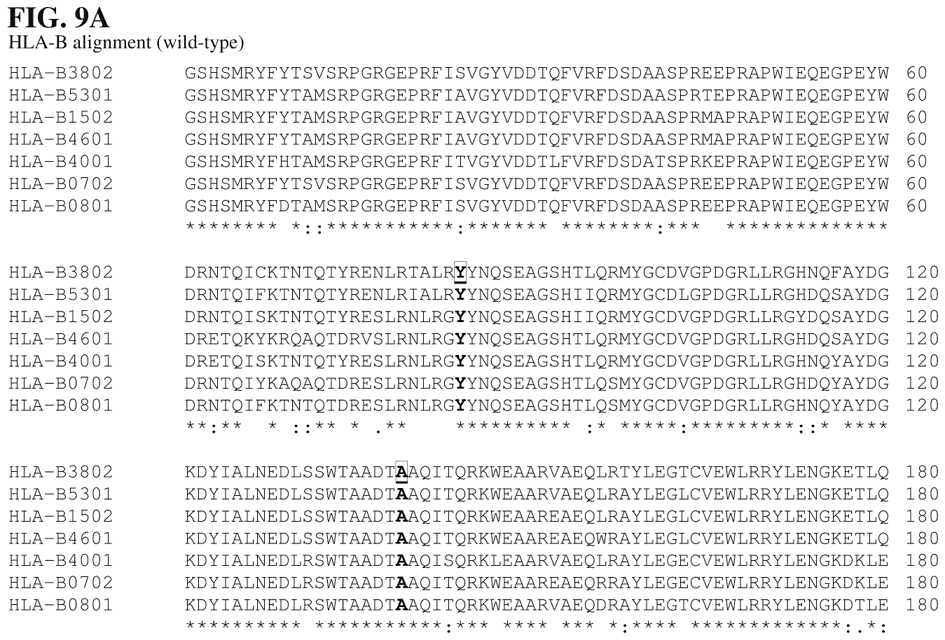

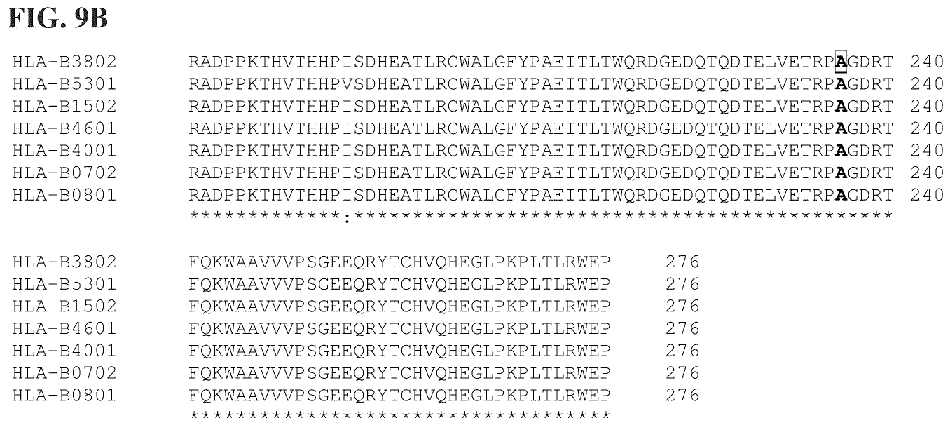

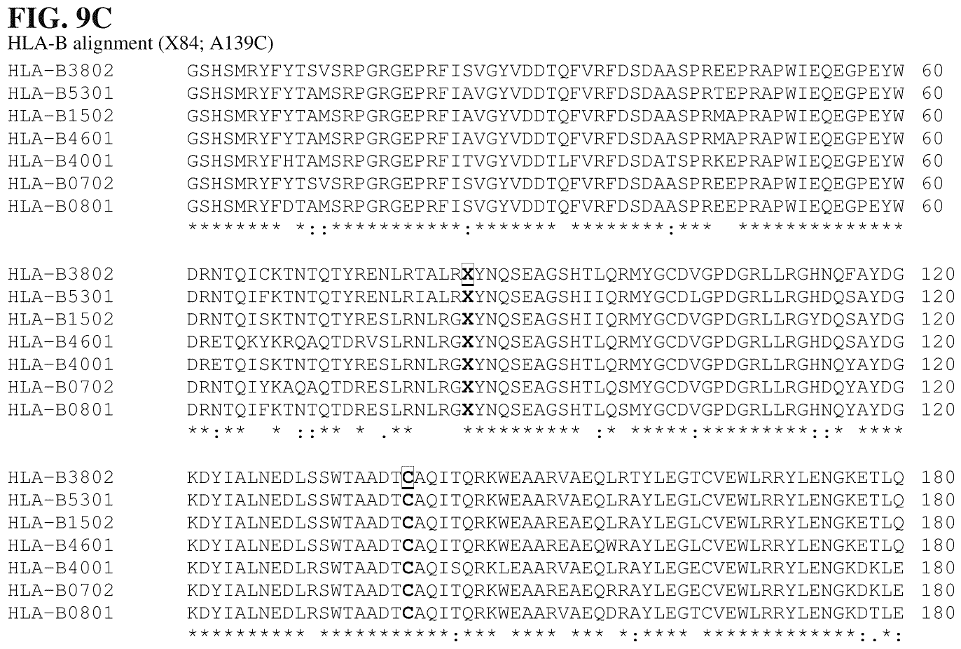

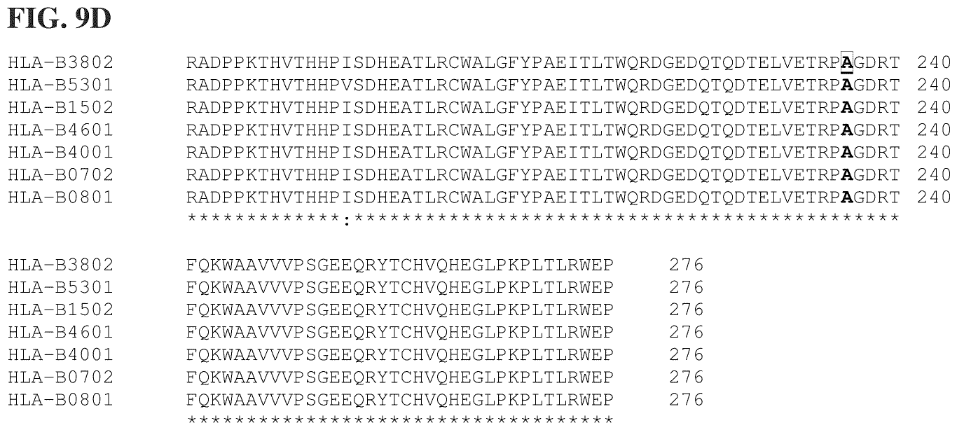

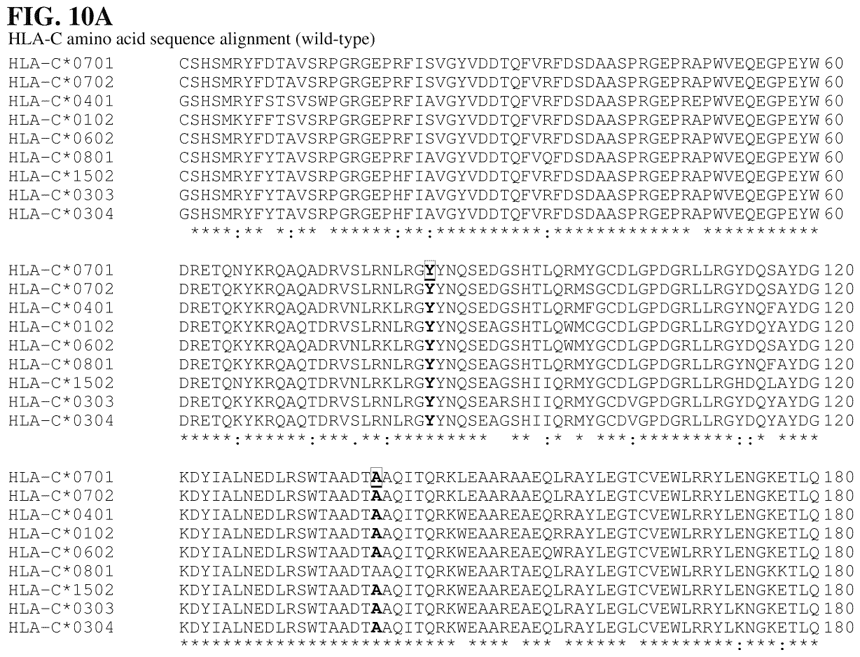

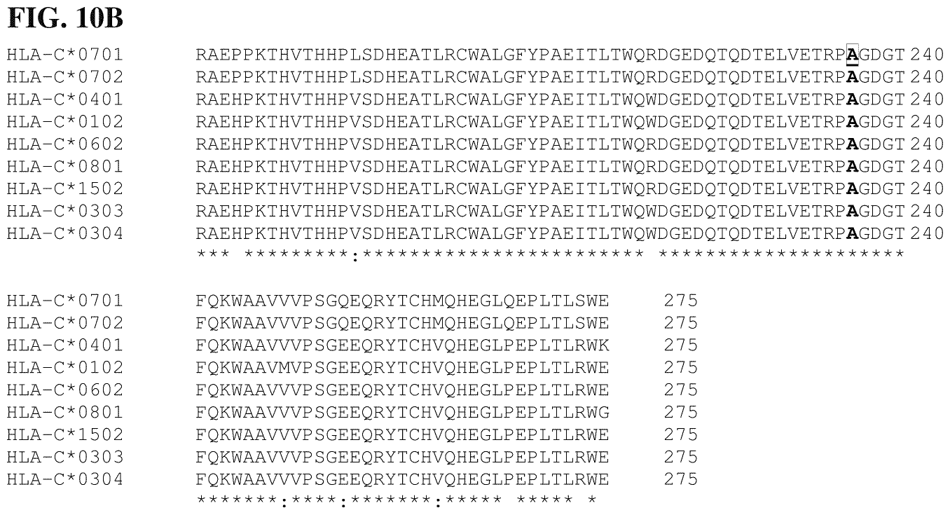

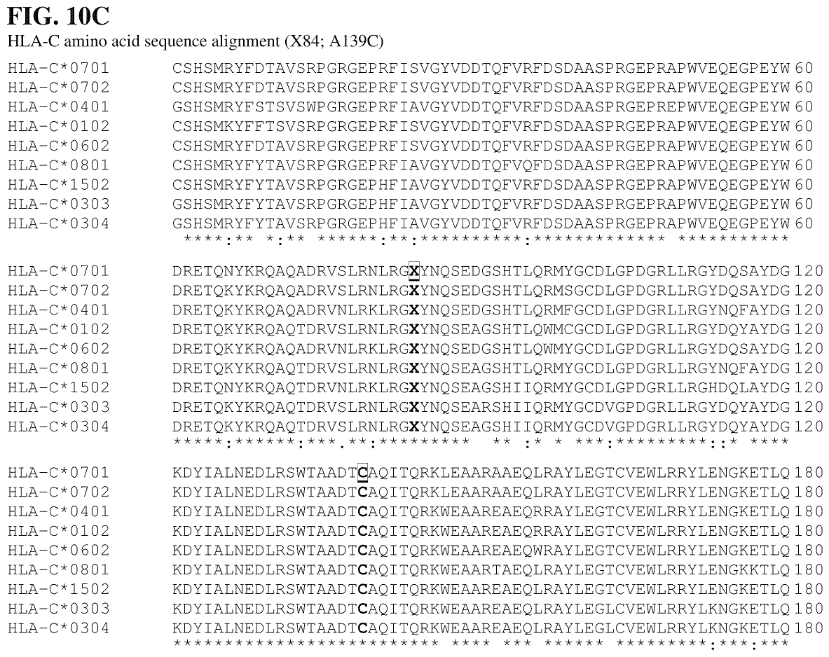

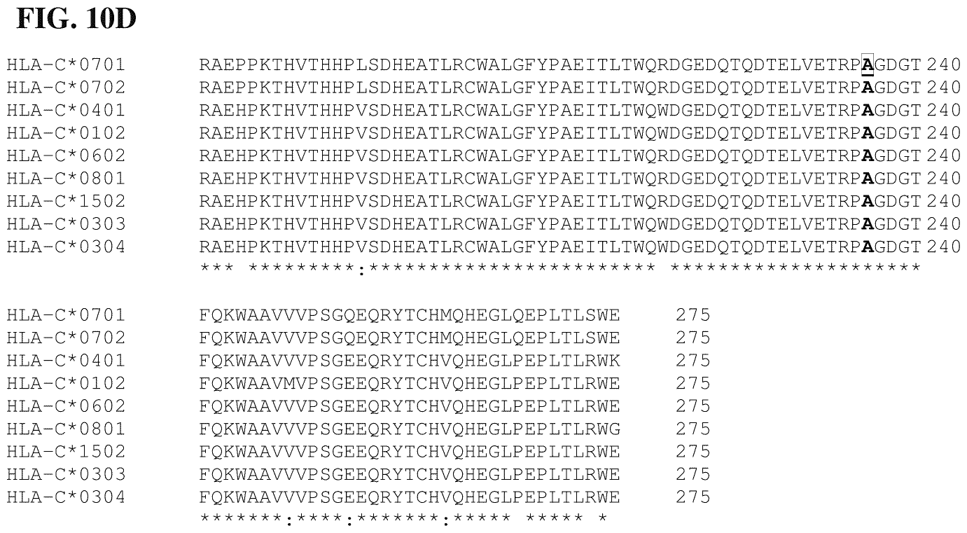

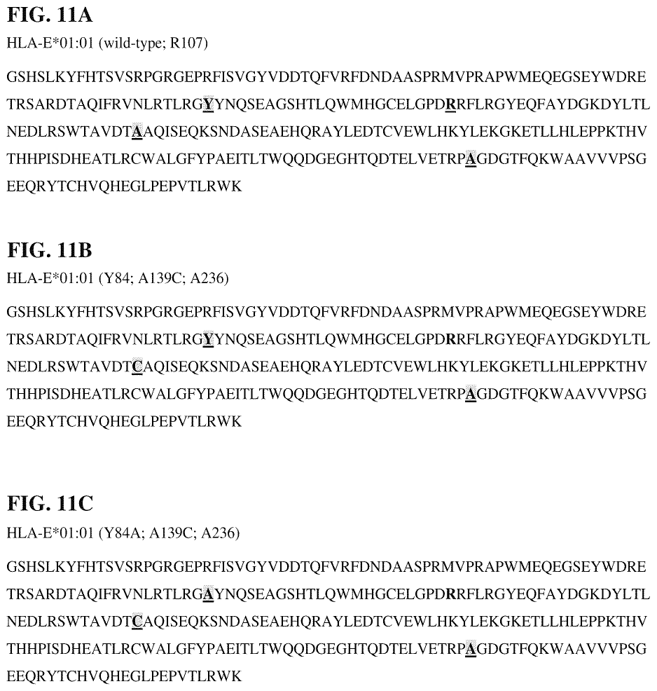

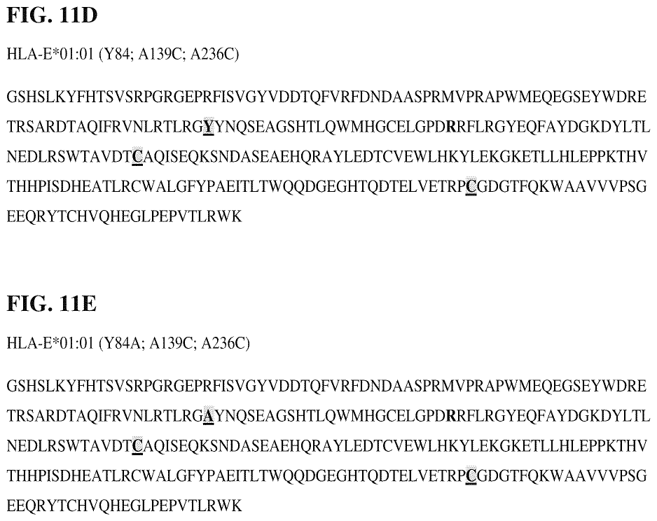

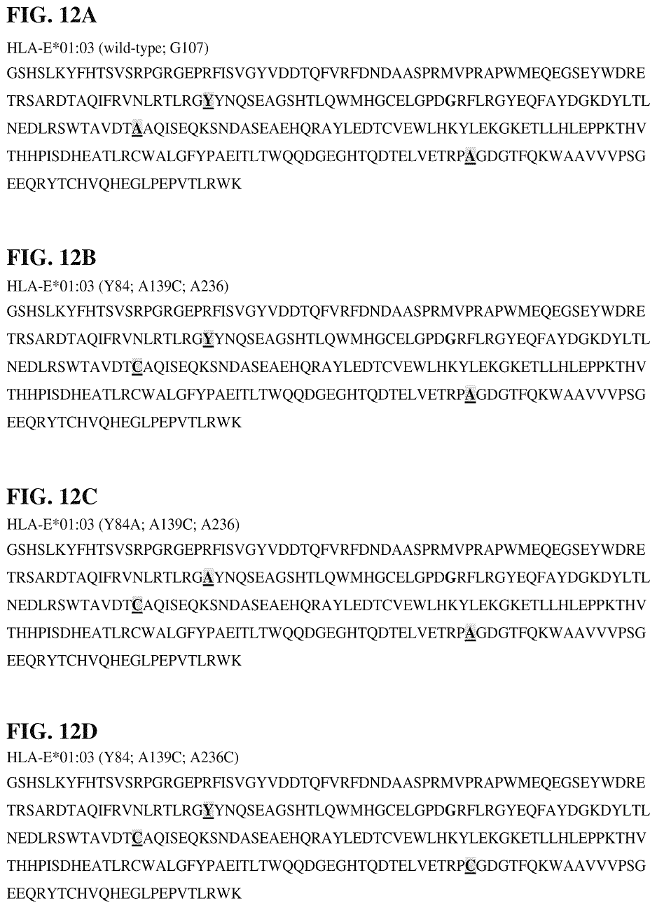

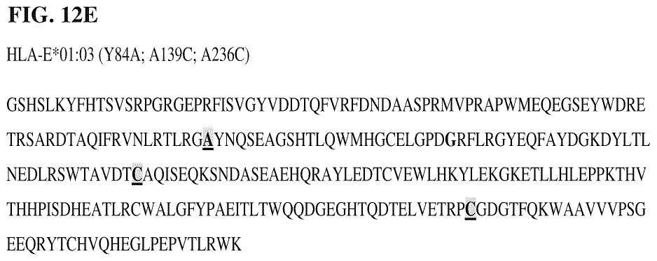

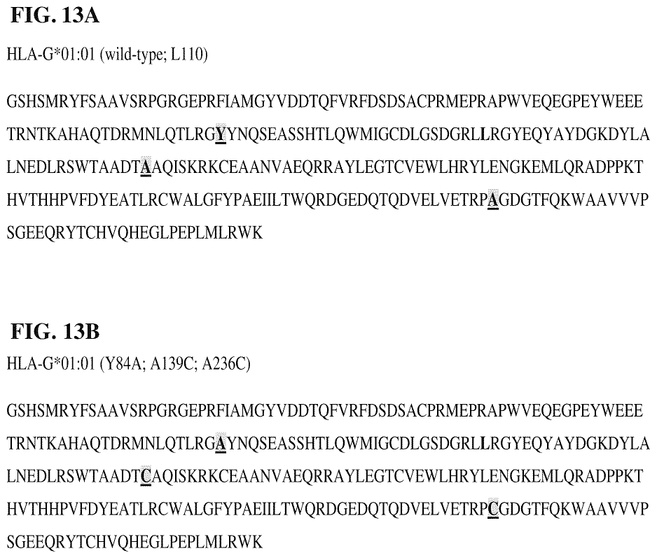

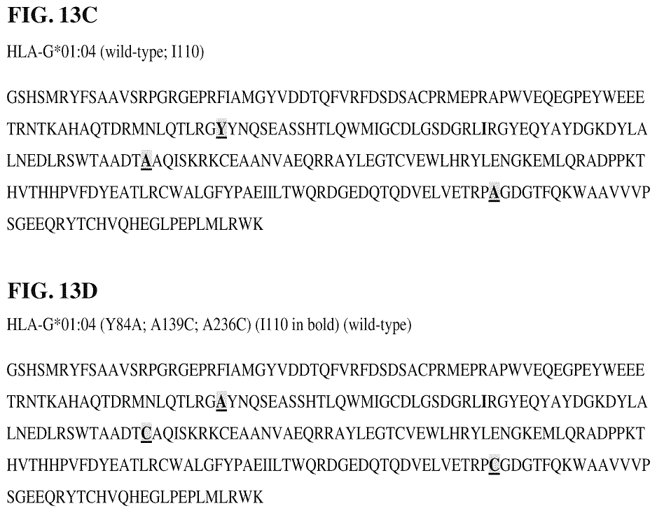

Unless otherwise specified below, the amino acid numbering of an MHC class I heavy chain polypeptide present in a pMHC polypeptide is based on the amino acid numbering of the MHC class I heavy chain polypeptide depicted in FIG. 3A. It should be noted that the amino acid numbering of the MHC class I heavy chain polypeptide depicted in FIG. 3A applies equally to the amino acid numbering of the MHC class I heavy chain polypeptides depicted in FIG. 8 (alignment of amino acid sequences of wild-type HLA-A heavy chain polypeptides), FIG. 9 (alignment of amino acid sequences of wild-type HLA-B heavy chain polypeptides), FIG. 10 (alignment of amino acid sequences of wild-type HLA-C heavy chain polypeptides), FIG. 11A and FIG. 12A (amino acid sequences of wild-type HLA-E heavy chain polypeptides), and FIGS. 13A and 13C (amino acid sequences of wild-type HLA-G heavy chain polypeptides).

### MHC Class I Heavy Chain Polypeptides

As noted above, a pMHC polypeptide comprises an MHC class I heavy chain polypeptide. Suitable MHC class I heavy chain polypeptides include a human MHC class I heavy chain polypeptide, where human MHC polypeptides are also referred to as “human leukocyte antigen” (“HLA”) polypeptides. Class I HLA heavy chain polypeptides include HLA-A heavy chain polypeptides, HLA-B heavy chain polypeptides, HLA-C heavy chain polypeptides, HLA-E heavy chain polypeptides, HLA-F heavy chain polypeptides, and HLA-G heavy chain polypeptides.

Unless expressly stated otherwise, pMHC polypeptide does not include membrane anchoring domains (transmembrane regions) of an MHC class I heavy chain, or a part of MHC class I heavy chain sufficient to anchor the resulting pMHC to a cell (e.g., eukaryotic cell such as a mammalian cell) in which it is expressed. In some cases, the MHC class I heavy chain present in a pMHC does not include a signal peptide, a transmembrane domain, or an intracellular domain (cytoplasmic tail) associated with a native MHC class I heavy chain. Thus, e.g., in some cases, the MHC class I heavy chain present in a pMHC polypeptide includes only the α1, α2, and α3 domains of an MHC class I heavy chain. In some cases, the MHC class I heavy chain present in a pMHC has a length of from about 270 amino acids (aa) to about 290 aa. In some cases, the MHC class I heavy chain present in a pMHC has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, an MHC class I heavy chain polypeptide present in a pMHC polypeptide comprises an amino acid sequence having at least 75%, at least 80%, at least 85%, at least 90%, at least 95%, at least 97%, at least 98%, at least 99%, or 100%, amino acid sequence identity to all or part (e.g., 50, 75, 100, 150, 200, or 250 contiguous amino acids) of the amino acid sequence of any of the human HLA heavy chain polypeptides depicted in FIGS. 3-13, where the MHC class I heavy chain polypeptide comprises: i) a Cys at any one of amino acids 135-143; and ii) an amino acid other than Cys at position 84. In some cases, the MHC class I heavy chain has a length of 270 aa, 271 aa, 272 aa, 273 aa, 274 aa, 275 aa, 276 aa, 277 aa, 278 aa, 279 aa, 280 aa, 281 aa, 282 aa, 283 aa, 284 aa, 285 aa, 286 aa, 287 aa, 288 aa, 289 aa, or 290 aa.

In some cases, a pMHC polypeptide comprises an HLA-A heavy chain polypeptide with the above-noted amino acid substitution(s). The HLA-A heavy chain polypeptides, or portions thereof, that may be that may be incorporated into a pMHC polypeptide include, but are not limited to, the alleles: A*0101, A*0201, A*0301, A*1101, A*2301, A*2402, A*2407, A*3303, and A*3401. In some cases, a pMHC polypeptide comprises an HLA-B heavy chain polypeptide with the above-noted amino acid substitution(s). In some cases, a pMHC polypeptide comprises an HLA-C heavy chain polypeptide with the above-noted amino acid substitution(s). In some cases, a pMHC polypeptide comprises an HLA-E heavy chain polypeptide with the above-noted amino acid substitution(s).

### Intrachain Disulfide Bonds

A TMP of the present disclosure comprises one or more intrachain disulfide bonds. A pMHC polypeptide present in a TMP comprises one intrachain disulfide bond formed between: i) a Cys at any one of amino acids 135-143, based on the numbering of the MHC class I heavy chain polypeptide depicted in FIG. 3A; and a Cys present in the first peptide linker, where the first peptide linker is between the KRAS peptide and the β2M polypeptide. A Cys is substituted for one of amino acids 135-143, in the peptide binding groove of the MHC class I heavy chain polypeptide. In some cases, the MHC class I heavy chain polypeptide in a pMHC polypeptide comprises a Cys at position 135. In some cases, the MHC class I heavy chain polypeptide in a pMHC polypeptide comprises a Cys at position 136. In some cases, the MHC class I heavy chain polypeptide in a pMHC polypeptide comprises a Cys at position 137. In some cases, the MHC class I heavy chain polypeptide in a pMHC polypeptide comprises a Cys at position 138. In some cases, the MHC class I heavy chain polypeptide in a pMHC polypeptide comprises a Cys at position 139. In some cases, the MHC class I heavy chain polypeptide in a pMHC polypeptide comprises a Cys at position 140. In some cases, the MHC class I heavy chain polypeptide in a pMHC polypeptide comprises a Cys at position 141. In some cases, the MHC class I heavy chain polypeptide in a pMHC polypeptide comprises a Cys at position 142. In some cases, the MHC class I heavy chain polypeptide in a pMHC polypeptide comprises a Cys at position 143.

Thus, for example, in some cases, amino acids 135-143 of the MHC class I heavy chain of a pMHC polypeptide comprises the amino acid sequence CADMAAQTT (SEQ ID NO: 196). As another example, in some cases, amino acids 135-143 of the MHC class I heavy chain of a pMHC polypeptide comprises the amino acid sequence ACDMAAQTT (SEQ ID NO: 197). As another example, in some cases, amino acids 135-143 of the MHC class I heavy chain of a pMHC polypeptide comprises the amino acid sequence AACMAAQTT (SEQ ID NO: 198). As another example, in some cases, amino acids 135-143 of the MHC class I heavy chain of a pMHC polypeptide comprises the amino acid sequence AADCAAQTT (SEQ ID NO: 199). As another example, in some cases, amino acids 135-143 of the MHC class I heavy chain of a pMHC polypeptide comprises the amino acid sequence AADMCAQTT (SEQ ID NO:200). As another example, in some cases, amino acids 135-143 of the MHC class I heavy chain of a pMHC polypeptide comprises the amino acid sequence AADMACQTT (SEQ ID NO:201). As another example, in some cases, amino acids 135-143 of the MHC class I heavy chain of a pMHC polypeptide comprises the amino acid sequence AADMAACTT (SEQ ID NO:202). As another example, in some cases, amino acids 135-143 of the MHC class I heavy chain of a pMHC polypeptide comprises the amino acid sequence AADMAAQCT (SEQ ID NO:203). As another example, in some cases, amino acids 135-143 of the MHC class I heavy chain of a pMHC polypeptide comprises the amino acid sequence AADMAAQTC (SEQ ID NO:204).

As another example, in some cases, amino acids 135-143 of the MHC class I heavy chain of a pMHC polypeptide comprises the amino acid sequence CADMAAQIT (SEQ ID NO:205). As another example, in some cases, amino acids 135-143 of the MHC class I heavy chain of a pMHC polypeptide comprises the amino acid sequence ACDMAAQIT (SEQ ID NO:206). As another example, in some cases, amino acids 135-143 of the MHC class I heavy chain of a pMHC polypeptide comprises the amino acid sequence AACMAAQIT (SEQ ID NO:207). As another example, in some cases, amino acids 135-143 of the MHC class I heavy chain of a pMHC polypeptide comprises the amino acid sequence AADCAAQIT (SEQ ID NO:208). As another example, in some cases, amino acids 135-143 of the MHC class I heavy chain of a pMHC polypeptide comprises the amino acid sequence AADMCAQIT (SEQ ID NO:209). As another example, in some cases, amino acids 135-143 of the MHC class I heavy chain of a pMHC polypeptide comprises the amino acid sequence AADMACQIT (SEQ ID NO:210). As another example, in some cases, amino acids 135-143 of the MHC class I heavy chain of a pMHC polypeptide comprises the amino acid sequence AADMAACIT (SEQ ID NO:211). As another example, in some cases, amino acids 135-143 of the MHC class I heavy chain of a pMHC polypeptide comprises the amino acid sequence AADMAAQCT (SEQ ID NO:212). As another example, in some cases, amino acids 135-143 of the MHC class I heavy chain of a pMHC polypeptide comprises the amino acid sequence AADMAAQIC (SEQ ID NO:213).

As another example, in some cases, amino acids 135-143 of the MHC class I heavy chain of a pMHC polypeptide comprises the amino acid sequence CADTAAQIT (SEQ ID NO:214). As another example, in some cases, amino acids 135-143 of the MHC class I heavy chain of a pMHC polypeptide comprises the amino acid sequence ACDTAAQIT (SEQ ID NO:215). As another example, in some cases, amino acids 135-143 of the MHC class I heavy chain of a pMHC polypeptide comprises the amino acid sequence AACTAAQIT (SEQ ID NO:216). As another example, in some cases, amino acids 135-143 of the MHC class I heavy chain of a pMHC polypeptide comprises the amino acid sequence AADCAAQIT (SEQ ID NO:217). As another example, in some cases, amino acids 135-143 of the MHC class I heavy chain of a pMHC polypeptide comprises the amino acid sequence AADTCAQIT (SEQ ID NO:218). As another example, in some cases, amino acids 135-143 of the MHC class I heavy chain of a pMHC polypeptide comprises the amino acid sequence AADTACQIT (SEQ ID NO:219). As another example, in some cases, amino acids 135-143 of the MHC class I heavy chain of a pMHC polypeptide comprises the amino acid sequence AADTAACIT (SEQ ID NO:220). As another example, in some cases, amino acids 135-143 of the MHC class I heavy chain of a pMHC polypeptide comprises the amino acid sequence AADTAAQCT (SEQ ID NO:221). As another example, in some cases, amino acids 135-143 of the MHC class I heavy chain of a pMHC polypeptide comprises the amino acid sequence AADTAAQIC (SEQ ID NO:222).

As another example, in some cases, amino acids 135-143 of the MHC class I heavy chain of a pMHC polypeptide comprises the amino acid sequence CVDTAAQIS (SEQ ID NO:223). As another example, in some cases, amino acids 135-143 of the MHC class I heavy chain of a pMHC polypeptide comprises the amino acid sequence ACDTAAQIS (SEQ ID NO:224). As another example, in some cases, amino acids 135-143 of the MHC class I heavy chain of a pMHC polypeptide comprises the amino acid sequence AVCTAAQIS (SEQ ID NO:225). As another example, in some cases, amino acids 135-143 of the MHC class I heavy chain of a pMHC polypeptide comprises the amino acid sequence AVDCAAQIS (SEQ ID NO:226). As another example, in some cases, amino acids 135-143 of the MHC class I heavy chain of a pMHC polypeptide comprises the amino acid sequence AVDTCAQIS (SEQ ID NO:227). As another example, in some cases, amino acids 135-143 of the MHC class I heavy chain of a pMHC polypeptide comprises the amino acid sequence AVDTACQIS (SEQ ID NO:228). As another example, in some cases, amino acids 135-143 of the MHC class I heavy chain of a pMHC polypeptide comprises the amino acid sequence AVDTAACIS (SEQ ID NO:229). As another example, in some cases, amino acids 135-143 of the MHC class I heavy chain of a pMHC polypeptide comprises the amino acid sequence AVDTAAQCS (SEQ ID NO:230). As another example, in some cases, amino acids 135-143 of the MHC class I heavy chain of a pMHC polypeptide comprises the amino acid sequence AVDTAAQIC (SEQ ID NO:231).

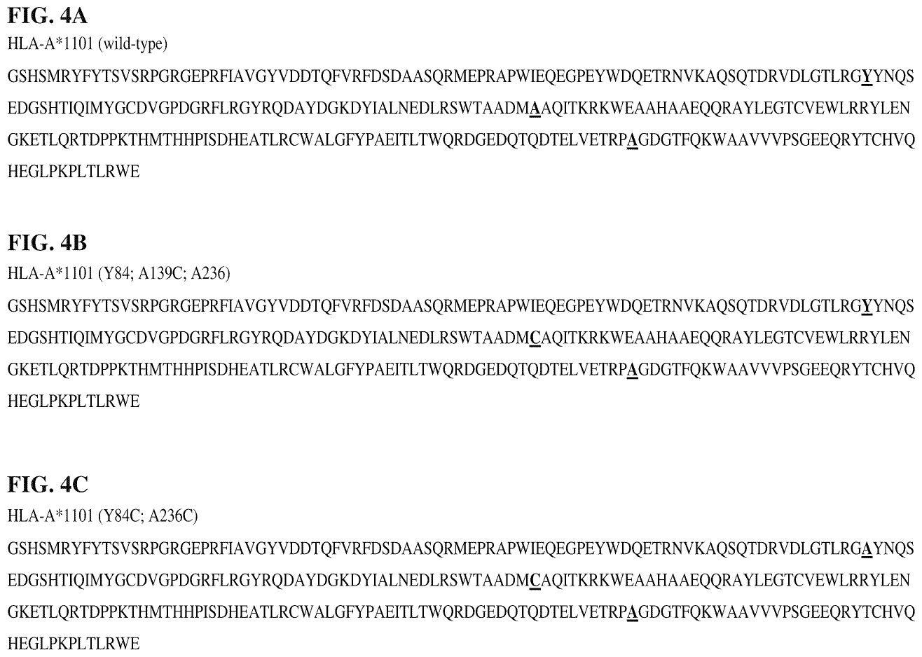

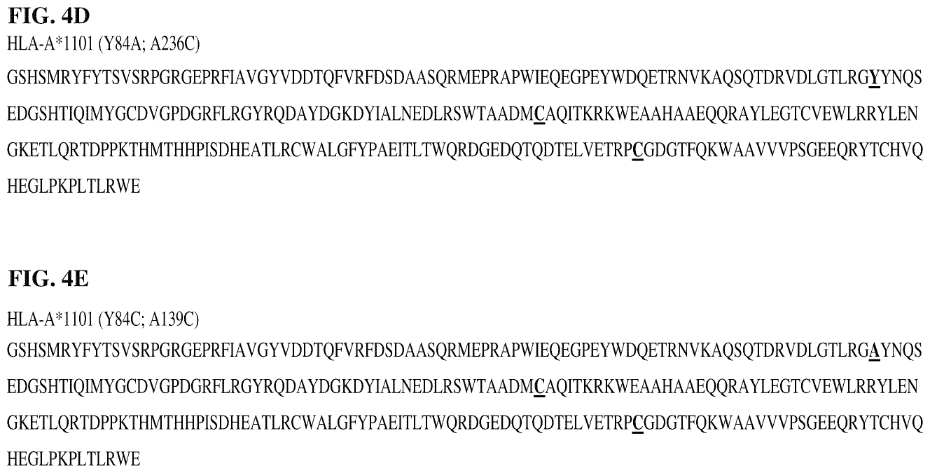

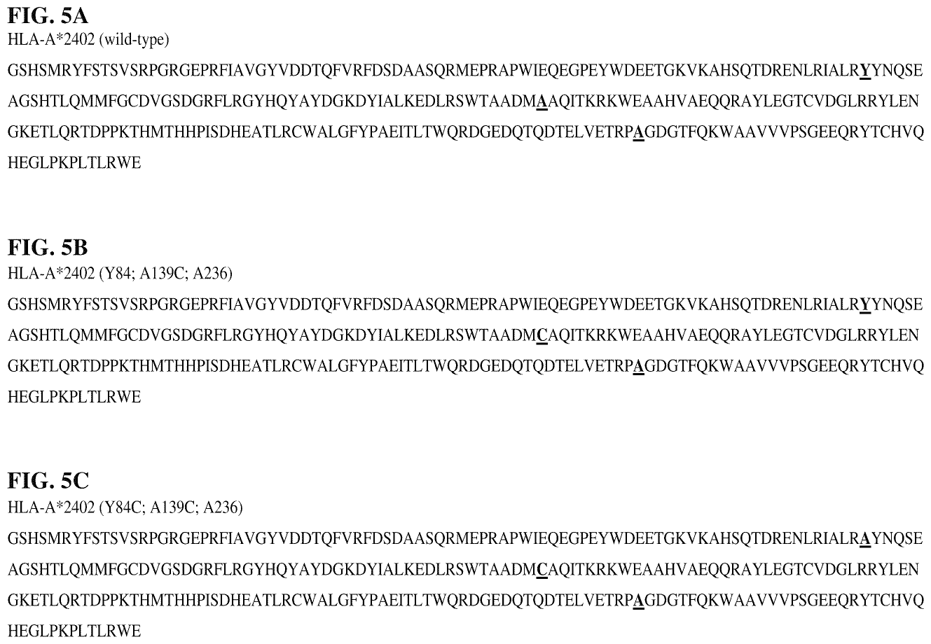

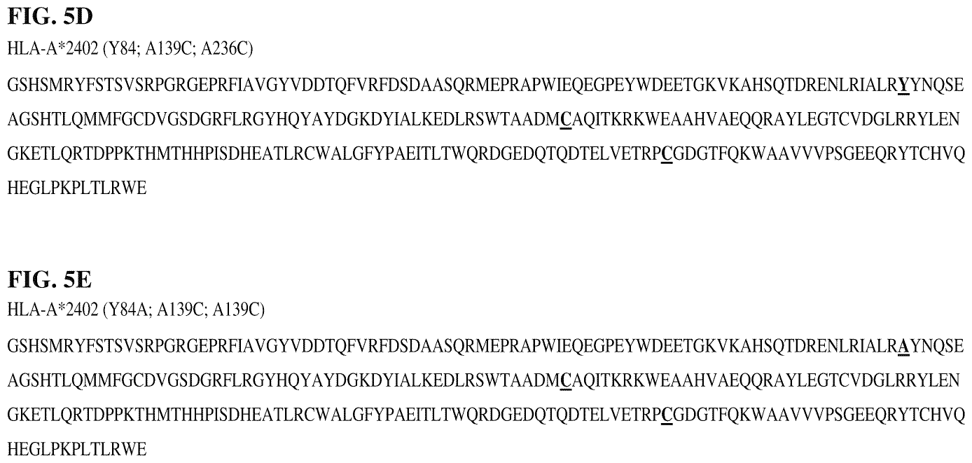

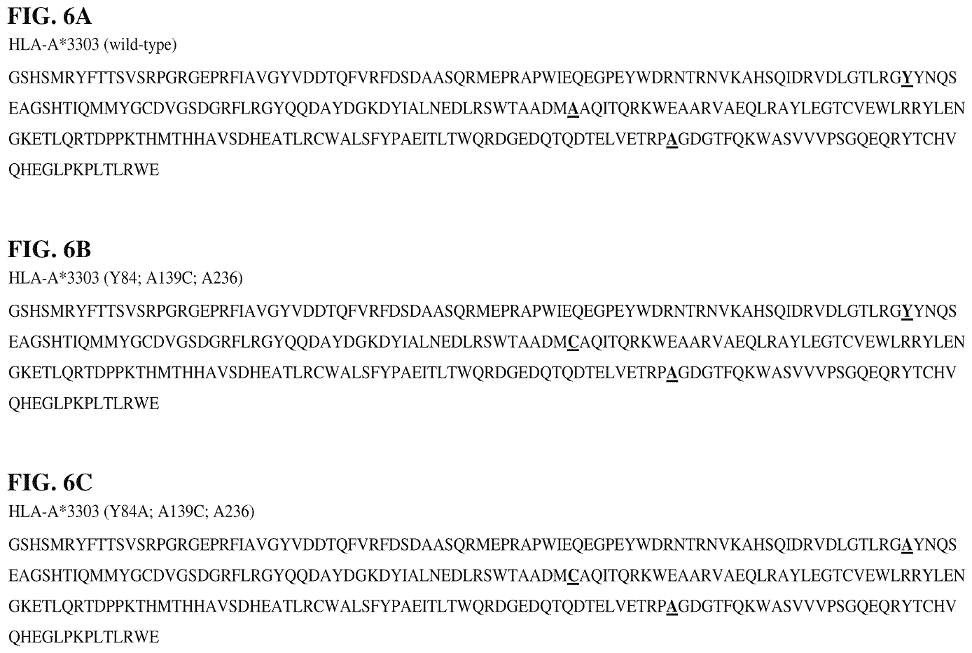

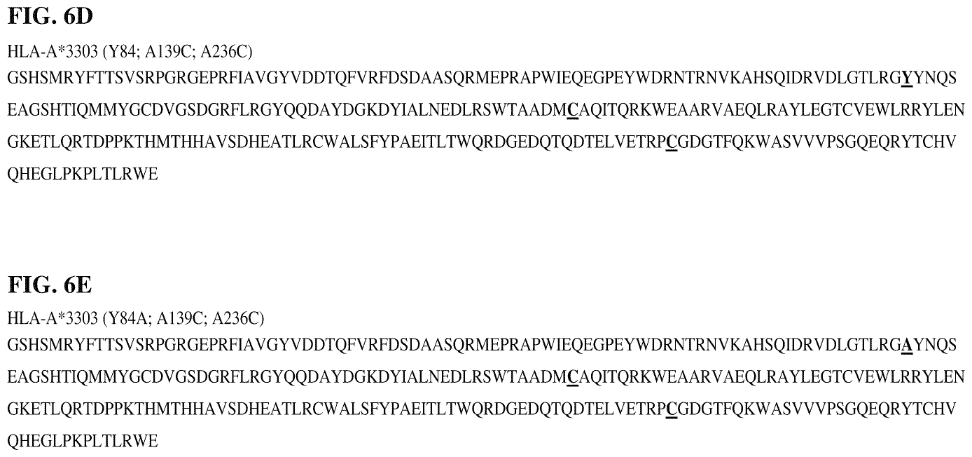

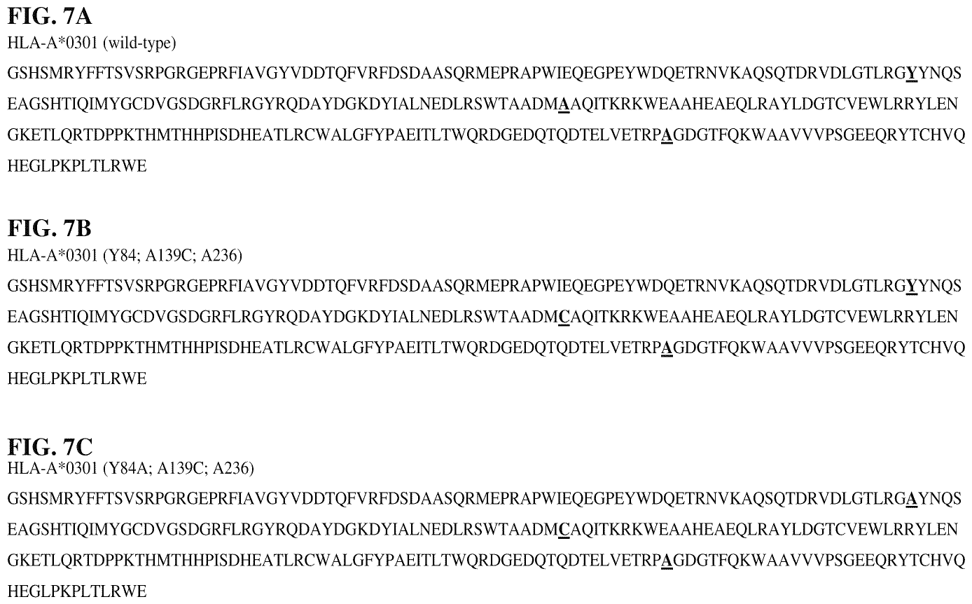

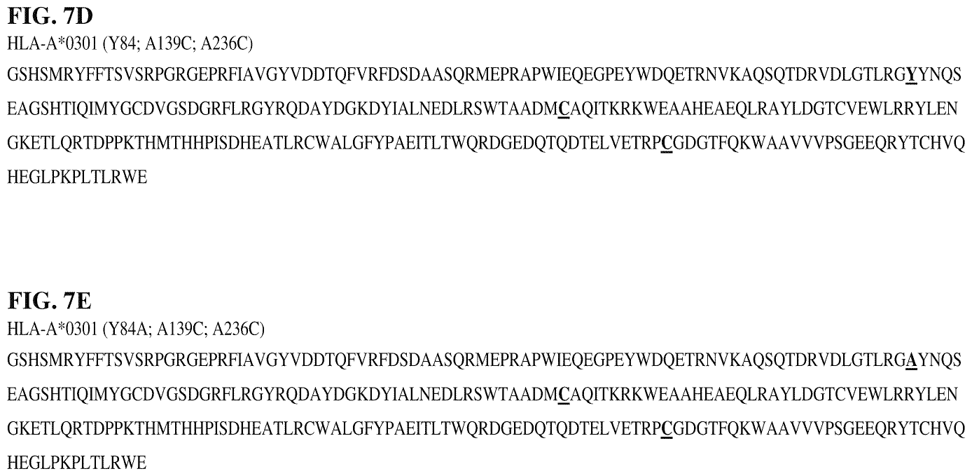

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-A amino acid sequences depicted in FIG. 8C-8D, where amino acid 139 is Cys, where amino acid 84 is Ala, and where amino acid 236 is Cys. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to any one of the HLA-A amino acid sequence depicted in FIG. 3B-3E, FIG. 4B-4E, FIG. 5B-5E, FIG. 6B-6E, and FIG. 7B-7E.

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-B amino acid sequences depicted in FIG. 9C-9D, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, where amino acid 84 is Ala, and where amino acid 236 is Ala. In some cases, the MHC class I heavy chain polypeptide in a pMHC polypeptide comprises an amino acid sequence having at least 85%, at least 90%, at least 95%, at least 97%, at least 98%, or at least 99%, amino acid sequence identity to any one of the HLA-C amino acid sequences depicted in FIG. 10C-10D, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Ala, and where amino acid 236 is Ala. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to any one of the HLA-E amino acid sequences depicted in FIGS. 11B-11E and 12B-12E, where amino acid 139 is Cys, where amino acid 84 is Ala, and where amino acid 236 is Cys.

In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, and where amino acid 84 is other than a Cys (e.g., where amino acid 84 is Ala, Gly, or Val). In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to amino acid sequence identity to the HLA-G amino acid sequence depicted in FIG. 13B or FIG. 13D, where amino acid 139 is Cys, and where amino acid 84 is other than a Cys (e.g., where amino acid 84 is Tyr. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Tyr, and where amino acid 236 is Ala. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Tyr, and where amino acid 236 is Cys. In some cases, the MHC class I heavy chain polypeptide in a pMHC polypeptide comprises an amino acid sequence having at least 85%, at least 90%, at least 95%, at least 97%, at least 98%, at least 99%, or 100%, amino acid sequence identity to the HLA-G amino acid sequence depicted in FIG. 13B or FIG. 13D where amino acid 139 is Cys, where amino acid 84 is Ala, and where amino acid 236 is Ala. In some cases, the MHC class I heavy chain polypeptide in a pMHC polypeptide comprises an amino acid having at least 85%, at least 90%, at least 95%, at least 97%, at least 98%, at least 99%, or 100%, amino acid sequence identity to the HLA-G amino acid sequence depicted in FIG. 13B or FIG. 13D, where amino acid 139 is Cys, where amino acid 84 is Ala, and where amino acid 236 is Cys.

The first peptide linker in a pMHC polypeptide of the present disclosure comprises a single Cys, which forms an intrachain disulfide bond with a Cys at one of amino acids 135-143 of the MHC class I heavy chain polypeptide. The first peptide linker present in a pMHC polypeptide can have a length of from about 5 amino acids to about 50 amino acids, e.g., from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

In some cases, the first peptide linker comprises the amino acid sequence CGGGS(GGGGS)n (SEQ ID NO:232), GCGGS(GGGGS)n (SEQ ID NO:233), or GGCGS(GGGGS)n (SEQ ID NO:234), where n is an integer from 1-10 (i.e., where n is 1, 2, 3, 4, 5, 6, 7, 8, 9, or 10). In some cases, the first peptide linker comprises the amino acid sequence CGGGS(GGGGS)n (SEQ ID NO:232), where n is an integer from 1-10 (i.e., where n is 1, 2, 3, 4, 5, 6, 7, 8, 9, or 10); such a peptide linker is referred to as a “G1C” linker. In some cases, the first peptide linker comprises the amino acid sequence CGGGS(GGGGS)n (SEQ ID NO:235), where n is 1. In some cases, the first peptide linker comprises the amino acid sequence CGGGS(GGGGS)n (SEQ ID NO:236), where n is 2. In some cases, the first peptide linker comprises the amino acid sequence CGGGS(GGGGS)n (SEQ ID NO:237), where n is 3. In some cases, the first peptide linker comprises the amino acid sequence GCGGS(GGGGS)n (SEQ ID NO:233), where n is an integer from 1-10 (i.e., where n is 1, 2, 3, 4, 5, 6, 7, 8, 9, or 10); such a peptide linker is referred to as a “G2C” linker. In some cases, the first peptide linker comprises the amino acid sequence GCGGS(GGGGS)n (SEQ ID NO:238), where n is 1. In some cases, the first peptide linker comprises the amino acid sequence GCGGS(GGGGS)n (SEQ ID NO:239), where n is 2. In some cases, the first peptide linker comprises the amino acid sequence GCGGS(GGGGS)n (SEQ ID NO:240), where n is 3. In some cases, the first peptide linker comprises the amino acid sequence GGCGS(GGGGS)n (SEQ ID NO:234), where n is an integer from 1-10 (i.e., where n is 1, 2, 3, 4, 5, 6, 7, 8, 9, or 10); such a linker is referred to as a “G3C” linker. In some cases, the first peptide linker comprises the amino acid sequence GGCGS(GGGGS)n (SEQ ID NO:241), where n is 1. In some cases, the first peptide linker comprises the amino acid sequence GGCGS(GGGGS)n (SEQ ID NO:242), where n is 2. In some cases, the first peptide linker comprises the amino acid sequence GGCGS(GGGGS)n (SEQ ID NO:243), where n is 3.

### (G2C; A139C)

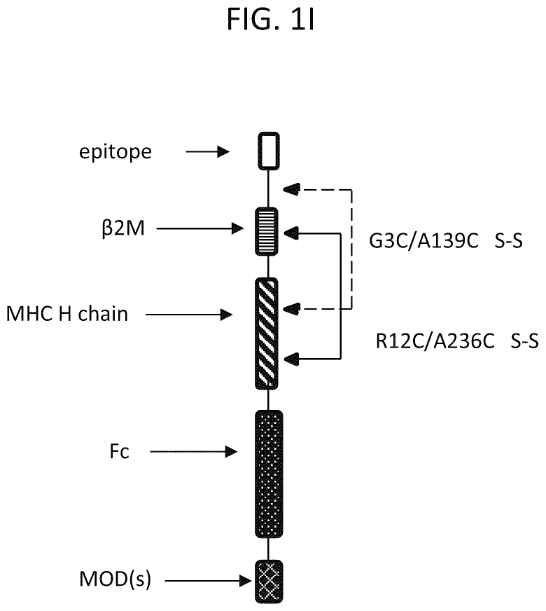

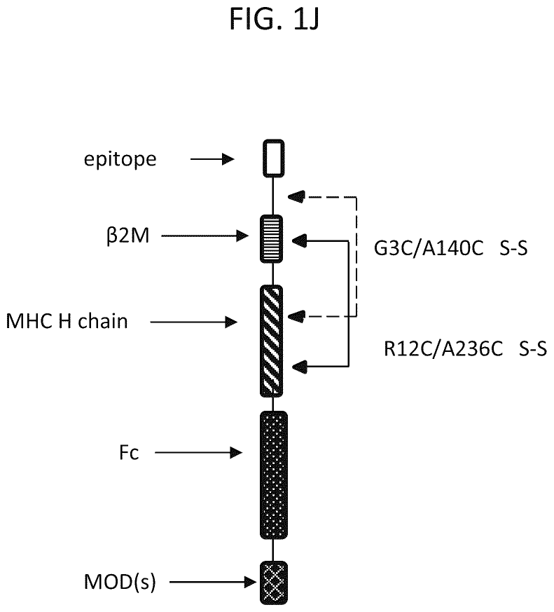

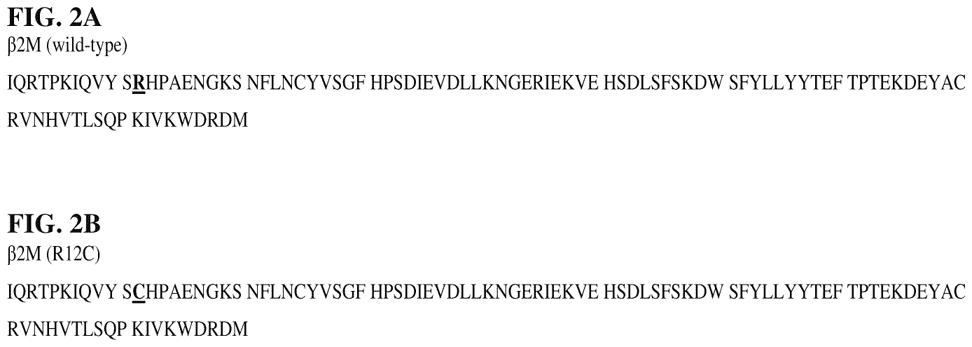

In some cases, a pMHC comprises an intrachain disulfide bond between: i) a Cys at amino acid 139 of the MHC class I heavy chain polypeptide; and ii) a Cys in the first peptide linker, where the peptide linker is a “G2C” linker and comprises the amino acid sequence GCGGS(GGGGS)n (SEQ ID NO:233), where n is an integer from 1 to 10. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:240), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1A provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. FIG. 1B provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G2C; M138C or T138C)

In some cases, a pMHC polypeptide comprises an intrachain disulfide bond between: i) a Cys at amino acid 138 of the MHC class I heavy chain polypeptide; and ii) a Cys in the first peptide linker, where the peptide linker is a “G2C” linker and comprises the amino acid sequence GCGGS(GGGGS)n (SEQ ID NO:233), where n is an integer from 1 to 10. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:240), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. In some cases, the pMHC polypeptide comprises only a single intrachain disulfide bond, where the single intrachain disulfide bond is between: i) a Cys at amino acid 138 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G2C; A140C)

In some cases, a pMHC polypeptide comprises an intrachain disulfide bond between: i) a Cys at amino acid 140 of the MHC class I heavy chain polypeptide; and ii) a Cys in the first peptide linker, where the peptide linker is a “G2C” linker and comprises the amino acid sequence GCGGS(GGGGS)n (SEQ ID NO:233), where n is an integer from 1 to 10. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:238), where n is 1. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:239), where n is 2. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:240), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. In some cases, the pMHC polypeptide comprises only a single intrachain disulfide bond, where the single intrachain disulfide bond is between: i) a Cys at amino acid 140 of the MHC class I heavy chain polypeptide; and ii) a Cys in the first peptide linker, where the peptide linker comprises the amino acid sequence GCGGS(GGGGS)n (SEQ ID NO:233), where n is an integer from 1 to 10. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G1C; A139C)

In some cases, a pMHC polypeptide comprises an intrachain disulfide bond between: i) a Cys at amino acid 139 of the MHC class I heavy chain polypeptide; and ii) a Cys in the first peptide linker, where the peptide linker is a “G1C” linker and comprises the amino acid sequence CGGGS(GGGGS)n (SEQ ID NO:232), where n is an integer from 1 to 10. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:235), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:236), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:237), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1G provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence CGGGS(GGGGS)n (SEQ ID NO:232), where n is an integer from 1 to 10. FIG. 1C provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G1C; M138C or T138C)

In some cases, a pMHC polypeptide comprises an intrachain disulfide bond between: i) a Cys at amino acid 138 of the MHC class I heavy chain polypeptide; and ii) a Cys in the first peptide linker, where the peptide linker is a “G1C” linker and comprises the amino acid sequence CGGGS(GGGGS)n (SEQ ID NO:232), where n is an integer from 1 to 10. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:235), where n is 1. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:236), where n is 2. In some cases, a pMHC polypeptide comprises an intrachain disulfide bond between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:237), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1H provides a schematic depiction of such a pMHC. In some cases, the pMHC polypeptide comprises only a single intrachain disulfide bond, where the single intrachain disulfide bond is between: i) a Cys at amino acid 138 of the MHC class I heavy chain polypeptide; and ii) a Cys in the first peptide linker, where the peptide linker comprises the amino acid sequence CGGGS(GGGGS)n (SEQ ID NO:232), where n is an integer from 1 to 10. FIG. 1D provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G3C; A139C)

In some cases, a pMHC polypeptide comprises an intrachain disulfide bond between: i) a Cys at amino acid 139 of the MHC class I heavy chain polypeptide; and ii) a Cys in the first peptide linker, where the peptide linker is a “G3C” linker and comprises the amino acid sequence GGCGS(GGGGS)n (SEQ ID NO:234), where n is an integer from 1 to 10. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:241), where n is 1. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:242), where n is 2. In some cases, a pMHC polypeptide comprises an intrachain disulfide bond between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:243), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1I provides a schematic depiction of such a pMHC. In some cases, the pMHC polypeptide comprises only a single intrachain disulfide bond, where the single intrachain disulfide bond is between: i) a Cys at amino acid 139 of the MHC class I heavy chain polypeptide; and ii) a Cys in the first peptide linker, where the peptide linker comprises the amino acid sequence GGCGS(GGGGS)n (SEQ ID NO:234), where n is an integer from 1 to 10. FIG. 1E provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

### (G3C; A140C)

In some cases, a pMHC polypeptide comprises an intrachain disulfide bond between: i) a Cys at amino acid 140 of the MHC class I heavy chain polypeptide; and ii) a Cys in the first peptide linker, where the peptide linker is a “G3C” linker and comprises the amino acid sequence GGCGS(GGGGS)n (SEQ ID NO:234), where n is an integer from 1 to 10. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:241), where n is 1. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:242), where n is 2. In some cases, a pMHC polypeptide comprises an intrachain disulfide bond between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:243), where n is 3. In some cases, the pMHC polypeptide comprises a second disulfide bond between: i) a Cys at amino acid position 12 of the β2M polypeptide, based on the numbering of the β2M amino acid sequence depicted in FIG. 2B; and ii) a Cys at amino acid 236 of the MHC class I heavy chain polypeptide. FIG. 1J provides a schematic depiction of such a pMHC. In some cases, the pMHC polypeptide comprises only a single intrachain disulfide bond, where the single intrachain disulfide bond is between: i) a Cys at amino acid 140 of the MHC class I heavy chain polypeptide; and ii) a Cys in the first peptide linker, where the peptide linker comprises the amino acid sequence GGCGS(GGGGS)n (SEQ ID NO:234), where n is an integer from 1 to 10. FIG. 1F provides a schematic depiction of such a pMHC. In any of the above embodiments, in some cases amino acid 84 of the pMHC polypeptide is Ala. In any of the above embodiments, in some cases amino acid 84 of the pMHC polypeptide is Gly. In any of the above embodiments, in some cases amino acid 84 of the pMHC polypeptide is Val. In any of the above embodiments, in some cases amino acid 84 of the pMHC polypeptide is Tyr.

The MHC class I heavy chain polypeptide present in a pMHC polypeptide comprises an amino acid other than Cys at position 84, based on the numbering of the MHC class I heavy chain polypeptide depicted in FIG. 3A. A wild-type MHC class I heavy chain polypeptide comprises a Tyr at position 84. In some cases, the MHC class I heavy chain polypeptide present in a pMHC polypeptide comprises a Tyr at position 84. Substitution of the Tyr at position 84 with an amino acid comprising a small, nonpolar side chain enhances access of the KRAS peptide to the peptide binding site of the β2M/MHC class I heavy chain polypeptide complex. In some cases, the MHC class I heavy chain polypeptide present in a pMHC polypeptide comprises an amino acid selected from Ala, Gly, and Val at position 84, based on the numbering of the MHC class I heavy chain polypeptide depicted in FIG. 3A. In some cases, the MHC class I heavy chain polypeptide present in a pMHC polypeptide comprises an Ala at position 84, based on the numbering of the MHC class I heavy chain polypeptide depicted in FIG. 3A. In some cases, the MHC class I heavy chain polypeptide present in a pMHC polypeptide comprises a Gly at position 84, based on the numbering of the MHC class I heavy chain polypeptide depicted in FIG. 3A. In some cases, the MHC class I heavy chain polypeptide present in a pMHC polypeptide comprises a Val at position 84, based on the numbering of the MHC class I heavy chain polypeptide depicted in FIG. 3A.

### β2M Polypeptides

In some cases, a β2M polypeptide present in a pMHC polypeptide of the present disclosure can have an amino acid sequence having at least 80%, at least 85%, at least 90%, at least 95%, at least 97%, at least 98%, at least 99%, or 100%, amino acid sequence identity to the β2M amino acid sequence depicted in FIG. 2A. In some cases, a β2M polypeptide present in a pMHC polypeptide of the present disclosure can have an amino acid sequence having at least 80%, at least 85%, at least 90%, at least 95%, at least 97%, at least 98%, at least 99%, or 100%, amino acid sequence identity to the β2M amino acid sequence depicted in FIG. 2B, where the β2M polypeptide comprises a Cys at position 12.

### KRAS Peptides

A TMP of this disclosure comprises a pMHC polypeptide that comprises a KRAS peptide that, together with the other components of the pMHC, presents a KRAS epitope to a T cell (i.e., to a TCR present on the surface of a T cell). As used herein, the term “KRAS peptide” means a peptide that presents a KRAS epitope to a TCR when the KRAS peptide is bound to an MHC complex. As used herein, the term “KRAS epitope” means an epitope found on a KRAS protein. As used herein, the terms “KRAS” and “KRAS protein” are synonymous and mean a protein having an amino acid sequence present in a KRAS protein (including a variant, e.g., a mutated form) that is associated with a human cancer, including but not limited to one of the following: (i) a KRAS4A polypeptide; (ii) a KRAS4B; and (iii) variants of (i) and (ii) that are associated with a human cancer, including, e.g., mutated forms. As used herein, the term “KRAS polypeptide” means a polypeptide having a sequence of amino acids found in all or a part of a KRAS protein, or where specified, a polypeptide having at least 75%, at least 80%, at least 85%, at least 90%, at least 95%, at least 97%, at least 98%, at least 99%, or 100% amino acid sequence identity to a sequence of amino acids found in all or a part of a KRAS protein or a variant that occurs in a human cancer, including, e.g., mutated forms.

KRAS (also known as “KRAS proto-oncogene, GTPase,” Kirsten rat sarcoma viral oncogene homolog,” and “K-Ras P21 protein”) is a GTPase that controls cell proliferation. When mutated, KRAS can fail to control cell proliferation, leading to cancer. In SEQ ID NO:1 and SEQ ID NO:2, below, amino acids G12, G13, T35, I36, E49, Q61, K127, and A156 are in bold and underlined; substitutions of one or more of these residues can be present in a cancer-associated form of a KRAS polypeptide, although substitutions of other amino acids may be associated with a cancer.

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:1).

A wild-type (normal; non-cancer-associated) KRAS polypeptide can have the following amino acid sequence: MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:2).

Mutated forms of KRAS are associated with certain cancers; and at least a portion of the mutated form of KRAS is present on the surface of certain cancer cells. See, e.g., Prior et al. (2012) Cancer Res. 72:2457; and Warren and Holt (2010) Human Immunology 71:245. As noted above, in SEQ ID NO:1 and SEQ ID NO:2, amino acids G12, G13, T35, I36, E49, Q61, K127, and A156 are in bold and underlined; substitutions of one or more of these residues can be present in a cancer-associated form of a KRAS polypeptide. A cancer-associated KRAS polypeptide can include one or more of: i) a substitution of G12 (e.g. G12C, G12V, G12S, G12A, G12R, G12F, or G12D); ii) a substitution of G13 (e.g. G13C, G13D, G13R, G13V, G13S, or G13A); iii) a substitution of T35 (e.g., T351); iv) a substitution of 136 (e.g., 136L or 136M); v) a substitution of E49 (e.g., E49K); vi) a substitution of Q61 (e.g. Q61H, Q61R, Q61P, Q61E, Q61K, Q61L, or Q61K); vii) a substitution of K117 (e.g., K117N); and viii) a substitution of A146 (e.g. A146T or A146V); where the amino acid numbering is as set out in SEQ ID NO:1 and SEQ ID NO:2. See, e.g., U.S. 2019/0194192. A cancer-associated KRAS polypeptide can include both (i) a substitution of G12 (e.g. G12C, G12V, G12S, G12A, G12R, G12F, or G12D) and (ii) a substitution of G13 (e.g. G13C, G13D, G13R, G13V, G13S, or G13A).

For example, a cancer-associated, mutated form of a KRAS polypeptide can have one or more amino acid substitutions compared to the amino acid sequence set forth in SEQ ID NO:1 or SEQ ID NO:2. In some cases, a cancer-associated, mutated form of a KRAS polypeptide has only a single amino acid substitution compared to the amino acid sequence set forth in SEQ ID NO:1 or SEQ ID NO:2. In some cases, a cancer-associated, mutated form of a KRAS polypeptide has only two amino acid substitutions compared to the amino acid sequence set forth in SEQ ID NO:1 or SEQ ID NO:2. In some cases, a cancer-associated, mutated form of a KRAS polypeptide has only three amino acid substitutions compared to the amino acid sequence set forth in SEQ ID NO:1 or SEQ ID NO:2. In some cases, a cancer-associated, mutated form of a KRAS polypeptide has only four amino acid substitutions compared to the amino acid sequence set forth in SEQ ID NO:1 or SEQ ID NO:2. In some cases, a cancer-associated, mutated form of a KRAS polypeptide has only five amino acid substitutions compared to the amino acid sequence set forth in SEQ ID NO:1 or SEQ ID NO:2.

For example, KRAS(G12D) (a KRAS polypeptide having a G-to-D substitution at amino acid position 12, based on the amino acid numbering set forth in SEQ ID NO: 1) is associated with pancreatic ductal adenocarcinoma (PDAC). KRAS(G12V) (a KRAS polypeptide having a G-to-V substitution at amino acid position 12, based on the amino acid numbering set forth in SEQ ID NO:1 or SEQ ID NO:2) is also associated with pancreatic cancer. KRAS(G12R) (a KRAS polypeptide having a G-to-R substitution at amino acid position 12, based on the amino acid numbering set forth in SEQ ID NO:1 or SEQ ID NO:2) is also associated with pancreatic cancer. See, e.g., Waters and Der (2018) Cold Spring Harb. Perspect. Med. 8:(9). pii: a031435. doi: 10.1101/cshperspect.a031435. As another example, KRAS(G12C) (a KRAS polypeptide having a G-to-C substitution at amino acid position 12, based on the amino acid numbering set forth in SEQ ID NO:1 or SEQ ID NO:2) is associated with lung cancer, e.g., non-small cell lung cancer. See, e.g., Román et al. (2018) Mol. Cancer 17:33. Other mutated forms of KRAS (e.g., G12A; G12C; G12D; G12R; G12S; G12V; G13A; G13C; G13D; G13R; G13S; G13V) are associated with various cancers; where such cancers include, e.g., bile duct carcinoma, gall bladder carcinoma, adenocarcinoma, rectal adenocarcinoma, endometrial carcinoma, hematopoietic neoplasms, and lung cancer. See, e.g., Prior et al. (20120 Cancer Res. 72:2457.

As another example, a cancer-associated, mutated form of a KRAS polypeptide can have an amino acid substitution at amino acid 61 of a KRAS polypeptide (e.g., a KRAS polypeptide having the amino acid sequence set forth in SEQ ID NO:1 or SEQ ID NO:2). For example, a cancer-associated, mutated form of a KRAS polypeptide can have an amino acid substitution such as Q61H, Q61L, Q61E, Q61R, or Q61K.

A KRAS peptide present in a pMHC polypeptide (where the pMHC polypeptide is present in a TMP) can have a deletion of an amino acid, compared to wild-type KRAS. A KRAS peptide present in a TMP can have both a substitution compared to wild-type KRAS and a deletion of an amino acid compared to wild-type KRAS. Wild-type KRAS amino acids 5-14 can be, e.g., KLVVVGAGGV (SEQ ID NO:3; KRAS 5-14; where G12 and G13 are underlined and in bold). A KRAS peptide can have a deletion of amino acid 7 (where the numbering is based on the amino acid sequence set forth in SEQ ID NO:1 or SEQ ID NO:2). A KRAS peptide can have a deletion of amino acid 10 (where the numbering is based on the amino acid sequence set forth in SEQ ID NO:1 or SEQ ID NO:2). In some of these embodiments, the KRAS peptide has a length of 9 amino acids.

As one example, where the KRAS peptide corresponds to amino acids 5-14 (based on the amino acid numbering of a KRAS polypeptide of SEQ ID NO:1 or SEQ ID NO:2), the KRAS peptide can have a deletion of amino acid 7. In some cases, the KRAS peptide has the amino acid sequence KLVVGAGGV (SEQ ID NO:4; KRAS 5, 6, 8-14). In some cases, where the KRAS peptide corresponds to amino acids 5-14 (based on the amino acid numbering of a KRAS polypeptide of SEQ ID NO:1 or SEQ ID NO:2), the KRAS peptide has: i) a deletion of amino acid 7; and ii) one or two amino acid substitutions compared to the wild-type KRAS amino acid sequence. For example, in some cases, the KRAS peptide has the amino acid sequence KLVVGAXGV (SEQ ID NO:5; KRAS 5, 6, 8-14), where X (corresponding to G12 of wild-type KRAS) is an amino acid other than Gly, e.g., where X is Cys, Val, Ser, Ala, Arg, Phe, or Asp. For example, in some cases, the KRAS peptide has the amino acid sequence KLVVGADGV (SEQ ID NO:6; KRAS 5, 6, 8-14 (G12D)). As another example, in some cases, the KRAS peptide has the amino acid sequence KLVVGAVGV (SEQ ID NO:7; KRAS 5, 6, 8-14 (G12V)). As another example, in some cases, the KRAS peptide has the amino acid sequence KLVVGAGXV (SEQ ID NO:8; KRAS 5, 6, 8-14), where X (corresponding to G13 of wild-type KRAS) is an amino acid other than Gly, e.g., where X is Cys, Asp, Arg, Val, Ser, or Ala. In any of the above embodiments, the KRAS peptide can have a length of 9 amino acids.

As another example, where the KRAS peptide corresponds to amino acids 5-14 (based on the amino acid numbering of a KRAS polypeptide of SEQ ID NO:1 or SEQ ID NO:2), the KRAS peptide can have a deletion of amino acid 10. In some cases, the KRAS peptide has the amino acid sequence KLVVVAGGV (SEQ ID NO:9; KRAS 5-9, 11-14). In some cases, where the KRAS peptide corresponds to amino acids 5-14 (based on the amino acid numbering of a KRAS polypeptide of SEQ ID NO:1 or SEQ ID NO:2), the KRAS peptide has: i) a deletion of amino acid 10; and ii) one or two amino acid substitutions compared to the wild-type KRAS amino acid sequence.

For example, in some cases, the KRAS peptide has the amino acid sequence KLVVVAXGV (SEQ ID NO: 10; KRAS 5-9, 11-14), where X (corresponding to G12 of wild-type KRAS) is an amino acid other than Gly, e.g., where X is Cys, Val, Ser, Ala, Arg, Phe, or Asp. For example, in some cases, the KRAS peptide has the amino acid sequence KLVVVADGV (SEQ ID NO: 11; KRAS 5-9, 11-14 (G12D)). As another example, in some cases, the KRAS peptide has the amino acid sequence KLVVVAVGV (SEQ ID NO: 12; KRAS 5-9, 11-14 (G12V)). As another example, in some cases, the KRAS peptide has the amino acid sequence KLVVVAGXV (SEQ ID NO: 13; KRAS 5-9, 11-14), where X (corresponding to G13 of wild-type KRAS) is an amino acid other than Gly, e.g., where X is Cys, Asp, Arg, Val, Ser, or Ala. In any of the above embodiments, the KRAS peptide can have a length of 9 amino acids.

A KRAS peptide present in a pMHC polypeptide is a peptide specifically bound by a T-cell, i.e., the epitope is specifically bound by an epitope-specific T cell, i.e., a T cell having a TCR that is specific for the KRAS epitope. An epitope-specific T cell binds an epitope having a reference amino acid sequence, but does not substantially bind an epitope that differs from the reference amino acid sequence. For example, an epitope-specific T cell binds an epitope having a reference amino acid sequence, and binds an epitope that differs from the reference amino acid sequence, if at all, with an affinity that is less than 10⁻⁶ M, less than 10⁻⁵ M, or less than 10⁻⁴ M. An epitope-specific T cell can bind an epitope for which it is specific with an affinity of at least 10⁻⁷ M, at least 10⁻⁸ M, at least 10⁻⁹ M, or at least 10⁻¹⁰ M.

A KRAS peptide present in a TMP can have a length of at least 4 amino acids, e.g., from 4-25 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa, 20 aa, 21 aa, 22 aa, 23 aa, 24 aa, or 25 aa), including a range of from 6-15 aa, 8-12 aa, 8-16 aa, 8-10 aa, 9-11 aa, 5-10 aa, 10-15 aa, and 15-20 aa in length. In some cases, the peptide is 8, 9, 10, or 11 amino acids in length. In some cases, the KRAS peptide has a length of from 8 amino acids to 12 amino acids.

In some cases, the KRAS peptide present in a TMP presents an epitope specific to an HLA-A, -B, -C, -E, -F, or -G allele. In an embodiment, the epitope peptide present in a pMHC presents an epitope restricted to HLA-A*0101, A*0201, A*0301, A*1101, A*2301, A*2402, A*2407, A*3303, and/or A*3401. In an embodiment, the epitope peptide present in a pMHC polypeptide presents an epitope restricted to HLA-B*0702, B*0801, B*1502, B*3802, B*4001, B*4601, and/or B*5301. In an embodiment, the epitope peptide present in a pMHC polypeptide presents an epitope restricted to C*0102, C*0303, C*0304, C*0401, C*0602, C*0701, C*702, C*0801, and/or C*1502.

In some cases, a suitable KRAS peptide is a peptide of at least 4 amino acids in length, e.g., from 4 amino acids to about 20 amino acids (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa and 20 aa, including a range of from 9-11 amino acids, from 6 to 15 amino acids, from 8 to 12 amino acids, from 8 to 16 amino acids, from 5 to 10 amino acids, from 10 to 15 amino acids, and from 15 to 20 amino acids in length) of a KRAS polypeptide comprising an amino acid sequence having at least 80%, at least 85%, at least 90%, at least 95%, at least 97%, at least 98%, or at least 99%, amino acid sequence identity to one of the following KRAS amino acid sequences:
(A) MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:385), where the KRAS polypeptide comprises one or more (e.g., 1, 2, 3, 4, or 5) amino acid substitutions compared to that sequence (i.e., MTEYKLVVVG AGGVGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:386)), and where the one or more amino acid substitutions can include substitutions associated with cancer; e.g., substitutions that are found in a KRAS polypeptide in a cancer cell;
(B) MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:387), where the KRAS polypeptide comprises one or more (e.g., 1, 2, 3, 4, or 5) amino acid substitutions (i.e., compared to the sequence MTEYKLVVVG AGGVGKSALT IQLIQNHFVD EYDPTIEDSY RKQVVIDGET CLLDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ RVEDAFYTLV REIRQYRLKK ISKEEKTPGC VKIKKCIIM (SEQ ID NO:388)), and where the one or more amino acid substitutions can include substitutions associated with cancer; e.g., substitutions that are found in a KRAS polypeptide in a cancer cell; and
(C) MTEY(X1)L(X2)(X3)(X4)GA(X5)(X6)VGKSALT IQLIQNHFVD EYDPTIEDSY RKQVVIDGET CLWDILDTAG QEEYSAMRDQ YMRTGEGFLC VFAINNTKSF EDIHHYREQI KRVKDSEDVP MVLVGNKCDL PSRTVDTKQA QDLARSYGIP FIETSAKTRQ GVDDAFYTLV REIRKHKEKM SKDGKKKKKK SKTKCVIM (SEQ ID NO:389), where X1 is Lys, Phe, or Leu; X2 is Val or Leu; X3 is Val or Thr; X4 is Val or Thr; X5 is Gly, Asp, Cys, Val, or Ser; and X6 is Gly, Cys, or Asp; where one or both of X5 and X6 is not a Cys.

Non-limiting examples of suitable KRAS peptides include peptides comprising a sequence selected from the group consisting of VVGADGVGK (SEQ ID NO:390), VVGACGVGK (SEQ ID NO:391), VVGAVGVGK (SEQ ID NO:392), VVVGADGVGK (SEQ ID NO:393), VVVGAVGVGK (SEQ ID NO:394), VVVGACGVGK (SEQ ID NO:395), VTGADGVGK (SEQ ID NO:396), VTGAVGVGK (SEQ ID NO:397), VTGACGVGK (SEQ ID NO:398), VTVGADGVGK (SEQ ID NO:399), VTVGAVGVGK (SEQ ID NO:400), and VTVGACGVGK (SEQ ID NO:401); where the KRAS peptide has a length of 9 amino acids or 10 amino acids, or a length of at least 9 amino acids or at least 10 amino acids.

Additional non-limiting examples of suitable KRAS peptides include peptides comprising a sequence selected from the group consisting of: VVVGAGDVGK (SEQ ID NO:402); VVGAGDVGK (SEQ ID NO:403); VVVGARGVGK (SEQ ID NO:404); and VVGARGVGK (SEQ ID NO:405); where the KRAS peptide has a length of 9 amino acids or 10 amino acids, or a length of at least 9 amino acids or at least 10 amino acids.

Non-limiting examples of suitable KRAS peptides include peptides comprising a sequence selected from the group consisting of LVVVGADGV (SEQ ID NO:406), LVVVGAVGV (SEQ ID NO:407), LVVVGACGV (SEQ ID NO:408), KLVVVGADGV (SEQ ID NO:409), KLVVVGAVGV (SEQ ID NO:410), KLVVVGACGV (SEQ ID NO:411), LLVVGADGV (SEQ ID NO:412), LLVVGAVGV (SEQ ID NO:413), LLVVGACGV (SEQ ID NO:414), FLVVVGADGV (SEQ ID NO:415), FLVVVGAVGV (SEQ ID NO:416), and FLVVVGACGV (SEQ ID NO:417); where the KRAS peptide has a length of 9 amino acids or 10 amino acids, or a length of at least 9 amino acids or at least 10 amino acids.

Additional non-limiting examples of suitable KRAS peptides include peptides comprising a sequence selected from the group consisting of: KLVVVGAGDV (SEQ ID NO:418); and KLVVVGARGV (SEQ ID NO:419); where the KRAS peptide has a length of 9 amino acids or 10 amino acids, or a length of at least 9 amino acids or at least 10 amino acids.

Additional non-limiting examples of suitable KRAS peptides include peptides comprising a sequence selected from the group consisting of: GAGDVGKSAL (SEQ ID NO:420); AGDVGKSAL (SEQ ID NO:421); DVGKSALTI (SEQ ID NO:422); GAVGVGKSAL (SEQ ID NO:423); AVGVGKSAL (SEQ ID NO:424); YKLVVVGAV (SEQ ID NO:425); ARGVGKSAL (SEQ ID NO:426); GARGVGKSAL (SEQ ID NO:427); EYKLVVVGAR (SEQ ID NO:428); RGVGKSALTI (SEQ ID NO:429); LVVVGARGV (SEQ ID NO:430); GADGVGKSAL (SEQ ID NO:431); ACGVGKSAL (SEQ ID NO:432); and GACGVGKSAL (SEQ ID NO:433); where the KRAS peptide has a length of 9 amino acids or 10 amino acids, or a length of at least 9 amino acids or at least 10 amino acids.

Additional non-limiting examples of suitable KRAS peptides include peptides comprising a sequence selected from the group consisting of VVVGAGGVGK (SEQ ID NO:434); VVVGACGVGK (SEQ ID NO:435); VVVGADGVGK (SEQ ID NO:436); VVVGARGVGK (SEQ ID NO:437); VVVGAVGVGK (SEQ ID NO:438); and VVGAVGVGK (SEQ ID NO:439), where the KRAS peptide has a length of 9 amino acids or 10 amino acids, or a length of at least 9 amino acids or at least 10 amino acids. Such peptides may present an epitope when bound to an HLA complex comprising a β2M polypeptide and an A*03:01 HLA-A heavy chain. Such peptides may present an epitope when bound to an HLA complex comprising a β2M polypeptide and an HLA A*11:01 heavy chain. In some cases, a peptide such as VVVGARGVGK (SEQ ID NO:440), VVGARGVGK (SEQ ID NO:441), VVVGAVGVGK (SEQ ID NO:442), or VVGAVGVGK (SEQ ID NO:443) and having a length of 9 or 10 amino acids will present an epitope when bound to an HLA complex comprising a β2M polypeptide and an A*30:01 HLA-A heavy chain. In some cases, a peptide such as VVVGACGVGK (SEQ ID NO:444), VVVGADGVGK (SEQ ID NO:445), VVVGARGVGK (SEQ ID NO:446), VVVGAVGVGK (SEQ ID NO:447), or VVGAVGVGK (SEQ ID NO:448), and having a length of 9 or 10 amino acids will present an epitope when bound to an HLA complex comprising a β2M polypeptide and an A*68:01 HLA heavy chain.

Additional non-limiting examples of suitable KRAS peptides include peptides comprising the amino acid sequence GADGVGKSAL (SEQ ID NO:449) or GARGVGKSAL (SEQ ID NO:450), where the KRAS peptide has a length of 9 amino acids or 10 amino acids, or a length of at least 9 amino acids or at least 10 amino acids. Such peptides may present an epitope when bound to an HLA complex comprising a β2M polypeptide and a B*07:02 HLA-B heavy chain.

Additional non-limiting examples of suitable KRAS peptides include peptides comprising a sequence selected from the group consisting of AVGVGKSAL (SEQ ID NO:451), GAVGVGKSAL (SEQ ID NO:452), GAVGVGKSA (SEQ ID NO:453), GACGVGKSA (SEQ ID NO:454), GADGVGKSAL (SEQ ID NO:455), and GADGVGKSA (SEQ ID NO:456), where the KRAS peptide has a length of 9 amino acids or 10 amino acids, or a length of at least 9 amino acids or at least 10 amino acids. In some cases, a peptide such as AVGVGKSAL (SEQ ID NO:457) and having a length of 9 amino acids will present an epitope when bound to an HLA complex comprising a β2M polypeptide and a C*01:02 HLA-C heavy chain. In some cases, a peptide such as GAVGVGKSAL (SEQ ID NO:458) and having a length of 10 amino acids, or a peptide such as GAVGVGKSA (SEQ ID NO:459) and having a length of 9 amino acids, will present an epitope when bound to an HLA complex comprising a β2M polypeptide and a C*03:03 HLA-C heavy chain. In some cases, a peptide such as GACGVGKSA (SEQ ID NO:460), GADGVGKSAL (SEQ ID NO:461), GAVGVGKSAL (SEQ ID NO:462), or GAVGVGKSA (SEQ ID NO:463). and having a length of 9 amino acids or 10 amino acids, will present an epitope when bound to an HLA complex comprising a β2M polypeptide and a C*03:04 HLA-C heavy chain. In some cases, a peptide such as GADGVGKSAL (SEQ ID NO:464) and having a length of 10 amino acids, or a peptide such as GADGVGKSA (SEQ ID NO:465) and having a length of 9 amino acids, will present an epitope when bound to an HLA complex comprising a β2M polypeptide and a C*08:02 HLA-C heavy chain.

In some cases, a pMHC polypeptide modulates the activity of a T cell that comprises a TCR that is specific for a G12V form of a KRAS polypeptide, as described above. In such cases, the KRAS peptide present in a pMHC polypeptide can comprise, e.g., one of the following amino acid sequences: VVGAVGVGK (SEQ ID NO:466), VVVGAVGVGK (SEQ ID NO:467), VGAVGVGKS (SEQ ID NO:468), VGAVGVGKSA (SEQ ID NO:469), AVGVGKSAL (SEQ ID NO:470), AVGVGKSALT (SEQ ID NO:471), GAVGVGKSAL (SEQ ID NO:472), GAVGVGKSA (SEQ ID NO:473), LVVVGAVGVG (SEQ ID NO:474), LVVVGAVGV (SEQ ID NO:475), KLVVVGAVGV (SEQ ID NO:476), and KLVVVGAVG (SEQ ID NO:477); where the KRAS peptide has a length of 9 amino acids or 10 amino acids, or a length of at least 9 amino acids or at least 10 amino acids.

In some cases, the KRAS peptide present in a pMHC polypeptide presents an epitope specific to an HLA-A, -B, -C, -E, -F, or -G allele. In an embodiment, the KRAS peptide present in a pMHC polypeptide presents an epitope restricted to HLA-A*0101, A*0201, A*0203, A*0301, A*1101, A*2301, A*2402, A*2407, A*3101, A*3303, A*3401, and/or A*6801. In an embodiment, the KRAS epitope peptide present in a pMHC polypeptide presents an epitope restricted to HLA-B*0702, B*0801, B*1502, B*2705, B*3802, B*3802, B*3901, B*3902, B*4001, B*4601, B*5101, and/or B*5301. In an embodiment, the KRAS epitope peptide present in a pMHC polypeptide presents an epitope restricted to C*0102, C*0303, C*0304, C*0401, C*0602, C*0701, C*702, C*0801, and/or C*1502.

As non-limiting examples, KRAS peptides comprising a peptide selected from the group consisting of VVGADGVGK (SEQ ID NO:478), VVGACGVGK (SEQ ID NO:479), VVGAVGVGK (SEQ ID NO:480), VVVGADGVGK (SEQ ID NO:481), VVVGAVGVGK (SEQ ID NO:482), VVVGACGVGK (SEQ ID NO:483), VTGADGVGK (SEQ ID NO:484), VTGAVGVGK (SEQ ID NO:485), VTGACGVGK (SEQ ID NO:486), VTVGADGVGK (SEQ ID NO:487), VTVGAVGVGK (SEQ ID NO:488), VTVGACGVGK (SEQ ID NO:489), VVVGAGDVGK (SEQ ID NO:490), VVGAGDVGK (SEQ ID NO:491), VVVGARGVGK (SEQ ID NO:492), and VVGARGVGK (SEQ ID NO:493), where the KRAS peptide has a length of 9 amino acids or 10 amino acids, or a length of at least 9 amino acids or at least 10 amino acids, present an epitope when bound to an HLA complex comprising a β2M polypeptide and an A*1101 HLA-A heavy chain. Such peptides may also be presented in complex with an HLA complex comprising a β2M polypeptide and an A*6801 HLA-A heavy chain.

As non-limiting examples, KRAS peptides comprising a peptide selected from the group consisting of LVVVGADGV (SEQ ID NO:494), LVVVGAVGV (SEQ ID NO:495), LVVVGACGV (SEQ ID NO:496), KLVVVGADGV (SEQ ID NO:497), KLVVVGAVGV (SEQ ID NO:498), KLVVVGACGV (SEQ ID NO:499), LLVVGADGV (SEQ ID NO:500), LLVVGAVGV (SEQ ID NO:501), LLVVGACGV (SEQ ID NO:502), FLVVVGADGV (SEQ ID NO:503), FLVVVGAVGV (SEQ ID NO:504), and FLVVVGACGV (SEQ ID NO:505) where the KRAS peptide has a length of 9 amino acids or 10 amino acids, or a length of at least 9 amino acids or at least 10 amino acids, present an epitope when bound to an HLA complex comprising a β2M polypeptide and an A*0201 HLA-A heavy chain.

As additional examples, the following KRAS peptides can present an epitope when bound to an HLA complex comprising a β2M polypeptide and an HLA-A heavy chain as follows: GAGDVGKSAL (SEQ ID NO:506), which can present an epitope when bound to an HLA complex comprising a β2M polypeptide and a B*3801 HLA-A heavy chain; AGDVGKSAL (SEQ ID NO:507), which can present an epitope when bound to an HLA complex comprising a β2M polypeptide and a B0702, a B*3801, or a B*3901 HLA-A heavy chain; DVGKSALTI (SEQ ID NO:508), which can present an epitope when bound to an HLA complex comprising a β2M polypeptide and a B*5101 HLA-A heavy chain; GAVGVGKSAL (SEQ ID NO:509), which can present an epitope when bound to an HLA complex comprising a β2M polypeptide and a B*0702 or a B*3801 HLA-A heavy chain; AVGVGKSAL (SEQ ID NO:510), which can present an epitope when bound to an HLA complex comprising a β2M polypeptide and a B*0702 HLA-A heavy chain; YKLVVVGAV (SEQ ID NO:511), which can present an epitope when bound to an HLA complex comprising a β2M polypeptide and an A*0203 or a B*3902 HLA-A heavy chain; ARGVGKSAL (SEQ ID NO:512), which can present an epitope when bound to an HLA complex comprising a β2M polypeptide and a B*0702, a B*2705, or a B*3901 HLA-A heavy chain; GARGVGKSAL (SEQ ID NO:513), which can present an epitope when bound to an HLA complex comprising a β2M polypeptide and a B*0702 HLA-A heavy chain; EYKLVVVGAR (SEQ ID NO:514), which can present an epitope when bound to an HLA complex comprising a β2M polypeptide and an A*3101 HLA-A heavy chain; RGVGKSALTI (SEQ ID NO:515), which can present an epitope when bound to an HLA complex comprising a β2M polypeptide and a B*0702 HLA-A heavy chain; LVVVGARGV (SEQ ID NO:516), which can present an epitope when bound to an HLA complex comprising a β2M polypeptide and an A*0203 HLA-A heavy chain; GADGVGKSAL (SEQ ID NO:517), which can present an epitope when bound to an HLA complex comprising a β2M polypeptide and a B*3801 HLA-A heavy chain; ACGVGKSAL (SEQ ID NO:518), which can present an epitope when bound to an HLA complex comprising a β2M polypeptide and a B*0702 HLA-A heavy chain; and GACGVGKSAL (SEQ ID NO:519), which can present an epitope when bound to an HLA complex comprising a β2M polypeptide and a B*3801 HLA-A heavy chain.

Whether a given peptide (e.g., a KRAS peptide that comprises a KRAS epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays, ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Second Peptide Linker

The second peptide linker present in a pMHC polypeptide can have a length of from about 5 amino acids to about 50 amino acids, e.g., from about 5 amino acids to about 10 amino acids, from about 10 amino acids to about 15 amino acids, from about 15 amino acids to about 20 amino acids, from about 20 amino acids to about 25 amino acids, from about 25 amino acids to about 30 amino acids, from about 30 amino acids to about 35 amino acids, from about 35 amino acids to about 40 amino acids, from about 40 amino acids to about 45 amino acids, or from about 45 amino acids to about 50 amino acids.

The second peptide linker can be a flexible peptide linker or a rigid peptide linker.

The second peptide linker can comprise a glycine polymer (G)ₙ, a glycine-serine polymer (including, for example, (GS)ₙ, (GSGGS)ₙ (SEQ ID NO:325), (GGGGS)n (SEQ ID NO:326), and (GGGS)˜(SEQ ID NO:327), where n is an integer of at least one and can be an integer from 1 to 10.

Exemplary flexible peptide linkers include, e.g., (GGGGS)n (SEQ ID NO:328); also referred to as a “G4S” linker), where n is 1, 2, 3, 4, 5, 6, 7, 8, 9, or 10. In some cases, a linker comprises the amino acid sequence (GGGGS)n (SEQ ID NO:328), where n is 1, 2, 3, 4, 5, 6, 7, 8, 9 or 10. In some cases, a linker comprises the amino acid sequence (GGGGS)n (SEQ ID NO:329), where n is 2. In some cases, a linker comprises the amino acid sequence (GGGGS)n (SEQ ID NO:330), where n is 3. In some cases, a linker comprises the amino acid sequence (GGGGS)n (SEQ ID NO:331), where n is 4. In some cases, a linker comprises the amino acid sequence (GGGGS)n (SEQ ID NO:332), where n is 7. In some cases, a linker comprises the amino acid sequence AAAGG (SEQ ID NO:333). Also suitable is a linker having the amino acid sequence AAAGG (SEQ ID NO:333).

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:356), A(EAAAK)n (SEQ ID NO:357), A(EAAAK)nA (SEQ ID NO:358), A(EAAAK)nALEA(EAAAK)nA (SEQ ID NO:359), (Lys-Pro)n (SEQ ID NO:360), (Glu-Pro)n (SEQ ID NO:361), (Thr-Pro-Arg)n (SEQ ID NO:362), and (Ala-Pro)n (SEQ ID NO:363) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:364) include EAAAK (SEQ ID NO:364), (EAAAK)₂ (SEQ ID NO:365), (EAAAK)₃ (SEQ ID NO:366), A(EAAAK)₄ALEA(EAAAK)₄A (SEQ ID NO:376), and AEAAAKEAAAKA (SEQ ID NO:368). Non-limiting examples of suitable rigid peptide linkers comprising (AP)n include PAPAP (SEQ ID NO:369; also referred to herein as “(AP)2”); APAPAPAP (SEQ ID NO:370; also referred to herein as “(AP)4”); APAPAPAPAPAP (SEQ ID NO:371; also referred to herein as “(AP)6”); APAPAPAPAPAPAPAP (SEQ ID NO:372; also referred to herein as “(AP)8”); and APAPAPAPAPAPAPAPAPAP (SEQ ID NO:373; also referred to herein as “(AP)10”). Non-limiting examples of suitable rigid peptide linkers comprising (KP)n include KPKP (SEQ ID NO:374; also referred to herein as “(KP)2”); KPKPKPKP (SEQ ID NO:375; also referred to herein as “(KP)4”); KPKPKPKPKPKP (SEQ ID NO:376; also referred to herein as “(KP)6”); KPKPKPKPKPKPKPKP (SEQ ID NO:377; also referred to herein as “(KP)8”); and KPKPKPKPKPKPKPKPKPKP (SEQ ID NO:378; also referred to herein as “(KP)10”). Non-limiting examples of suitable rigid peptide linkers comprising (EP)n include EPEP (SEQ ID NO:379; also referred to herein as “(EP)2”); EPEPEPEP (SEQ ID NO:380; also referred to herein as “(EP)4”); EPEPEPEPEPEP (SEQ ID NO:381; also referred to herein as “(EP)6”); EPEPEPEPEPEPEPEP (SEQ ID NO:382; also referred to herein as “(EP)8”); and EPEPEPEPEPEPEPEPEPEP (SEQ ID NO:383; also referred to herein as “(EP)10”).

### Immunomodulatory Polypeptides (MODs)

In some cases, a MOD present in a TMP is a wild-type (“wt”) MOD. As discussed above, in other cases, a MOD present in a TMP is a variant of a wt. MOD that has reduced affinity for a co-MOD compared to the affinity of a corresponding wild-type MOD for the co-MOD. Suitable MODs that exhibit reduced affinity for a co-MOD can have from 1 amino acid (aa) to 20 aa differences from a wild-type MOD. For example, in some cases, a variant MOD present in a TMP differs in amino acid sequence by 1 aa, 2 aa, 3 aa, 4 aa, 5 aa, 6 aa, 7 aa, 8 aa, 9 aa, or 10 aa, from a corresponding wild-type MOD. As another example, in some cases, a variant MOD present in a TMP differs in amino acid sequence by 11 aa, 12 aa, 13 aa, 14 aa, 15 aa, 16 aa, 17 aa, 18 aa, 19 aa, or 20 aa, from a corresponding wild-type MOD.

As discussed above, a MOD may comprise a variant of a wt MOD that may exhibit reduced binding to its co-MOD, including e.g., reduced binding to one or more chains or domains of the co-MOD. For example, a variant MOD present in a TMP may bind its co-MOD with an affinity that it at least 1000 less, at least 15% less, at least 20% less, at least 25% less, at least 30% less, at least 35% less, at least 40% less, at least 45% less, at least 500% less, at least 5500 less, at least 60% less, at least 65% less, at least 70% less, at least 75% less, at least 80% less, at least 85% less, at least 90% less, at least 95% less, or more than 95% less, than the affinity of a corresponding wild-type MOD for the co-MOD.

Exemplary pairs of MODs and their co-MODs include, but are not limited to those set out in Table 1, below:

TABLE 1

| Immunomodulatory | |
|---|---|
| Polypeptide (MOD) | Co-MOD |
| 4-1BBL | 4-1BB |
| PD-L1 | PD-1 |
| IL-2 | IL-2 receptor |
| CD80 | CD28 |
| CD86 | CD28 |
| OX40L (CD252) | OX40 (CD134) |
| Fas ligand | Fas |
| ICOS-L | ICOS |
| ICAM | LFA-1 |
| CD30L | CD30 |
| CD40 | CD40L |
| CD83 | CD83L |
| HVEM (CD270) | CD160 |
| JAG1 (CD339) | Notch |
| JAG1 | CD46 |
| CD80 | CTLA4 |
| CD86 | CTLA4 |
| CD70 | CD27 |
| TGFβ | TGFβ receptor |

As depicted schematically in FIG. 14, one or more MODs can be present in a TMP at any of a variety of positions. FIG. 14 depicts the position of two copies of a variant IL-2 polypeptide; however, the MOD can be any number of and any of a variety of MODs, as described herein. As depicted in FIG. 14, a MOD can be: 1) C-terminal to the MHC class I heavy chain and N-terminal to the Ig Fc polypeptide; in other words, between the MHC class I heavy chain polypeptide and the Ig Fc polypeptide, which is referred to as “Position 2” in FIG. 14; 2) C-terminal to the Ig Fc polypeptide, which is referred to as “Position 3” in FIG. 14; or 3) N-terminal to the peptide epitope, which is referred to as “Position 4” in FIG. 14.

Wild-type immunomodulatory polypeptides and variants, including reduced affinity variants, such as PD-L1, CD80, CD86, 4-1BBL and IL-2 are described in the published literature, e.g., published PCT applications WO2020132138A1 and WO2019/051091, the disclosures of which as they pertain to MODs and specific variant MODs of PD-L1, CD80, CD86, 4-1BBL, IL-2 are expressly incorporated herein by reference, including specifically paragraphs [00260]-[00455] of WO2020132138A1 and paragraphs [00157]-[00352] of WO2019/051091.

Of specific interest are MODs that are variants of the cytokine IL-2. Wild-type IL-2 binds to IL-2 receptor (IL-2R) on the surface of a T cell. Wild-type IL-2 has a strong affinity for IL-2R and will bind to activate most or substantially all CD8+ T cells. For this reason, synthetic forms of wild type IL-2 such as the drug Aldesleukin (trade name Proleukin®) are known to have severe side-effects when administered to humans for the treatment of cancer because the IL-2 indiscriminately activates both target and non-target T cells.

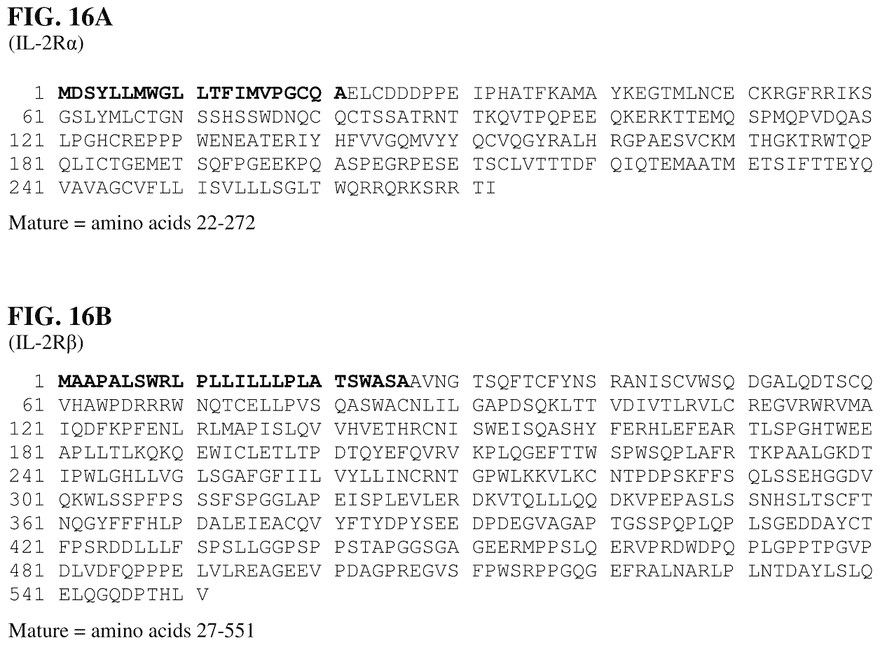

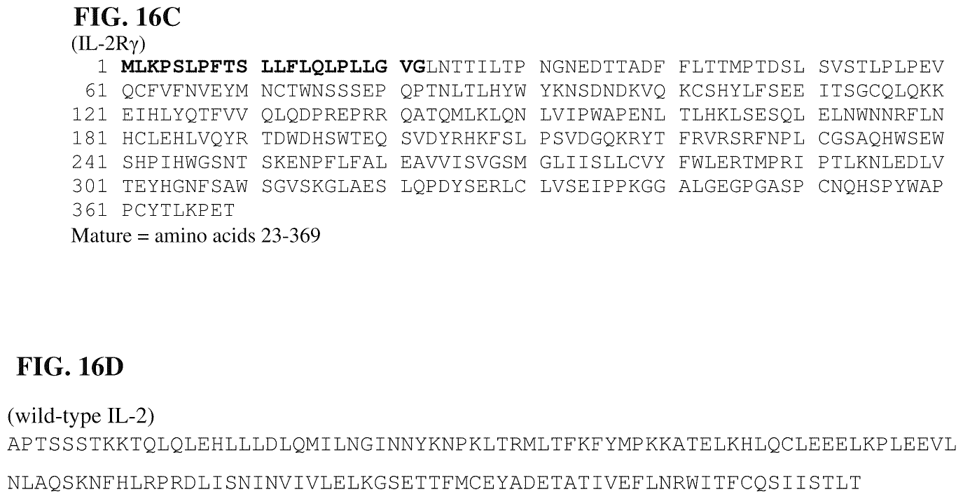

An IL-2 receptor is in some cases a heterotrimeric polypeptide comprising an alpha chain (IL-2Rα; also referred to as CD25), a beta chain (IL-2Rβ; also referred to as CD122; and a gamma chain (IL-2Rγ; also referred to as CD132). Amino acid sequences of human IL-2, human IL-2Rα, IL2Rβ, and IL-2Rγ are known. See, e.g., published PCT applications WO2020132138A1 and WO2019/051091, discussed above. For example, a wild-type IL-2 polypeptide can have the amino acid sequence depicted in FIG. 16D. Amino acid sequences of human IL-2Rα, human IL-2Rβ, and human IL-2Rγ are depicted in FIGS. 16A, 16B, and 16C, respectively, where the mature form of IL-2Rα is amino acids 22-272 of the amino acid sequence depicted in FIG. 16A, the mature form of IL-2Rβ is amino acids 27-551 of the amino acid sequence depicted in FIG. 16B, and the mature form of IL-2Rγ is amino acids 23-369 of the amino acid sequence depicted in FIG. 16C.

In some cases, an IL-2 variant MOD of this disclosure exhibits decreased binding to IL-2Rα, thereby minimizing or substantially reducing the activation of Tregs by the IL-2 variant. Alternatively, or additionally, in some cases, an IL-2 variant MOD of this disclosure exhibits decreased binding to IL-2Rβ and/or IL-2Rγ such that the IL-2 variant MOD exhibits an overall reduced affinity for IL-2R. In some cases, an IL-2 variant MOD of this disclosure exhibits both properties, i.e., it exhibits decreased or substantially no binding to IL-2Rα, and also exhibits decreased binding to IL-2Rβ and/or IL-2Rγ such that the IL-2 variant polypeptide exhibits an overall reduced affinity for IL-2β. For example, IL-2 variants having substitutions at H16 and F42 have shown decreased binding to IL-2Rα and IL-2Rβ. See, Quayle et al., Clin Cancer Res; 26(8) Apr. 15, 2020, which discloses that the binding affinity of an IL-2 polypeptide with H16A and F42A substitutions for human IL-2Rα and IL-2Rβ was decreased 110- and 3-fold, respectively, compared with wild-type IL2 binding, predominantly due to a faster off-rate for each of these interactions. TMPs comprising such variants, including variants that exhibit decreased binding to IL-2Rα and IL-2Rβ, have shown the ability to preferentially bind to and activate IL-2 receptors on T cells that contain the target TCR that is specific for the peptide epitope on the TMP, and are thus less likely to deliver IL-2 to non-target T cells, i.e., T cells that do not contain a TCR that specifically binds the peptide epitope on the TMP. That is, the binding of the IL-2 variant MOD to its costimulatory polypeptide on the T cell is substantially driven by the binding of the MHC-epitope moiety rather than by the binding of the IL-2.

Suitable IL-2 variant MODs thus include a polypeptide that comprises an amino acid sequence having at least 90%, at least 95%, at least 97%, at least 98%, or at least 99% amino acid sequence identity to the wild-type IL-2 amino acid sequence depicted in FIG. 16D; and that have one or more amino acid differences from the wild-type IL-2 amino acid sequence depicted in FIG. 16D. In some cases, such a variant IL-2 polypeptide of this disclosure exhibits reduced binding affinity to IL-2R, compared to the binding affinity of an IL-2 polypeptide comprising the wild-type IL-2 amino acid sequence depicted in FIG. 16D. For example, in some cases, a variant IL-2 polypeptide binds IL-2R with a binding affinity that is at least 10% less, at least 15% less, at least 20% less, at least 25%, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, at least 95% less, or more than 95% less, than the binding affinity of an IL-2 polypeptide comprising the wild-type IL-2 amino acid sequence depicted in FIG. 16D for an IL-2R (e.g., an IL-2R comprising polypeptides comprising the amino acid sequences depicted in FIG. 16A-16C, e.g., the mature forms of the amino acid sequences depicted in FIG. 16A-16C), when assayed under the same conditions.

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:334), where X₁ is an amino acid other than His, and where X₂ is an amino acid other than Phe. In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEX₁LLLD LQMILNGINN YKNPKLTRML TX₂KFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:335), where: i) X₁ is Ala, Arg, Asn, Asp, Cys, Gln, Glu, Gly, Ile, Leu, Lys, Met, Phe, Pro, Ser, Thr, Trp, Tyr, or Val; and ii) X₂ is Ala, Arg, Asn, Asp, Cys, Gln, Glu, Gly, His, Ile, Leu, Lys, Met, Pro, Ser, Thr, Trp, Tyr, or Val. In some cases, X₁ is Ala and X₂ is Ala. In some cases, X₁ is Thr and X₂ is Ala. In some cases, X₁ is Asp and X₂ is Ala. In some cases, X₁ is Glu and X₂ is Ala.

In some cases, a suitable variant IL-2 polypeptide comprises an amino acid sequence having at least 90%, at least 95%, at least 97%, at least 98%, at least 99%, or 100% amino acid sequence identity to the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:336), i.e., the variant IL-2 polypeptide has the amino acid sequence of wild-type IL-2 but with H16A and F42A substitutions (shown in bold). Alternatively, the foregoing sequence, but with substitutions other than Ala at H16 and/or F42 may be employed, e.g., H16T may be employed instead of H16A. In some cases, a variant IL-2 polypeptide present in a TMP comprises the amino acid sequence: APTSSSTKKT QLQLEALLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:337). In some cases, a variant IL-2 polypeptide present in a TMP comprises the amino acid sequence: APTSSSTKKT QLQLETLLLD LQMILNGINN YKNPKLTRML TAKFYMPKKA TELKHLQCLEEELKPLEEVL NLAQSKNFHL RPRDLISNIN VIVLELKGSE TTFMCEYADE TATIVEFLNRWITFCQSIIS TLT (SEQ ID NO:338). In some cases, a TMP comprises two copies of such a variant IL-2 polypeptide. Where a TMP comprises two copies of a variant IL-2 polypeptide, in some cases, the two copies are in tandem. Where a TMP comprises two copies of a variant IL-2 polypeptide, and where the two copies are in tandem, in some cases, the TMP comprises a peptide linker between the two copies.

Some exemplary combinations of mutations that reduce binding of an IL-2 variant polypeptide to IL-2Rα and IL-2Rβ include the following from Table 2:

TABLE 2

| Mutation(s) to | Mutation(s) to | |
|---|---|---|
| decrease binding | decrease binding | |
| to IL-2Rα | to IL-2Rβ | Exemplary combinations |
| R38 with any | E15 with any | E15A with R38A, R38D or R38E |
| amino acid other | amino acid other | |
| than Arg, e.g., Ala, | than Glu | |
| Asp, Glu | | |
| R38 with any | H16, with any | H16A with R38A, R38D or R38E |
| amino acid other | amino acid other | H16T with R38A, R38D or R38E |
| than Arg, e.g., Ala, | than His, e.g., Ala, | H16E with R38A, R38D or R38E |
| Asp, Glu | Glu, Thr, or Asp, | H16D with R38A, R38D or R38E |
| R38 with any | D84, with any | D84H with R38A, R38D or R38E |
| amino acid other | amino acid other | D84K with R38A, R38D or R38E |
| than Arg, e.g., Ala, | than Asp, e.g., His, | D84R with R38A, R38D or R38E |
| Asp, Glu | Lys or Arg | |
| R38 with any | N88, with any | R38A with N88S, N88A, N88G, N88R, N88T, or N88D |
| amino acid other | amino acid other | R38D with N88S, N88A, N88G, N88R, N88T, or N88D |
| than Arg, e.g., Ala, | than Asn, e.g., Ser, | R38E with N88S, N88A, N88G, N88R, N88T, or N88D |
| Asp, Glu | Ala, Gly, Arg, Thr | |
| | or Asp | |
| R38 with any | V91 with any | R38A with V91E, V91A or V91T |
| amino acid other | amino acid other | R38D with V91E, V91A or V91T |
| than Arg, e.g., Ala, | than Val, e.g., Glu, | R38E with V91E, V91A or V91T |
| Asp, Glu | Ala or Thr | |
| R38 with any | I92 with any amino | R38A, I92A |
| amino acid other | acid other than Ile, | R38D, I92A |
| than Arg, e.g., Ala, | e.g., Ala | R38E, I92A |
| Asp, Glu | | |
| F42, with any | E15 with any | E15A, F42A |
| amino acid other | amino acid other | E15A, F42K |
| than Phe, e.g., Ala | than Glu | |
| or Lys, , as well as | | |
| Met, Pro, Ser, Thr, | | |
| Trp, Tyr, and Val | | |
| F42, with any | H16, with any | H16A, F42A; H16T, F42A; |
| amino acid other | amino acid other | H16E, F42A; H16D, F42A |
| than Phe, e.g., Ala | than His, e.g., Ala, | H16A, F42K; H16T, F42K; |
| or Lys, as well as | Glu, Thr, or Asp, | H16E, F42K; H16D, F42K |
| Met, Pro, Ser, Thr, | | |
| Trp, Tyr, and Val | | |
| F42, with any | D84, with any | F42A with D84H, D84K or D84R |
| amino acid other | amino acid other | F42K with D84H, D84K or D84R |
| than Phe, e.g., Ala | than Asp, e.g., His, | |
| or Lys, as well as | Lys or Arg | |
| Met, Pro, Ser, Thr, | | |
| Trp, Tyr, and Val | | |
| F42, with any | N88, with any | F42A with N88S, N88A, N88G, N88R, N88T, or N88D |
| amino acid other | amino acid other | F42K with N88S, N88A, N88G, N88R, N88T, or N88D |
| than Phe, e.g., Ala | than Asn, e.g., Ser, | |
| or Lys, as well as | Ala, Gly, Arg, Thr | |
| Met, Pro, Ser, Thr, | or Asp | |
| Trp, Tyr, and Val | | |
| F42, with any | V91 with any | F42A with V91E, V91A, or V91T |
| amino acid other | amino acid other | F42K with V91E, V91A, or V91T |
| than Phe, e.g., Ala | than Val, e.g., Glu, | |
| or Lys, as well as | Ala or Thr | |
| Met, Pro, Ser, Thr, | | |
| Trp, Tyr, and Val | | |
| F42, with any | I92 with any amino | F42A with I92A |
| amino acid other | acid other than Ile, | F42K with I92A |
| than Phe, e.g., Ala | e.g., Ala | |
| or Lys, as well as | | |
| Met, Pro, Ser, Thr, | | |
| Trp, Tyr, and Val | | |
| K43, with any | E15 with any | E15A, K43E |
| amino acid other | amino acid other | |
| than Lys, e.g., Glu | than Glu | |
| K43, with any | H16, with any | H16A, K43E; H16T, K43E; |
| amino acid other | amino acid other | H16E, K43E; H16D, K43E |
| than Lys, e.g., Glu | than His, e.g., Ala, | |
| | Glu, Thr, or Asp, | |
| K43, with any | D84, with any | K43E with D84H, D84K or D84R |
| amino acid other | amino acid other | |
| than Lys, e.g., Glu | than Asp, e.g., His, | |
| | Lys or Arg | |
| K43, with any | N88, with any | K43E with N88S, N88A, N88G, N88R, N88T, or N88D |
| amino acid other | amino acid other | |
| than Lys, e.g., Glu | than Asn, e.g., Ser, | |
| | Ala, Gly, Arg, Thr | |
| | or Asp | |
| K43, with any | V91 with any | K43E with V91E, V91A, or V91T |
| amino acid other | amino acid other | |
| than Lys, e.g., Glu | than Val, e.g., Glu, | |
| | Ala or Thr | |
| K43, with any | I92 with any amino | K43E, I92A |
| amino acid other | acid other than Ile, | |
| than Lys, e.g., Glu | e.g., Ala | |
| E62, with any | E15 with any | E15A, E62Q |
| amino acid other | amino acid other | |
| than Glu, e.g., Gln | than Glu | |
| E62, with any | H16, with any | H16A, E62Q; H16T, E62Q; |
| amino acid other | amino acid other | H16E, E62Q; H16D, E62Q |
| than Glu, e.g., Gln | than His, e.g., Ala, | |
| | Glu, Thr, or Asp, | |
| E62, with any | D84, with any | E62Q with D84H, D84K or D84R |
| amino acid other | amino acid other | |
| than Glu, e.g., Gln | than Asp, e.g., His, | |
| | Lys or Arg | |
| E62, with any | N88, with any | E62Q with N88S, N88A, N88G, N88R, N88T, or N88D |
| amino acid other | amino acid other | |
| than Glu, e.g., Gln | than Asn, e.g., Ser, | |
| | Ala, Gly, Arg, Thr | |
| | or Asp | |
| E62, with any | V91 with any | E62Q with V91E, V91A, or V91T |
| amino acid other | amino acid other | |
| than Glu, e.g., Gln | than Val, e.g., Glu, | |
| | Ala or Thr | |
| E62, with any | I92 with any amino | E62Q, I92A |
| amino acid other | acid other than Ile, | |
| than Glu, e.g., Gln | e.g., Ala | |
| F42, with any | E15 with any | E15A, F42A with D84H, D84K or D84R |
| amino acid other | amino acid other | E15A, F42K with D84H, D84K or D84R |
| than Phe, e.g., Ala | than Glu | |
| or Lys, as well as | D84, with any | |
| Met, Pro, Ser, Thr, | amino acid other | |
| Trp, Tyr, and Val | than Asp, e.g., His, | |
| | Lys and Arg | |
| F42, with any | E15 with any | E15A, F42A with N88S, N88A, N88G, N88R, N88T, or |
| amino acid other | amino acid other | N88D |
| than Phe, e.g., Ala | than Glu | E15A, F42K with N88S, N88A, N88G, N88R, N88T, or |
| or Lys, as well as | N88, with any | N88D |
| Met, Pro, Ser, Thr, | amino acid other | |
| Trp, Tyr, and Val | than Asn, e.g., Ser, | |
| | Ala, Gly, Arg, Thr, | |
| | and Asp | |
| F42, with any | E15 with any | E15A, F42A with V91E, V91A, or V91T |
| amino acid other | amino acid other | E15A, F42K with V91E, V91A, or V91T |
| than Phe, e.g., Ala | than Glu | |
| or Lys, as well as | V91 with any | |
| Met, Pro, Ser, Thr, | amino acid other | |
| Trp, Tyr, and Val | than Val, e.g., Glu, | |
| | Ala or Thr | |
| F42, with any | E15 with any | E15A, F42A with I92A |
| amino acid other | amino acid other | E15A, F42K with I92A |
| than Phe, e.g., Ala | than Glu | |
| or Lys, as well as | I92 with any amino | |
| Met, Pro, Ser, Thr, | acid other than Ile, | |
| Trp, Tyr, and Val | e.g., Ala | |
| F42, with any | H16, with any | H16A, F42A with D84H, D84K or D84R |
| amino acid other | amino acid other | H16A, F42K with D84H, D84K or D84R |
| than Phe, e.g., Ala | than His, e.g., Ala, | H16T, F42A with D84H, D84K or D84R |
| or Lys, as well as | Glu, Thr, or Asp | H16T, F42K with D84H, D84K or D84R |
| Met, Pro, Ser, Thr, | D84, with any | H16E, F42A with D84H, D84K or D84R |
| Trp, Tyr, and Val | amino acid other | H16E, F42K with D84H, D84K or D84R |
| | than Asp, e.g., His, | H16D, F42A with D84H, D84K or D84R |
| | Lys and Arg | H16D, F42K with D84H, D84K or D84R |
| F42, with any | H16, with any | H16A, F42A with N88S, N88A, N88G, N88R, N88T, or |
| amino acid other | amino acid other | N88D |
| than Phe, e.g., Ala | than His, e.g., Ala, | H16A, F42K with N88S, N88A, N88G, N88R, N88T, or |
| or Lys, as well as | Glu, Thr, or Asp | N88D |
| Met, Pro, Ser, Thr, | N88, with any | H16T, F42A with N88S, N88A, N88G, N88R, N88T, or |
| Trp, Tyr, and Val | amino acid other | N88D |
| | than Asn, e.g., Ser, | H16T, F42K with N88S, N88A, N88G, N88R, N88T, or |
| | Ala, Gly, Arg, Thr, | N88D |
| | and Asp | H16E, F42A with N88S, N88A, N88G, N88R, N88T, or |
| | | N88D |
| | | H16E, F42K with N88S, N88A, N88G, N88R, N88T, or |
| | | N88D |
| | | H16D, F42A with N88S, N88A, N88G, N88R, N88T, or |
| | | N88D |
| | | H16D, F42K with N88S, N88A, N88G, N88R, N88T, or |
| | | N88D |
| F42, with any | H16, with any | H16A, F42A with V91E, V91A, or V91T |
| amino acid other | amino acid other | H16A, F42K with V91E, V91A, or V91T |
| than Phe, e.g., Ala | than His, e.g., Ala, | H16T, F42A with V91E, V91A, or V91T |
| or Lys, as well as | Glu, Thr, or Asp | H16T, F42K with V91E, V91A, or V91T |
| Met, Pro, Ser, Thr, | V91 with any | H16E, F42A with V91E, V91A, or V91T |
| Trp, Tyr, and Val | amino acid other | H16E, F42K with V91E, V91A, or V91T |
| | than Val, e.g., Glu, | H16D, F42A with V91E, V91A, or V91T |
| | Ala or Thr | H16D, F42K with V91E, V91A, or V91T |
| F42, with any | H16, with any | H16A, F42A with I92A |
| amino acid other | amino acid other | H16A, F42K with I92A |
| than Phe, e.g., Ala | than His, e.g., Ala, | H16T, F42A with I92A |
| or Lys, as well as | Glu, Thr, or Asp | H16T, F42K with I92A |
| Met, Pro, Ser, Thr, | I92 with any amino | H16E, F42A with I92A |
| Trp, Tyr, and Val | acid other than Ile, | H16E, F42K with I92A |
| | e.g., Ala | H16D, F42A with I92A |
| | | H16D, F42K with I92A |

In some cases, a MOD present in a TMP is a PD-L1 polypeptide. In some cases, a PD-L1 polypeptide of a TMP comprises an amino acid sequence having at least 75%, at least 80%, at least 85%, at least 90%, at least 95%, at least 97%, at least 98%, at least 99%, or 100%, amino acid sequence identity to the following PD-L1 ectodomain amino acid sequence: FT VTVPKDLYVV EYGSNMTIEC KFPVEKQLDL AALIVYWEME DKNIIQFVHG EEDLKVQHSS YRQRARLLKD QLSLGNAALQ ITDVKLQDAG VYRCMISYGG ADYKRITVKV NAPYNKINQR ILVVDPVTSE HELTCQAEGY PKAEVIWTSS DHQVLSGKTT TTNSKREEKL FNVTSTLRIN TTTNEIFYCT FRRLDPEENH TAELVIPGNI LNVSIKI (SEQ ID NO:341).

In some cases, a MOD present in a TMP is a 4-1BBL polypeptide. In some cases, a 4-1BBL polypeptide of a TMP comprises an amino acid sequence having at least 75%, at least 80%, at least 85%, at least 90%, at least 95%, at least 97%, at least 98%, at least 99%, or 100%, amino acid sequence identity to the following 4-1BBL amino acid sequence: DPAGLLDLRQG MFAQLVAQNV LLIDGPLSWY SDPGLAGVSL TGGLSYKEDT KELVVAKAGV YYVFFQLELR RVVAGEGSGS VSLALHLQPL RSAAGAAALA LTVDLPPASS EARNSAFGFQ GRLLHLSAGQ RLGVHLHTEA RARHAWQLTQ GATVLGLFRV TPEIPA (SEQ ID NO:342).

In some cases, a MOD present in a TMP is an ICOS-L polypeptide. In some cases, an ICOS-L polypeptide of a TMP comprises an amino acid sequence having at least 75%, at least 80%, at least 85%, at least 90%, at least 95%, at least 97%, at least 98%, at least 99%, or 100%, amino acid sequence identity to the following ICOS-L amino acid sequence: QEKEVRAMVG SDVELSCACP EGSRFDLNDV YVYWQTSESK TVVTYHIPQN SSLENVDSRY RNRALMSPAG MLRGDFSLRL FNVTPQDEQK FHCLVLSQSL GFQEVLSVEV TLHVAANFSV PVVSAPHSPS QDELTFTCTS INGYPRPNVY WINKTDNSLL DQALQNDTVF LNMRGLYDVV SVLRIARTPS VNIGCCIENV LLQQNLTVGS QTGNDIGERD KITENPVSTG EKNAATWSIL (SEQ ID NO:343).

In some cases, a T-cell modulatory polypeptide of a multimeric polypeptide is an OX40L polypeptide. In some cases, an OX40L polypeptide of a TMP comprises an amino acid sequence having at least 75%, at least 80%, at least 85%, at least 90%, at least 95%, at least 97%, at least 98%, at least 99%, or 100%, amino acid sequence identity to the following OX40L amino acid sequence: L QVSHRYPRIQ SIKVQFTEYK KEKGFILTSQ KEDEIMKVQN NSVIINCDGF YLISLKGYFS QEVNISLHYQ KDEEPLFQLK KVRSVNSLMV ASLTYKDKVY LNVTTDNTSL DDFHVNGGEL ILIHQNPGEF CVL (SEQ ID NO:344).

In some cases, a T-cell modulatory polypeptide of a multimeric polypeptide is a PD-L2 polypeptide. In some cases, a PD-L2 polypeptide of a multimeric polypeptide comprises an amino acid sequence having at least 75%, at least 80%, at least 85%, at least 90%, at least 95%, at least 97%, at least 98%, at least 99%, or 100%, amino acid sequence identity to amino acids 20-273 of the PD-L2 amino acid sequence: L FTVTVPKELY IIEHGSNVTL ECNFDTGSHV NLGAITASLQ KVENDTSPHR ERATLLEEQL PLGKASFHIP QVQVRDEGQY QCIIIYGVAW DYKYLTLKVK ASYRKINTHI LKVPETDEVE LTCQATGYPL AEVSWPNVSV PANTSHSRTP EGLYQVTSVL RLKPPPGRNF SCVFWNTHVR ELTLASIDLQ SQMEPRTHPT (SEQ ID NO:345).

In some cases, a MOD present in a TMP is a CD80 polypeptide. In some cases, a CD80 polypeptide of a TMP comprises an amino acid sequence having at least 75%, at least 80%, at least 85%, at least 90%, at least 95%, at least 97%, at least 98%, at least 99%, or 100%, amino acid sequence identity to following CD80 amino acid sequence: VIHVTK EVKEVATLSC GHNVSVEELA QTRIYWQKEK KMVLTMMSGD MNIWPEYKNR TIFDITNNLS IVILALRPSD EGTYECVVLK YEKDAFKREH LAEVTLSVKA DFPTPSISDF EIPTSNIRRI ICSTSGGFPE PHLSWLENGE ELNAINTTVS QDPETELYAV SSKLDFNMTT NHSFMCLIKY GHLRVNQTFN WNTTKQEHFP DN (SEQ ID NO:346).

In some cases, a MOD present in a TMP is a CD86 polypeptide. In some cases, a CD86 polypeptide of a TMP comprises an amino acid sequence having at least 75%, at least 80%, at least 85%, at least 90%, at least 95%, at least 97%, at least 98%, at least 99%, or 100%, amino acid sequence identity to the following CD86 amino acid sequence: APLKIQAYFNETADLPCQFANSQNQSLSELVVFWQDQENLVLNEVYLGKEKFDSVHSKYMNRT SFDSDSWTLRLHNLQIKDKGLYQCIIHHKKPTGMIRIHQMNSELSVLANFSQPEIVPISNITENVYI NLTCSSIHGYPEPKKMSVLLRTKNSTIEYDGIMQKSQDNVTELYDVSISLSVSFPDVTSNMTIFCIL ETDKTRLLSSPFSIELEDPQPPPDHIP (SEQ ID NO:347).

In some cases, a MOD present in a TMP is a FasL polypeptide, e.g., the extracellular domain of a FasL polypeptide. In some cases, a FasL polypeptide of a TMP comprises an amino acid sequence having at least 75%, at least 80%, at least 85%, at least 90%, at least 95%, at least 97%, at least 98%, at least 99%, or 100%, amino acid sequence identity to the following FasL extracellular domain amino acid sequence: QLFHLQKE LAELRESTSQ MHTASSLEKQ IGHPSPPPEK KELRKVAHLT GKSNSRSMPL EWEDTYGIVL LSGVKYKKGG LVINETGLYF VYSKVYFRGQ SCNNLPLSHK VYMRNSKYPQ DLVMMEGKMM SYCTTGQMWA RSSYLGAVFN LTSADHLYVN VSELSLVNFE ESQTFFGLYK L (SEQ ID NO:348).

### Scaffold Polypeptides

A TMP can comprise an Fc polypeptide or can comprise another suitable scaffold polypeptide.

Suitable scaffold polypeptides include antibody-based scaffold polypeptides and non-antibody-based scaffolds. Non-antibody-based scaffolds include, e.g., albumin, an XTEN (extended recombinant) polypeptide, transferrin, an Fc receptor polypeptide, an elastin-like polypeptide (see, e.g., Hassouneh et al. (2012) Methods Enzymol. 502:215; e.g., a polypeptide comprising a pentapeptide repeat unit of (Val-Pro-Gly-X-Gly; SEQ ID NO:384), where X is any amino acid other than proline), an albumin-binding polypeptide, a silk-like polypeptide (see, e.g., Valluzzi et al. (2002) Philos Trans R Soc Lond B Biol Sci. 357:165), a silk-elastin-like polypeptide (SELP; see, e.g., Megeed et al. (2002) Adv Drug Deliv Rev. 54:1075), and the like. Suitable XTEN polypeptides include, e.g., those disclosed in WO 2009/023270, WO 2010/091122, WO 2007/103515, US 2010/0189682, and US 2009/0092582; see also Schellenberger et al. (2009) Nat Biotechnol. 27:1186). Suitable albumin polypeptides include, e.g., human serum albumin.

Suitable scaffold polypeptides will in some cases be a half-life extending polypeptides. Thus, in some cases, a suitable scaffold polypeptide increases the in vivo half-life (e.g., the serum half-life) of the TMP, compared to a control TMP lacking the scaffold polypeptide. For example, in some cases, a scaffold polypeptide increases the in vivo half-life (e.g., the serum half-life) of the TMP, compared to a control TMP lacking the scaffold polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold. As an example, in some cases, an Fc polypeptide increases the in vivo half-life (e.g., the serum half-life) of the TMP, compared to a control TMP lacking the Fc polypeptide, by at least about 10%, at least about 15%, at least about 20%, at least about 25%, at least about 50%, at least about 2-fold, at least about 2.5-fold, at least about 5-fold, at least about 10-fold, at least about 25-fold, at least about 50-fold, at least about 100-fold, or more than 100-fold.

### Ig Fc Polypeptides

In some cases, a TMP comprises an Ig Fc polypeptide. An Ig Fc polypeptide is also referred to herein as an “Fc polypeptide.” The Ig Fc polypeptide of a TMP can be a human IgG1 Fc, a human IgG2 Fc, a human IgG3 Fc, a human IgG4 Fc, etc., or a variant of a wild-type Ig Fc polypeptide. Variants include naturally-occurring variants, non-naturally-occurring variants, and combinations thereof.

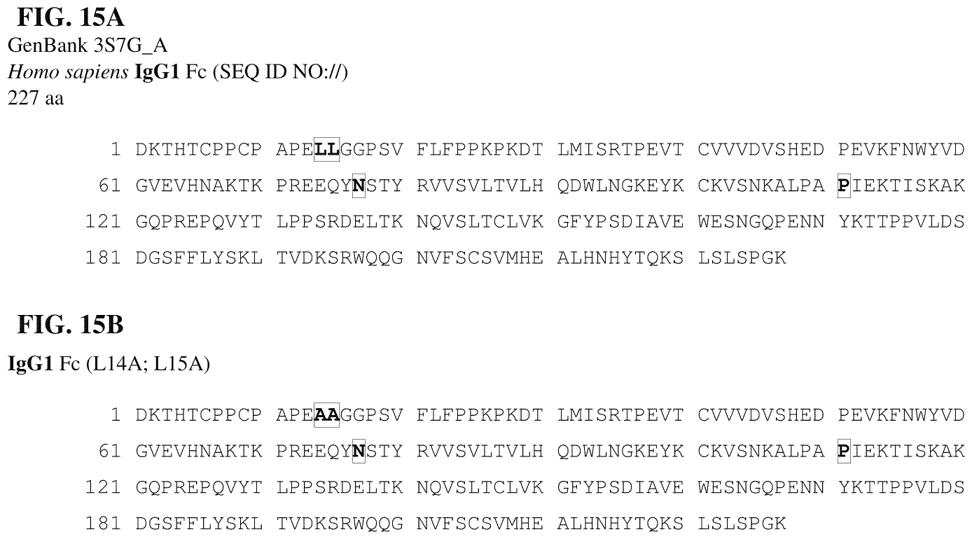

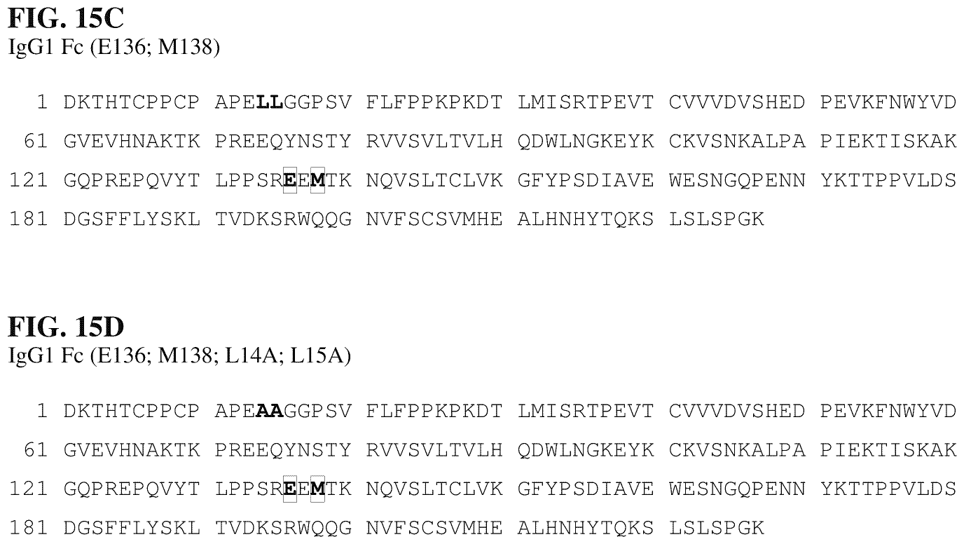

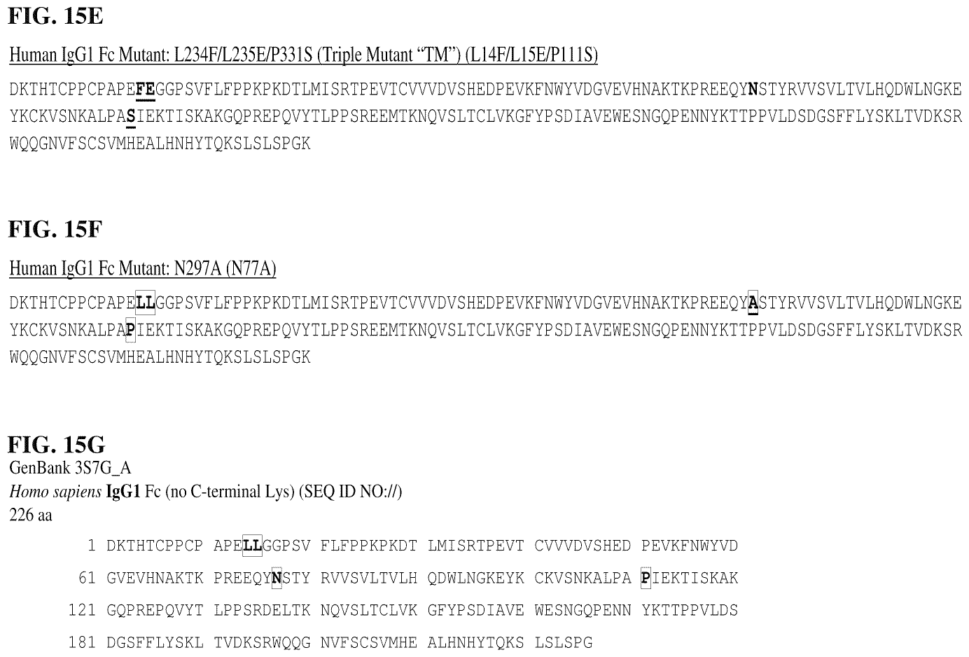

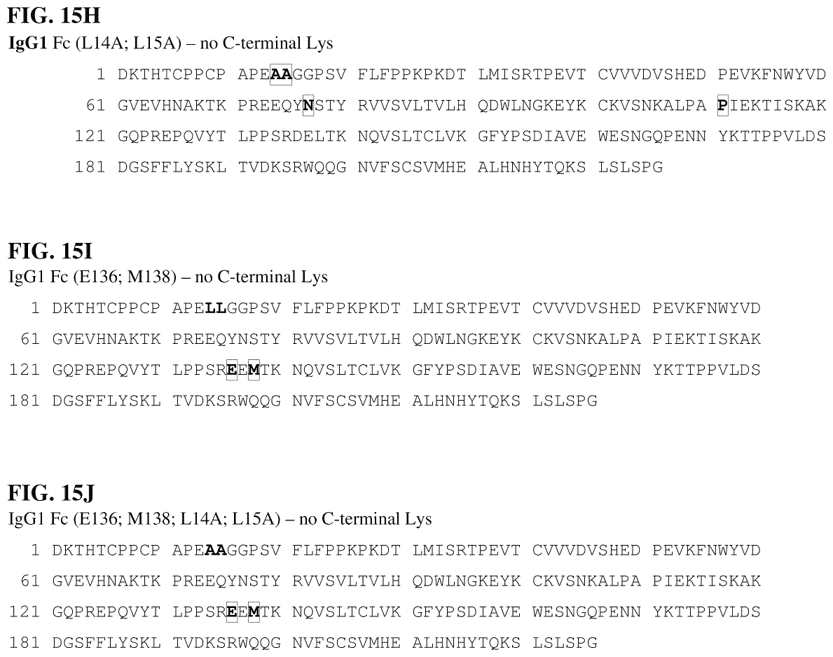

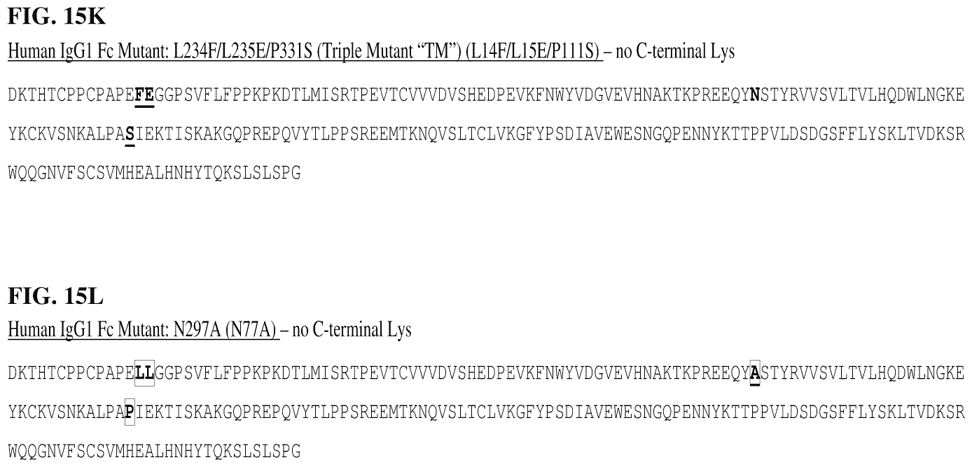

In some cases, the Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the Fc amino acid sequence depicted in any one of FIG. 15A-15L.

In some cases, the Fc polypeptide present in a TMP is an IgG1 Fc polypeptide, or a variant of an IgG1 Fc polypeptide. For example, in some cases, the Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the human IgG1 Fc polypeptide depicted in FIG. 15A. As another example, in some cases, the Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the Fc polypeptide depicted in FIG. 15B; where the Ig Fc polypeptide comprises an Ala at position 14 and an Ala at position 15. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 15A. In some cases, an Fc polypeptide present in a TMP comprises the amino acid sequence depicted in FIG. 15B.

In some cases, the Fc polypeptide present in a TMP is an IgG1 Fc polypeptide, or a variant of an IgG1 Fc polypeptide, where variants include naturally-occurring variants, non-naturally-occurring variants, and combinations thereof. For example, in some cases, the Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the human IgG1 Fc polypeptide depicted in FIG. 15C; where the Ig Fc polypeptide comprises a Glu at position 136 and a Met at position 138. As another example, in some cases, the Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the human IgG1 Fc polypeptide depicted in FIG. 16D; where the Ig Fc polypeptide has Ala at positions 14 and 15; and where the Fc polypeptide comprises a Glu at position 136 and a Met at position 138. In any of the above embodiments, the Ig Fc polypeptide can have an N77 substitution; i.e., the Ig Fc polypeptide can have an amino acid other than Asn at position 77, where in some cases, the Ig Fc polypeptide has an Ala at position 77. In some cases, an Fc polypeptide present in a TMP comprises the amino acid sequence depicted in FIG. 15C. In some cases, an Fc polypeptide present in a TMP comprises the amino acid sequence depicted in FIG. 15D.

In some cases, the Fc polypeptide present in a TMP comprises the amino acid sequence depicted in FIG. 15E (human IgG1 Fc comprising an L234F substitution, an L235E substitution, and a P331S substitution; where L234 corresponds to amino acid 14 of the amino acid sequence depicted in FIG. 15E; L235 corresponds to amino acid 15 of the amino acid sequence depicted in FIG. 15E; and P331 corresponds to amino acid 111 of the amino acid sequence depicted in FIG. 15E). In some cases, the Fc polypeptide present in a TMP comprises the amino acid sequence depicted in FIG. 15F, comprising an N279A substitution (N77A of the amino acid sequence depicted in FIG. 15F).

In some cases, the Ig Fc polypeptide present in a TMP does not include a C-terminal Lys present in a wild-type Ig Fc polypeptide. Thus, for example, in some cases, the Ig Fc polypeptide present in a TMP comprises an amino acid sequence having at least about 70%, at least about 75%, at least about 80%, at least about 85%, at least about 90%, at least about 95%, at least about 97%, at least 98%, at least about 99%, or 100%, amino acid sequence identity to the human IgG1 Fc polypeptide depicted in any one of FIG. 15H-15L, where the Ig Fc polypeptide does not include a C-terminal Lys.

### Linkers

As described above, a pMHC polypeptide present in a TMP comprises a first peptide linker and a second peptide linker. As noted above, a TMP can include one or more additional peptide linkers. For example, the TMP can include independently selected peptide linkers between one or more of: i) the MHC class I heavy chain polypeptide and the MOD; ii) the MHC class I heavy chain polypeptide and the Ig Fc polypeptide; iii) between the MOD and the Ig Fc polypeptide; and iv) where the TMP comprises two MODs in tandem, between the two MODs.

As used herein, the phrase “an optional peptide linker between any two of the components of a TMP” refers to a peptide linker (other than the first peptide linker described above) between any two adjacent polypeptides within the TMP. For example, as used herein, the phrase “an optional peptide linker between any two of the components of a TMP” refers to a peptide linker between one or more of: i) the MHC class I heavy chain polypeptide and the MOD; ii) the MHC class I heavy chain polypeptide and the Ig Fc polypeptide; iii) between the MOD and the Ig Fc polypeptide; and vi) a first MOD and a second MOD. As discussed below, a peptide linker may be a flexible peptide linker, including a short flexible peptide linker, or a rigid peptide linker. As discussed below, a peptide linker may be a rigid peptide linker.

Suitable linkers (also referred to as “spacers”) can be readily selected and can be of any of a number of suitable lengths, such as from 1 amino acid to 25 amino acids, from 3 amino acids to 20 amino acids, from 2 amino acids to 15 amino acids, from 3 amino acids to 12 amino acids, including 4 amino acids to 10 amino acids, 5 amino acids to 9 amino acids, 6 amino acids to 8 amino acids, or 7 amino acids to 8 amino acids. A suitable linker can be 1, 2, 3, 4, 5, 6, 7, 8, 9, 10, 11, 12, 13, 14, 15, 16, 17, 18, 19, 20, 21, 22, 23, 24, or 25 amino acids in length. In some cases, a linker has a length of from 25 amino acids to 50 amino acids, e.g., from 25 to 30, from 30 to 35, from 35 to 40, from 40 to 45, or from 45 to 50 amino acids in length.

### Flexible Peptide Linkers

Exemplary flexible peptide linkers include glycine polymers (G)ₙ, glycine-serine polymers (including, for example, (GS)ₙ, (GSGGS)ₙ (SEQ ID NO:325), (GGGGS)n (SEQ ID NO:326), and (GGGS)ₙ (SEQ ID NO:327), where n is an integer of at least one and can be an integer from 1 to 10), glycine-alanine polymers, alanine-serine polymers, and other flexible peptide linkers known in the art. Glycine and glycine-serine polymers can be used; both Gly and Ser are relatively unstructured, and therefore can serve as a neutral tether between components. Glycine polymers can be used; glycine accesses significantly more phi-psi space than even alanine, and is much less restricted than residues with longer side chains (see Scheraga, Rev. Computational Chem. 11173-142 (1992)). Exemplary flexible peptide linkers can comprise amino acid sequences including, but not limited to, GGSG (SEQ ID NO:349), GGSGG (SEQ ID NO:350), GSGSG (SEQ ID NO:351), GSGGG (SEQ ID NO:352), GGGSG (SEQ ID NO:353), GSSSG (SEQ ID NO:354), GGGGS (SEQ ID NO:355), and the like.

Exemplary flexible peptide linkers include, e.g., (GGGGS)n (SEQ ID NO:328); also referred to as a “G4S” linker), where n is 1, 2, 3, 4, 5, 6, 7, 8, 9, or 10. In some cases, a linker comprises the amino acid sequence (GGGGS)n (SEQ ID NO:328), where n is 1, 2, 3, 4, 5, 6, 7, 8, 9 or 10. In some cases, a linker comprises the amino acid sequence (GGGGS)n (SEQ ID NO:329), where n is 2. In some cases, a linker comprises the amino acid sequence (GGGGS)n (SEQ ID NO:330), where n is 3. In some cases, a linker comprises the amino acid sequence (GGGGS)n (SEQ ID NO:331), where n is 4. In some cases, a linker comprises the amino acid sequence (GGGGS)n (SEQ ID NO:332), where n is 7. In some cases, a linker comprises the amino acid sequence AAAGG (SEQ ID NO:333). Also suitable is a linker having the amino acid sequence AAAGG (SEQ ID NO:333). In some embodiments of a TMP of this disclosure, the β2M polypeptide can be connected to the MHC heavy chain polypeptide by a (GGGGS)n (SEQ ID NO:328) linker, where n is 1, 2, 3, 4, 5, 6, 7, 8, 9, or 10, e.g., where n=2, n=3, n=4, or n=7.

As used in this disclosure, a “short flexible peptide linker” means a flexible peptide linker that comprises fewer than 15 amino acids, i.e., from 2-14 amino acids. For example, a short flexible peptide linker can comprise from 2-4, 2-5, or 3-6 amino acids (e.g., a GGS linker), or from 4-8, 5-10 or from 10-14 amino acids. Within this range includes flexible peptide linkers comprising 2, 3, 4, 5, 6, 7, 8, 9, 10, 11, 12, 13 or 14 amino acids.

### Rigid Peptide Linkers

In some cases, a peptide linker is a rigid peptide linker. As used herein, the term “rigid peptide linker” refers to a linker comprising a contiguous stretch of two or more amino acids that effectively separates protein domains by maintaining a substantially fixed distance/spatial separation between the domains, thereby reducing or substantially eliminating unfavorable interactions between such domains. Rigid peptide linkers are known in the art and generally adopt a relatively well-defined conformation when in solution. Rigid peptide linkers include those which have a particular secondary and/or tertiary structure in solution; and are typically of a length sufficient to confer secondary or tertiary structure to the linker. Rigid peptide linkers include peptide linkers rich in proline, and peptide linkers having an inflexible helical structure, such as an α-helical structure. Rigid peptide linkers are described in, for example, Chen et al. (2013) Adv. Drug Deliv. Rev. 65:1357; and Klein et al. (2014) Protein Engineering, Design & Selection 27:325.

Examples of rigid peptide linkers include, e.g., (EAAAK)n (SEQ ID NO:356), A(EAAAK)n (SEQ ID NO:357), A(EAAAK)nA (SEQ ID NO:358), A(EAAAK)nALEA(EAAAK)nA (SEQ ID NO:359), (Lys-Pro)n (SEQ ID NO:360), (Glu-Pro)n (SEQ ID NO:361), (Thr-Pro-Arg)n (SEQ ID NO:362), and (Ala-Pro)n (SEQ ID NO:363) where n is an integer from 1 to 20 (e.g., n is 1, 2, 3, 4, 5, 6, 7, 8, 9, 10, 11, 12, 13, 14, 15, 16, 17, 18, 19, or 20). Non-limiting examples of suitable rigid peptide linkers comprising EAAAK (SEQ ID NO:364) include EAAAK (SEQ ID NO:364), (EAAAK)₂ (SEQ ID NO:365), (EAAAK)₃ (SEQ ID NO:366), A(EAAAK)₄ALEA(EAAAK)₄A (SEQ ID NO:367), and AEAAAKEAAAKA (SEQ ID NO:368). Non-limiting examples of suitable rigid peptide linkers comprising (AP)n include PAPAP (SEQ ID NO:369); also referred to herein as “(AP)2”); APAPAPAP (SEQ ID NO:370); also referred to herein as “(AP)4”); APAPAPAPAPAP (SEQ ID NO:371); also referred to herein as “(AP)6”); APAPAPAPAPAPAPAP (SEQ ID NO:372); also referred to herein as “(AP)8”); and APAPAPAPAPAPAPAPAPAP (SEQ ID NO:373); also referred to herein as “(AP)10”). Non-limiting examples of suitable rigid peptide linkers comprising (KP)n include KPKP (SEQ ID NO:374); also referred to herein as “(KP)2”); KPKPKPKP (SEQ ID NO:375); also referred to herein as “(KP)4”); KPKPKPKPKPKP (SEQ ID NO:376); also referred to herein as “(KP)6”); KPKPKPKPKPKPKPKP (SEQ ID NO:377); also referred to herein as “(KP)8”); and KPKPKPKPKPKPKPKPKPKP (SEQ ID NO:378); also referred to herein as “(KP)10”). Non-limiting examples of suitable rigid peptide linkers comprising (EP)n include EPEP (SEQ ID NO:379); also referred to herein as “(EP)2”); EPEPEPEP (SEQ ID NO:380); also referred to herein as “(EP)4”); EPEPEPEPEPEP (SEQ ID NO:381); also referred to herein as “(EP)6”); EPEPEPEPEPEPEPEP (SEQ ID NO:382); also referred to herein as “(EP)8”); and EPEPEPEPEPEPEPEPEPEP (SEQ ID NO:383); also referred to herein as “(EP)10”).

Generally speaking, where a TMP comprises a rigid peptide linker and/or a short flexible peptide linker, the TMP can include a rigid peptide linker and/or a short flexible peptide linker between any two of the components of the TMP. One or more rigid peptide linkers and/or short flexible peptide linkers may be used as follows.
(i) In a TMP comprising one or more Position 2 MODs—between one or more of: i) an MHC class I heavy chain polypeptide and a MOD; ii) a MOD and an Ig Fc polypeptide; and iii) where there are multiple MODs in tandem, between a first MOD and a second MOD.
(ii) In a TMP comprising one or more Position 3 MODs—between one or more of: i) an Ig Fc polypeptide and a MOD; and ii) where there are multiple MODs in tandem, between a first MOD and a second MOD.
(iii) In a TMP comprising one or more Position 4 MODs—between one or more of: i) a epitope and a MOD; ii) an MHC class I heavy chain polypeptide and an Ig Fc polypeptide; and iii) where there are multiple MODs in tandem, between a first MOD and a second MOD.

It has been found that, in a TMP having one or more Position 3 MODs, the use of a rigid peptide linker or short flexible peptide linker between the Ig Fc polypeptide and a MOD instead of a flexible peptide linker may enhance the thermal stability of the resulting TMP as compared to a TMP that is identical but for a longer, flexible peptide linker such as a (G4S)3 (i.e., (GGGGS)3) linker (SEQ ID NO:340). While not wishing to be bound by a particular theory, it is believed that the rigid peptide linker or short flexible peptide linker reduces or prevents the interactions of a MOD with other polypeptides within the TMP that can occur with a flexible peptide linker that comprises 15 or more amino acids, resulting in enhanced thermal stability as measured using an accelerated stability assay as described below.

In some cases, the use of a rigid peptide linker or short flexible peptide linker, when interposed between the Ig Fc polypeptide and a MOD of a TMP having one or more Position 3 MODs increases thermal stability, as measured by the 37° C. accelerated thermal stability assay described below, by at least about 10%, at least about 20%, at least about 30%, at least about 40%, at least about 50%, at least about 60%, at least about 70%, at least about 80%, at least about 90%, at least about two-fold, at least about three-fold, at least about four-fold, at least about five-fold, at least about six-fold, at least about seven-fold, at least about eight-fold, or at least about 10-fold, compared to the thermal stability of a control TMP that includes, in place of the rigid peptide linker or short flexible peptide linker, a flexible peptide linker that is a (GGGGS)3 linker (SEQ ID NO:330).

In some cases, the use of a rigid peptide linker or short flexible peptide linker, when interposed between the Ig Fc polypeptide and a MOD of a TMP having one or more Position 3 MODs increases thermal stability, as measured by the 42° C. accelerated thermal stability assay described below, by at least about 10%, at least about 20%, at least about 30%, at least about 40%, at least about 50%, at least about 60%, at least about 70%, at least about 80%, at least about 90%, at least about two-fold, at least about three-fold, at least about four-fold, at least about five-fold, at least about six-fold, at least about seven-fold, at least about eight-fold, or at least about 10-fold, compared to the thermal stability of a control TMP that includes, in place of the rigid peptide linker, or short flexible peptide linker a flexible peptide linker that is a (GGGGS)3 linker (SEQ ID NO:330).

An accelerated thermal stability assay can be carried out as follows. Thermal stability of dimerized TMPs can be assessed using an accelerated stability assay conducted at 4° C., 37° C., and at 42° C. Compositions of dimerized TMPs are kept at the indicated temperatures in a solution (phosphate-buffered saline (PBS) containing 500 mM NaCl, pH 7.4), at a concentration of 10 mg of dimerized TMP/mL solution, for a period of time of 14 days. After 1 day, 7 days, and 14 days, the percent monomer remaining in the solution is determined using size exclusion chromatography. The PBS solution is as follows: 10.14 mM sodium phosphate dibasic, 1.76 mM potassium phosphate monobasic, 2.7 mM KCl, and 0.5 M NaCl; pH 7.4.

Accordingly, this disclosure thus provides methods of increasing the thermal stability of a TMP comprising one or more MODS in Position 2, Position 3, or Position 4.

### Dimerized TMPs

In some cases, a TMP can form a dimer. That is, the present disclosure provides a polypeptide comprising a dimer of two TMPs. The present disclosure thus provides a protein that is a dimerized TMP comprising two TMPs that are covalently linked to each other. The covalent linkage of the dimer can be one or more disulfide bonds, e.g., two disulfide bonds, between an Ig Fc polypeptide in the first TMP and an Ig Fc polypeptide in the second TMP. As but one example, the Ig Fc can be a variant of a human IgG1 Fc polypeptide, which variant has a substantially reduced ability to effect complement-dependent cytotoxicity (CDC) or antibody-dependent cell cytotoxicity (ADCC) (e.g., the Ig Fc polypeptide of FIG. 15B, FIG. 15D, FIG. 15H, or FIG. 15J. When the TMP comprises an Ig Fc polypeptide, the TMP typically will self-assemble into a dimer by spontaneously forming one or more disulfide bonds (typically two disulfide bonds) with the Ig Fc polypeptide of another TMP. Thus, e.g., the Ig Fc polypeptides in the first TMP and the second TMP can be linked to one another by one or more, e.g., two, disulfide bonds. In many cases, the two TMPs will be identical to one another in amino acid sequence and comprise Ig Fc polypeptides that spontaneously form one or more, e.g., two, disulfide bonds, thereby forming a dimerized TMP that is a homodimer.

Accordingly, the present disclosure provides a protein comprising: a) a first TMP; and b) a second TMP, which optionally may be identical to the first TMP, where the first and second TMPs are covalently linked to one another. The covalent linkage can be one or more, e.g., two, disulfide bonds between an Ig Fc polypeptide in the first TMP and an Ig Fc polypeptide in the second TMP.

If desired, the Ig Fc polypeptides of each TMP can comprise interspecific dimerization sequences, e.g., “Knob-in-Hole” sequences that permit two different TMPs to selectively dimerize. Interspecific binding sequences favor formation of heterodimers with their cognate polypeptide sequence (i.e., the interspecific sequence and its counterpart interspecific sequence), particularly those based on Ig Fc sequence variants. Such interspecific polypeptide sequences include Knob-in-Hole, and Knob-in-Hole sequences that facilitate the formation of one or more disulfide bonds. For example, one interspecific binding pair comprises a T366Y and Y407T mutant pair in the CH3 domain interface of IgG1, or the corresponding residues of other immunoglobulins. See Ridgway et al., Protein Engineering 9:7, 617-621 (1996). A second interspecific binding pair involves the formation of a knob by a T366W substitution, and a hole by the triple substitutions T366S, L368A and Y407V on the complementary Ig Fc sequence. See Xu et al. mAbs 7:1, 231-242 (2015). Another interspecific binding pair has a first Fc polypeptide with Y349C, T366S, L368A, and Y407V substitutions and a second Ig Fc polypeptide with S354C, and T366W substitutions (disulfide bonds can form between the Y349C and the S354C). See, e.g., Brinkmann and Konthermann, mAbs 9:2, 182-212 (2015). Ig Fc polypeptide sequences, either with or without knob-in-hole modifications, can be stabilized by the formation of one or more, e.g. two, disulfide bonds between the Ig Fc polypeptides (e.g., the hinge region disulfide bonds). Thus, in some cases, a dimerized TMP can be a heterodimer, comprising two TMP chains that are not identical in amino acid sequence.

Interspecific dimerization sequences also may be employed to enable TMPs to be linked to non-TMP molecules that can provide additional functionality to the TMP. For example, a TMP could be linked to a molecule that comprise polypeptides (e.g., antibodies or binding fragments thereof such as scFvs) that bind to cancer-associated antigens, thereby enabling the TMP to localize to tissues comprising the cancer-associated antigen.

### Additional Polypeptides

A polypeptide chain of a TMP can include one or more polypeptides and conjugate drugs in addition to those described above. Suitable additional polypeptides, including epitope tags and affinity domains, and drug conjugates are described in in published PCT applications WO2020132138A1 and WO2019/051091, discussed above, the disclosures of which as they pertain to epitope tags, affinity domains and drug conjugates are expressly incorporated herein by reference, including specifically paragraphs [00498]-[00508] of WO2020132138A1 and paragraphs [00353]-[00363] of WO2019/051091. The one or more additional polypeptides can be included at the N-terminus of the TMP polypeptide chain, at the C-terminus of the TMP polypeptide chain, or internally within the polypeptide chain of a TMP. As discussed above, additional polypeptides also could be conjugated to TMPs through the use of interspecific sequences.

### Exemplary TMPS and Dimerized TMPS

In the discussion below, the discussion of exemplary TMPs is intended to encompass both TMPs and dimerized TMPs comprising two TMPs where the TMPs are joined by one or more covalent bonds that join the two TMPs, e.g., one, two or more disulfide bonds that spontaneously form between the Ig Fc polypeptides in the two TMPs. Such dimerized TMPs can be either i) homodimers comprising two TMPs, where both of the TMPs have the same amino acid sequence, or ii) heterodimers comprising two TMPs, where the two TMPs differ from one another in amino acid sequence.

In some cases, a TMP comprises the following components: a) pMHC polypeptide, where the pMHC polypeptide comprises, in order from N-terminus to C-terminus: i) a peptide epitope; ii) a first peptide linker comprising a Cys; iii) a β2M polypeptide; iv) a second peptide linker; and v) an MHC class I heavy chain polypeptide, where the MHC class I heavy chain polypeptide comprises a Cys at any one of amino acids 135-143, based on the numbering of the MHC class I heavy chain polypeptide depicted in FIG. 3A, and where amino acid 84, based on the numbering of the MHC class I heavy chain polypeptide depicted in FIG. 3A, is other than Cys; b) one or more MODs; and, optionally c) an Ig Fc polypeptide or a non-Ig scaffold. The TMP may comprise one or more additional independently selected peptide linkers (other than the linkers present in the pMHC), e.g., an independently selected peptide between one or more of: i) the MHC class I heavy chain polypeptide and an Ig Fc polypeptide; ii) the MHC class I heavy chain polypeptide and a MOD; iii) an Ig Fc polypeptide and a MOD; and iv) where the TMP comprises two MODs in tandem, between the two MODs.

In the case of a TMP comprising one or more Position 2 MODs, one or more peptide linkers may be interposed between: i) the MHC class I heavy chain polypeptide and a MOD; ii) a MOD and the Ig Fc polypeptide and the MOD; and iii) where the TMP comprises two or more MODs in tandem, between the MODs. As discussed above, in such TMPs, a flexible peptide linker, a rigid peptide linker or a short flexible peptide linker may be interposed between one or more of: i) an MHC class I heavy chain polypeptide and a MOD; ii) a MOD and an Ig Fc polypeptide; and iii) where there are multiple MODs in tandem, between a first MOD and a second MOD, and so on for additional MODs in tandem. In some cases, the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers (as discussed above) will be interposed between the components that are not connected by a rigid peptide linker or short flexible peptide linker.

In the case of a TMP comprising one or more Position 3 MODs, one or more peptide linkers may be interposed between: i) the MHC class I heavy chain polypeptide and an Ig Fc polypeptide; ii) the Ig Fc polypeptide and the MOD; and iii) where the TMP comprises two or more MODs in tandem, between the MODs. As discussed above, in such TMPs, a flexible peptide linker, a rigid peptide linker or a short flexible peptide linker may be interposed between one or more of: i) an Ig Fc polypeptide and a MOD; and ii) where there are multiple MODs in tandem, between one or more of the MODs, e.g., between a first MOD and a second MOD when there are two MODs in tandem. In some cases, the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the short flexible peptide linkers will comprise from 2-4, 2-5, 3-6, 4-8, 5-10 or 10-14 amino acids. In some cases, the short flexible peptide linker is GGS. Generally speaking, flexible peptide linkers will be interposed between the components that are not connected by a rigid peptide linker or short flexible peptide linker.

As noted above, in some cases, the at least one MOD present in the TMP is a wild-type MOD. In other cases, the at least one MOD present in the TMP is a variant MOD that exhibits reduced affinity for a co-MOD, compared to the affinity of a corresponding wild-type MOD for the co-MOD.

The peptide epitope is a KRAS peptide, where such peptides are as described above.

In the above TMPs, in some cases, the HLA heavy chain that comprises an amino acid sequence having at least 75%, at least 80%, at least 85%, at least 90%, at least 95%, at least 97%, at least 98%, at least 99%, or 100% amino acid sequence identity to an HLA-A*0201 polypeptide, an HLA-A*1101 polypeptide, or HLA-A24 polypeptide, HLA-E polypeptide such as HLA-E*0101 or HLA-E*01.03, or an HLA-G polypeptide such as HLA-G*0101 or and HLA-G*01:04. In some cases, the HLA heavy chain polypeptide is an HLA-A*0301 polypeptide comprising a Cys at position 139. In some cases, the HLA heavy chain polypeptide is an HLA-A*0301 polypeptide comprising an Ala at position 84, a Cys at position 139, and an Ala at position 236. In some cases, the HLA heavy chain polypeptide is an HLA-A*0301 polypeptide comprising an Ala at position 84, a Cys at position 139, and a Cys at position 236. In some cases, the HLA heavy chain polypeptide is an HLA-A*1101 polypeptide comprising a Cys at position 139. In some cases, the HLA heavy chain polypeptide is an HLA-A*1101 polypeptide comprising an Ala at position 84, a Cys at position 139, and an Ala at position 236. In some cases, the HLA heavy chain polypeptide is an HLA-A*1101 polypeptide comprising an Ala at position 84, a Cys at position 139, and a Cys at position 236.

In some cases, a TMP comprises two MODs, where the two MODs have the same amino acid sequence, e.g., the MOD is a variant IL-2 polypeptide that exhibits reduced binding affinity for both the α and β chains of IL2R as compared to wt. IL-2 having a sequence of FIG. 16D, e.g., a variant IL-2 polypeptide comprising H16A and F42A substitutions, H16T and F42A substitutions, H16E and F42A substitutions, or H16D and F42A substitutions.

In some cases, the Ig Fc polypeptide is a variant of a human IgG1 Fc polypeptide that substantially does not induce cell lysis, e.g., an IgG1 Fc polypeptide comprising L234A and L235A (L14A and L15A) substitutions such as is shown in FIG. 16B, FIG. 16D, FIG. 16H, or FIG. 16J.

In some cases, a TMP comprises a MOD at Position 3, wherein the HLA heavy chain polypeptide is a variant HLA-A*0301 polypeptide, e.g., an HLA-A*0301 polypeptide comprising an amino acid sequence having at least 80%, at least 85%, at least 90%, at least 95%, at least 97%, at least 98%, at least 99%, or 100% amino acid sequence identity to an HLA-A*0301 polypeptide of one of FIGS. 7B-7E, where amino acid 139 is a Cys and where amino acid 84 is other than a Cys. In some cases, the variant HLA-A*0301 polypeptide comprises an Ala at position 236. In some cases, the variant HLA-A*0301 polypeptide comprises an Ala at position 84. In some cases, the variant HLA-A*0301 polypeptide comprises a Cys at amino acid 236; and the β2M polypeptide comprises a Cys at amino acid 12. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 15B, FIG. 15D, FIG. 15H, or FIG. 15J, and comprising an amino acid sequence having at least 80%, at least 85%, at least 90%, at least 95%, at least 97%, at least 98%, at least 99%, or 100% amino acid sequence identity to the amino acid sequence depicted in any one of FIG. 15B, FIG. 15D, FIG. 15H, and FIG. 15J. In some cases, the MOD is a variant IL-2 polypeptide that exhibits reduced binding affinity for both the α and β chains of IL2R as compared to wild-type IL-2 having the amino acid sequence depicted in FIG. 16D, e.g., a variant IL-2 polypeptide comprising H16A and F42A substitutions, H16T and F42A substitutions, H16D and F42A substitutions or H16E and F42A substitutions. In some cases, the β2M polypeptide present in a TMP comprises an amino acid sequence having at least 80%, at least 85%, at least 90%, at least 95%, at least 97%, at least 98%, at least 99%, or 100% amino acid sequence identity to the β2M amino acid sequence depicted in FIG. 2A. In some cases, the β2M polypeptide present in a TMP comprises an amino acid sequence having at least 80%, at least 85%, at least 90%, at least 95%, at least 97%, at least 98%, at least 99%, or 100% amino acid sequence identity to the β2M amino acid sequence depicted in FIG. 2B and comprises a Cys at amino acid 12. The peptide epitope is a KRAS peptide, where such peptides are described above. In some cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises a rigid peptide linker between: a) an Ig Fc polypeptide present in the TMP; and b) the first variant IL-2 MOD, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises a rigid peptide linker between: a) an Ig Fc polypeptide present in the TMP; and b) the first variant IL-2 MOD, where the rigid peptide linker comprises the amino acid sequence (AP)n, where n=1, 2, 3, or 4.

In some cases, a TMP comprises a MOD at Position 3, wherein the HLA heavy chain polypeptide is a variant HLA-A*1101 polypeptide, e.g., an HLA-A*0301 polypeptide comprising an amino acid sequence having at least 80%, at least 85%, at least 90%, at least 95%, at least 97%, at least 98%, at least 99%, or 100% amino acid sequence identity to an HLA-A*1101 polypeptide of one of FIGS. 4B-4E, where amino acid 139 is a Cys and where amino acid 84 is other than a Cys. In some cases, the variant HLA-A*1101 polypeptide comprises an Ala at position 236. In some cases, the variant HLA-A*0301 polypeptide comprises an Ala at position 84. In some cases, the variant HLA-A*1101 polypeptide comprises a Cys at amino acid 236; and the β2M polypeptide comprises a Cys at amino acid 12. In some cases, the Ig Fc polypeptide is a variant of human IgG1 Fc polypeptide that substantially does not cause cell lysis, e.g., a human IgG1 Fc polypeptide comprising L234A and L235A substitutions as shown in FIG. 16B, FIG. 16D, FIG. 16H, or FIG. 16J, and comprising an amino acid sequence having at least 80%, at least 85%, at least 90%, at least 95%, at least 97%, at least 98%, at least 99%, or 100% amino acid sequence identity to the amino acid sequence depicted in any one of FIG. 16B, FIG. 16D, FIG. 16H, and FIG. 16J. In some cases, the MOD is a variant IL-2 polypeptide that exhibits reduced binding affinity for both the α and β chains of IL2R as compared to wild-type IL-2 having the amino acid sequence depicted in FIG. 16D, e.g., a variant IL-2 polypeptide comprising H16A and F42A substitutions, H16T and F42A substitutions, H16E and F42A substitutions, or H16D and F42A substitutions. In some cases, the β2M polypeptide present in a TMP comprises an amino acid sequence having at least 80%, at least 85%, at least 90%, at least 95%, at least 97%, at least 98%, at least 99%, or 100% amino acid sequence identity to the β2M amino acid sequence depicted in FIG. 2A. In some cases, the β2M polypeptide present in a TMP comprises an amino acid sequence having at least 80%, at least 85%, at least 90%, at least 95%, at least 97%, at least 98%, at least 99%, or 100% amino acid sequence identity to the β2M amino acid sequence depicted in FIG. 2B and comprises a Cys at amino acid 12. The peptide epitope is a KRAS peptide, where such peptides are described above. In some cases, the TMP comprises a rigid peptide linker between a variant IL-2 MOD and an Ig Fc polypeptide present in the TMP, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises two copies of the variant IL-2 MOD. In some cases, the TMP comprises a rigid peptide linker between: a) an Ig Fc polypeptide present in the TMP; and b) the first variant IL-2 MOD, where the rigid peptide linker comprises an amino acid sequence selected from EAAAK (SEQ ID NO:356), A(EAAAK)n (SEQ ID NO:357), A(EAAAK)nA (SEQ ID NO:358), (AP)n (SEQ ID NO:363), (EP)n (SEQ ID NO:361), and (KP)n (SEQ ID NO:360), where n is an integer from 1 to 10 (e.g., n is 1, 2, 3, 4, 5, 6, 7, 8, 9, or 10). In some cases, the TMP comprises a rigid peptide linker between: a) an Ig Fe polypeptide present in the TMP; and b) the first variant IL-2 MOD, where the rigid peptide linker comprises the amino acid sequence (AP)n, where n=1, 2, 3, or 4.

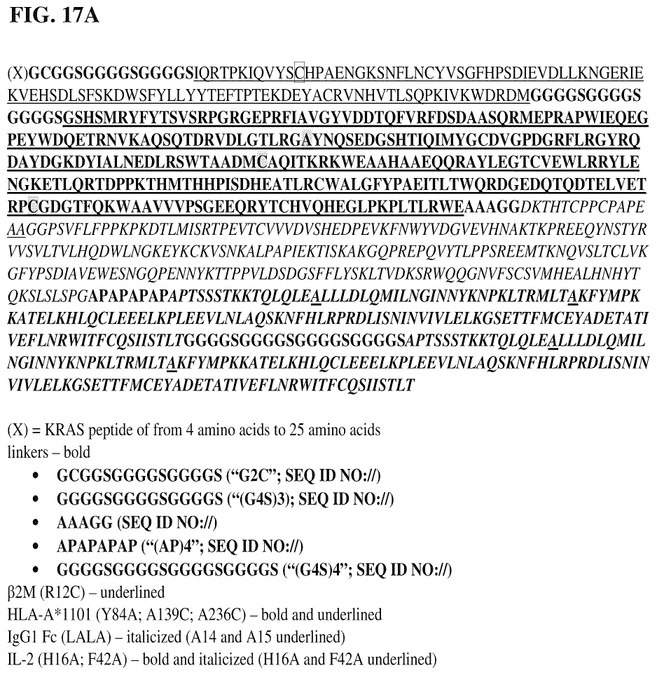

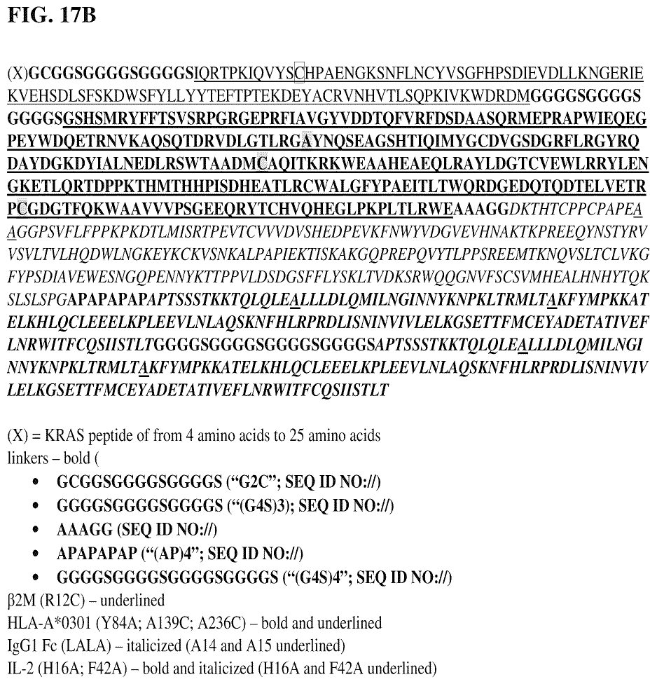

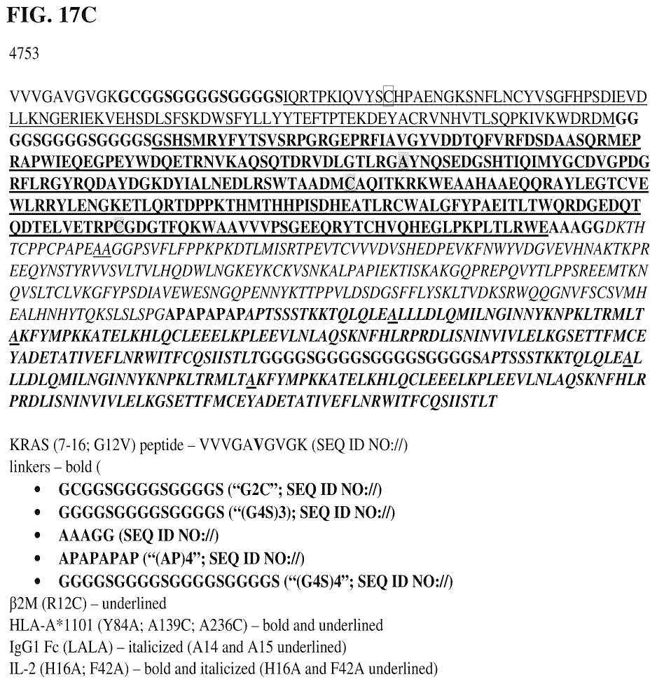

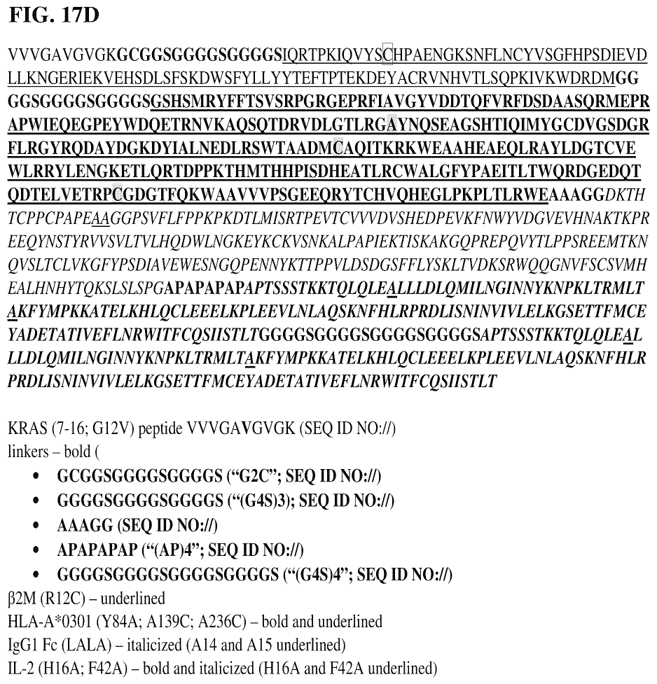

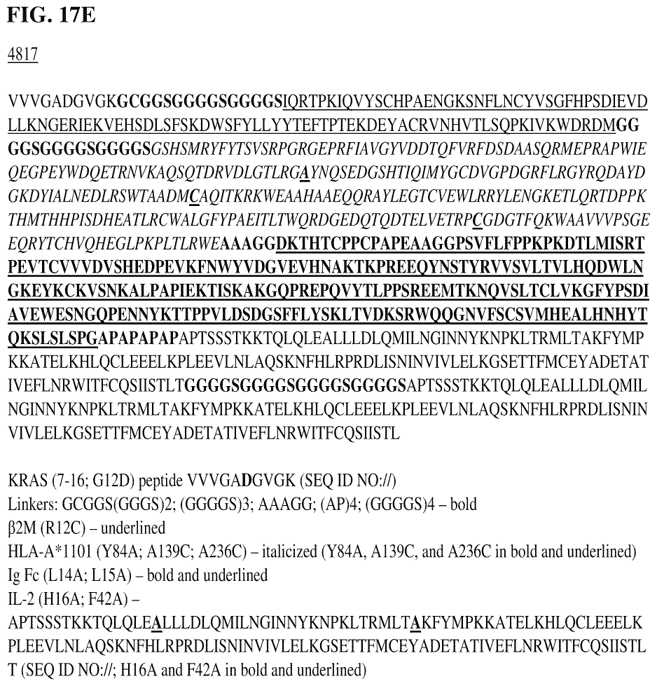

In some cases, a TMP comprises an amino acid sequence having at least 80%, at least 85%, at least 90%, at least 95%, at least 97%, at least 98%, or at least 99%, amino acid sequence identity to the amino acid sequence depicted in FIG. 17A, where X is a KRAS peptide epitope having a length of from 4 amino acids to 25 amino acids. In some cases, X has a length of from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, X is a peptide of from 8 to 12 amino acids in length. In some cases, X is a peptide of 8, 9, 10, or 11 amino acids in length. The peptide epitope is a KRAS peptide, where such peptides are described above.

In some cases, a TMP comprises an amino acid sequence having at least 80%, at least 85%, at least 90%, at least 95%, at least 97%, at least 98%, or at least 99%, amino acid sequence identity to the amino acid sequence depicted in FIG. 17B, where X is a peptide epitope having a length of from 4 amino acids to 25 amino acids. In some cases, X has a length of from 4-20 aa (e.g., 4 amino acids (aa), 5 aa, 6 aa, 7 aa, 8 aa, 9 aa, 10 aa, 11 aa, 12 aa, 13 aa, 14 aa, 15 aa, 16 aa, 17 aa, 18 aa, 19 aa or 20 aa), including a range of from 6-15 aa, 8-12 aa, 8-16 aa, 8-10 aa, 9-11 aa, 5-10 aa, 10-15 aa, and 15-20 aa in length. In some cases, X is a peptide of from 8 to 12 amino acids in length. In some cases, X is a peptide of 8, 9, 10, or 11 amino acids in length. The peptide epitope is a KRAS peptide, where such peptides are described above.

In some cases, a TMP comprises an amino acid sequence having at least 80%, at least 85%, at least 90%, at least 95%, at least 97%, at least 98%, at least 99%, or 100%, amino acid sequence identity to the amino acid sequence depicted in FIG. 17C.

In some cases, a TMP comprises an amino acid sequence having at least 80%, at least 85%, at least 90%, at least 95%, at least 97%, at least 98%, at least 99%, or 100%, amino acid sequence identity to the amino acid sequence depicted in FIG. 17D.

### Nucleic Acids, Recombinant Expression Vectors, and Modified Host Cells

The present disclosure provides a nucleic acid comprising a nucleotide sequence encoding a TMP of the present disclosure. In some cases, the nucleotide sequence encoding the TMP is operably linked to one or more transcriptional control elements. In some cases, the transcriptional control element is a promoter that is functional in a eukaryotic cell. In some cases, the nucleic acid is present in a recombinant expression vector.

The present disclosure thus provides recombinant expression vectors comprising nucleic acids encoding a TMP. In some cases, the recombinant expression vector is a non-viral vector. In some cases, the recombinant expression vector is a viral construct, e.g., a recombinant adeno-associated virus construct (see, e.g., U.S. Pat. No. 7,078,387), a recombinant adenoviral construct, a recombinant lentiviral construct, a recombinant retroviral construct, a non-integrating viral vector, etc.

Suitable expression vectors are well known to persons skilled in the art. See, e.g., published PCT application WO2020132138A1 and WO2019/051091, the disclosures of which as they pertain to such expression vectors are expressly incorporated herein by reference, including specifically paragraphs [00515]-[00520] of WO2020132138A1 and paragraphs [00401]-[00406] of WO2019/051091.

The present disclosure further provides a genetically modified host cell, where the host cell is genetically modified with a nucleic acid or expression vector as described above.

Suitable host cells include eukaryotic cells, such as yeast cells, insect cells, and mammalian cells. In some cases, the host cell is a cell of a mammalian cell line. Suitable mammalian cell lines include human cell lines, non-human primate cell lines, rodent (e.g., mouse, rat) cell lines, and the like. Suitable mammalian cell lines include, but are not limited to, HeLa cells (e.g., American Type Culture Collection (ATCC) No. CCL-2), CHO cells (e.g., ATCC Nos. CRL9618, CCL61, CRL9096), 293 cells (e.g., ATCC No. CRL-1573), Vero cells, NIH 3T3 cells (e.g., ATCC No. CRL-1658), Huh-7 cells, BHK cells (e.g., ATCC No. CCL10), PC12 cells (ATCC No. CRL1721), COS cells, COS-7 cells (ATCC No. CRL1651), RAT1 cells, mouse L cells (ATCC No. CCLI.3), human embryonic kidney (HEK) cells (ATCC No. CRL1573), HLHepG2 cells, and the like.

In some cases, the host cell is a mammalian cell that has been genetically modified such that it does not synthesize endogenous MHC β2M.

In some cases, the host cell is a mammalian cell that has been genetically modified such that it does not synthesize endogenous MHC class I heavy chain. In some cases, the host cell is a mammalian cell that has been genetically modified such that it does not synthesize endogenous MHC β2M and such that it does not synthesize endogenous MHC class I heavy chain.

### Methods of Generating a T-Cell Modulatory Polypeptide

A TMP of the present disclosure can be generated by culturing a genetically modified host cell of the present disclosure in a suitable culture medium in vitro, where such culturing results in production of the TMP. For example, a mammalian host cell (e.g., a CHO cell) can be genetically modified with a recombinant expression vector comprising a nucleotide sequence encoding a TMP of the present disclosure; and the genetically modified mammalian host cell can be cultured in vitro in a suitable culture medium, such that the genetically modified mammalian host cell produces the TMP. The TMP can be isolated, e.g., from the culture medium in which the genetically modified mammalian host cell is cultured and/or from a cell lysate of the genetically modified mammalian host cell. The TMP can be isolated using any of a variety of well-established methods. Where the TMP comprises an Ig Fc polypeptide at its C terminus, intracellular processing may remove a C-terminal Lys residue from the C-terminus of the Ig Fc polypeptide; see, e.g., van den Bremer et al. (2015) mAbs 7:4; and Sissolak et al. (2019) J. Industrial Microbiol. & Biotechnol. 46:1167. And as noted above, two TMPs that each comprise an Ig Fc polypeptide (e.g., an IgG1 Fc) may spontaneously form a homodimer of the two TMPs, wherein the individual TMPs are joined by one or more disulfide bonds between their respective Ig Fc portions.

### Compositions

The present disclosure provides compositions, including pharmaceutical compositions, comprising a TMP or dimerized TMP as disclosed herein. The present disclosure provides compositions, including pharmaceutical compositions, comprising a nucleic acid or a recombinant expression vector.

### Compositions Comprising a TMP or Dimerized TMP

A composition can comprise, in addition to a TMP or dimerized TMP, one or more pharmaceutically acceptable additives, a variety of which are known in the art and need not be discussed in detail herein. See, for example, the ninth (or latest) edition of Sheskey et al., “Handbook of Pharmaceutical Excipients” (2020), and/or the 23^{rd} (or latest) edition of “Remington: The Science and Practice of Pharmacy”, 23rd Ed. (2020).

Where a TMP or dimerized TMP is administered as an injectable (e.g. subcutaneously, intraperitoneally, intramuscularly, and/or intravenously) directly into a tissue, a formulation can be provided as a ready-to-use dosage form that may be directly injected or infused into the patient or admixed with a saline solution for infusion, or possibly as a non-aqueous form (e.g., a reconstitutable storage-stable powder) or aqueous form, such as liquid composed of pharmaceutically acceptable carriers and excipients. Formulations may also be provided so as to enhance serum half-life of the TMP following administration. For example, the TMP or dimerized TMP may be provided in a liposome formulation, prepared as a colloid, or other conventional techniques for extending serum half-life. The preparations may also be provided in controlled release or slow-release forms.

The concentration of a TMP or dimerized TMP in a liquid composition formulation can vary widely (e.g., from less than about 0.1%, usually at or at least about 2% to as much as 20% to 50% or more by weight). Included within this range is a concentration of from about 5 to about 15 mg/mL, from about 8 to about 12 mg/mL, from about 9 to about 11 mg/mL, including about 5 mg/mL, about 6 mg/mL, about 7 mg/mL, about 8 mg/mL, about 9 mg/mL, about 10 mg/mL, about 11 mg/mL, about 12 mg/mL, about 13 mg/mL, about 14 mg/mL and about 15 mg/mL. The concentration may depend on numerous factors, including the stability of the TMP in the liquid composition.

In some cases, a TMP or dimerized TMP is present in a liquid composition. In some cases, a composition comprises: a) a TMP or dimerized TMP; and b) saline (e.g., 0.9% NaCl). In some cases, the composition is sterile and suitable for administration to a human subject.

### Methods of Modulating T Cell Activity

The present disclosure provides a method of selectively modulating the activity of an epitope-specific T cell (e.g., a T cell specific for a peptide epitope present in a TMP, such as a KRAS peptide), the method comprising contacting the T cell with a TMP or dimerized TMP, where contacting the T cell with a TMP or dimerized TMP selectively modulates the activity of the epitope-specific T cell. In some cases, the contacting occurs in vitro. In some cases, the contacting occurs in vivo.

Where a TMP or dimerized TMP includes a MOD that is an activating polypeptide, and the peptide is a cancer-associated peptide, contacting the T cell with the TMP or dimerized TMP activates the epitope-specific T cell, increasing the cytotoxic activity of the T cell toward a cancer cell expressing the cancer-associated peptide epitope and/or increasing the number of the epitope-specific T cells.

In some cases, a TMP or dimerized TMP, when administered to an individual in need thereof, induces both an epitope-specific T cell response and an epitope non-specific T cell response. In other words, in some cases, a TMP, when administered to an individual in need thereof (i) induces an epitope-specific T cell response by modulating the activity of a first T cell that displays both a TCR specific for the peptide epitope present in the TMP and a co-MOD that binds to the MOD present in the TMP; and (ii) induces an epitope non-specific T cell response by modulating the activity of a second T cell that displays a TCR specific for an epitope other than the peptide epitope present in the TMP, and a co-MOD that binds to the MOD present in the TMP. The ratio of the epitope-specific T cell response to the epitope-non-specific T cell response is at least 2:1, at least 5:1, at least 10:1, at least 15:1, at least 20:1, at least 25:1, at least 50:1, or at least 100:1. The ratio of the epitope-specific T cell response to the epitope-non-specific T cell response is from about 2:1 to about 5:1, from about 5:1 to about 10:1, from about 10:1 to about 15:1, from about 15:1 to about 20:1, from about 20:1 to about 25:1, from about 25:1 to about 50:1, or from about 50:1 to about 100:1, or more than 100:1. This ratio is determined by measuring the increase in the number of T cells specific for the target peptide epitope (e.g., KRAS peptide) versus the increase in the number of T cells that are not specific for that target epitope. For example, conventional flow cytometry methods may be employed. “Modulating the activity” of a T cell can include one or more of the following when an activating MOD such as a variant IL-2 is present: i) activating a cytotoxic (e.g., CD8⁺) T cell; ii) inducing cytotoxic activity of a cytotoxic (e.g., CD8⁺) T cell; iii) inducing production and release of a cytotoxin (e.g., a perforin; a granzyme; a granulysin) by a cytotoxic (e.g., CD8⁺) T cell; iv) inducing proliferation of a cytotoxic (e.g., CD8⁺) T cell.

As discussed above, in some cases, a TMP or dimerized TMP, when administered to an individual in need thereof, induces a proliferation of epitope-specific T cells. The increase in the percentage of epitope-specific T cells can be measured by conventional flow cytometry methods. Thus, e.g., the percent of total CD8+ T cells that are specific for the peptide epitope may be increased following contact with the TMP by at least about 10%, at least about 15%, at least about 20%, at least about 25%, at least about 50%, at least about 75%, at least about 2-fold, at least about 2.5-fold, at least about 3-fold, at least about 4-fold, or higher than 4-fold.

The present disclosure provides a method of delivering a MOD selectively to target T cell, the method comprising contacting a mixed population of T cells with a TMP or dimerized TMP, where the mixed population of T cells comprises the target T cell and non-target T cells, where the target T cell is specific for the peptide epitope present within the TMP or dimerized TMP, and where the contacting step delivers the one or more MODs present within the TMP or dimerized TMP to the target T cell. In some cases, the population of T cells is in vitro. In some cases, the population of T cells is in vivo in an individual. In some cases, the method comprises administering the TMP or dimerized TMP to the individual. In some cases, the target T cell is a cytotoxic T cell. In some cases, the mixed population of T cells is an in vitro population of mixed T cells obtained from an individual, and the contacting step results in activation and/or proliferation of the target T cell, generating a population of activated and/or proliferated target T cells; in some of these instances, the method further comprises administering the population of activated and/or proliferated target T cells to the individual.

The present disclosure provides a method of detecting, in a mixed population of T cells obtained from an individual, the presence of a target T cell that binds an epitope of interest (e.g., a KRAS epitope), the method comprising: a) contacting in vitro the mixed population of T cells with a TMP or dimerized TMP, wherein the TMP or dimerized TMP comprises the peptide epitope of interest; and b) detecting activation and/or proliferation of T cells in response to said contacting, wherein activated and/or proliferated T cells indicates the presence of the target T cell.

### Treatment Methods

This disclosure provides a method of treatment of an individual, the method comprising administering to the individual an amount of: i) a TMP or dimerized TMP, ii) one or more nucleic acids encoding the TMP, or iii) a B cell or other blood cell comprising the TMP, effective to treat the individual. Also provided is a TMP or dimerized TMP for use in a method of treatment of the human or non-human animal body. In some cases, a treatment method of this disclosure comprises administering to an individual in need thereof one or more nucleic acids or recombinant expression vectors comprising nucleotide sequences encoding a TMP or dimerized TMP. In some cases, the one or more nucleic acids or recombinant expression vectors are present in cells (e.g., B cells or other blood cells) that are administered to the individual, which cells are then capable of producing the TMP or dimerized TMP in vivo. In some cases, a treatment method of this disclosure comprises administering to an individual in need thereof one or more nucleic acids (e.g., mRNA molecules) comprising nucleotide sequences encoding a TMP or dimerized TMP, or a cell, e.g., a B cell or other blood cell such as a red blood cell comprising one or more of such nucleic acids. In some cases, a treatment method comprises administering to an individual in need thereof a TMP or dimerized TMP. Conditions that can be treated include, e.g., cancer.

### Treating Cancer

A TMP of the present disclosure can be administered to an individual in need thereof to treat a cancer in the individual, where the cancer expresses, or overexpresses, a protein comprising the KRAS peptide present in the TMP.

The present disclosure provides a method of treating cancer in an individual, the method comprising administering to the individual an effective amount of: i) a TMP or dimerized TMP, ii) one or more nucleic acids encoding the TMP, or iii) a B cell or other blood cell comprising the TMP, effective to treat the individual where the TMP or dimerized TMP displays a T-cell epitope, and where the TMP or dimerized TMP comprises an activating MOD. In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor, as discussed below), reduces the overall tumor burden in the individual, i.e., the amount of cancer in the body, or alternatively, causes the total tumor burden in the patient to remain relatively stable for a sufficient period of time for the patient to have a confirmed “stable disease”, a confirmed “partial response”, or a confirmed “complete response” as determined by standard RECIST criteria or iRECIST criteria.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), reduces the number of cancer cells in the individual, including to substantially undetectable levels.

In some cases, an effective amount of a TMP or dimerized TMP is an amount that, when administered in one or more doses to an individual in need thereof (an individual having a tumor), either as a monotherapy or as part of a combination therapy (e.g., as part of a combination therapy with an immune checkpoint inhibitor), reduces the tumor volume in the individual. For example, in some cases, an effective amount of a TMP or dimerized TMP is an amount that, when administered in one or more doses to an individual in need thereof (an individual having a tumor), either as a monotherapy or as part of a combination therapy (e.g., as part of a combination therapy with an immune checkpoint inhibitor), reduces the tumor volume in the individual by at least 10%, at least 15%, at least 20%, at least 25%, at least 30%, at least 40%, at least 50%, at least 60%, at least 70%, at least 80%, at least 90%, or at least 95%, compared to the tumor volume in the individual before administration of the TMP or dimerized TMP, or in the absence of administration with the TMP or dimerized TMP. Tumor volume is determined using the formula (length×width×width)/2, where length represents the largest tumor diameter and width represents the perpendicular tumor diameter.

In some cases, an effective amount of a TMP or dimerized TMP is an amount that, when administered in one or more doses to an individual in need thereof, increases survival time of the individual. For example, in some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), increases survival time of the individual by at least 1 month, at least 2 months, at least 3 months, from 3 months to 6 months, from 6 months to 1 year, from 1 year to 2 years, from 2 years to 5 years, from 5 years to 10 years, or more than 10 years, compared to the expected survival time of the individual in the absence of administration with the TMP or dimerized TMP.

In some cases, an effective amount of a TMP or dimerized TMP is an amount that, when administered in one or more doses to an individual in need thereof, either as a monotherapy or as part of a combination therapy (e.g., as part of a combination therapy with an immune checkpoint inhibitor), reduces the level of circulating tumor DNA (“ctDNA”) in the patient by at least 10%, at least 15%, at least 20%, at least 25%, at least 30%, at least 40%, at least 50%, at least 60%, at least 70%, at least 80%, at least 90%, or at least 95%, compared to the ctDNA levels in the individual before administration of the TMP or dimerized TMP, or in the absence of administration with the TMP or dimerized TMP. The level of ctDNA can be determined using any known method; see, e.g., Cescon et al. (2020) Nature Cancer 1:276.

Cancers that can be treated with a method of this disclosure include cancers in which the cancer cells express a mutated form of KRAS. Examples include adenocarcinomas and hematological malignancies. Examples of cancers that can be treated with a method of this disclosure include multiple myeloma; B-cell lymphoma; breast cancer; lung cancer; ovarian carcinoma; pancreatic cancer; colorectal cancer; prostate cancer; renal cancer; acute myelogenous leukemia; mesothelioma; thyroid cancer; head and neck cancer; stomach cancer; urothelial cancer; cervical cancer; and ovarian endometrial cancer.

### Dosages

A suitable dosage of a TMP or dimerized TMP can be determined by an attending physician or other qualified medical personnel, based on various clinical factors. As is well known in the medical arts, dosages for any one patient depend upon many factors, including the patient's size, body surface area, age, the particular polypeptide or nucleic acid to be administered, sex of the patient, time, and route of administration, general health, and other drugs being administered concurrently. A TMP or dimerized TMP may be administered in amounts between 0.1 mg/kg body weight and 20 mg/kg body weight per dose, e.g. between 0.1 mg/kg body weight to 10 mg/kg body weight, e.g. between 0.5 mg/kg body weight to 5 mg/kg body weight, between 1 mg/kg body weight to 5 mg/kg body weight; between 5 mg/kg body weight to 10 mg/kg body weight; between 10 mg/kg body weight to 15 mg/kg body weight; between 15 mg/kg body weight to 20 mg/kg body weight, however, doses above this exemplary range are envisioned, especially considering the aforementioned factors. If the regimen is a continuous infusion, it can also be in the range of 1 g to 10 mg per kilogram of body weight per minute.

Where the TMP includes one or more variant IL-2 MODs, or where a dimerized TMP comprises 2 or more (e.g., 4) variant IL-2 MODS discussed above such as the variants that comprise H16 and F42 substitutions, exemplary amounts of TMP or dimerized TMP include from 1 mg/kg body weight to 5 mg/kg body weight, from 5 mg/kg body weight to 10 mg/kg body weight, from about 1 mg/kg body weight to about 5 mg/kg body weight, and from about 5 mg/kg body weight to about 10 mg/kg body weight. When the TMP comprises four of such variant IL-2 MODs, exemplary amounts of TMP or dimerized TMP include 1 mg/kg, 2 mg/kg, 3 mg/kg, 4 mg/kg or 5 mg/kg, e.g., 2 mg/kg, 3 mg/kg or 4 mg/kg.

Persons of ordinary skill in the art can easily estimate repetition rates for dosing based on measured residence times and concentrations of the administered agent in bodily fluids or tissues. Following successful treatment, it may be desirable to have the patient undergo maintenance therapy to prevent the recurrence of the disease state, wherein a TMP or dimerized TMP is administered in maintenance doses, ranging from about 1 mg/kg body weight to about 5 mg/kg body weight (e.g., 1-5 mg/kg), from about 5 mg/kg body weight to about 10 mg/kg body weight (e.g., 5-10 mg/kg), from about 10 mg/kg body weight to about 15 mg/kg body weight (e.g., 10-15 mg/kg), from about 15 mg/kg body weight to about 20 mg/kg body weight (e.g., 15-20 mg/kg), or amounts exceeding 20 mg/kg of body weight.

Those of skill will readily appreciate that dose levels can vary as a function of the specific TMP or dimerized TMP, the severity of the symptoms and the susceptibility of the subject to side effects. Preferred dosages for a given compound are readily determinable by those of skill in the art by a variety of means.

In some cases, multiple doses of a TMP or dimerized TMP, a nucleic acid, or a recombinant expression vector (or a cell, e.g., a B cell or other blood cell such as a red blood cell comprising a nucleic acid or recombinant expression vector) are administered. The frequency of administration of a TMP or dimerized TMP, a nucleic acid, or a recombinant expression vector can vary depending on any of a variety of factors, e.g., severity of the symptoms, etc. For example, in some cases, a TMP or dimerized TMP, a nucleic acid, or a recombinant expression vector (or a cell, e.g., a B cell or a blood cell such as a red blood cell comprising a nucleic acid or recombinant expression vector) is administered once per month, twice per month, three times per month, every other week (qow), once per week (qw), once every two weeks, once every three weeks, or once every four weeks. It also is possible to administer a TMP more often than once per week, e.g., twice per week (biw), three times per week (tiw), four times per week, five times per week, six times per week, every other day (qod), or daily (qd), including more often than once per day, e.g., twice a day (qid), or three times a day (tid). Where the TMP or dimerized TMP is administered intravenously, administration once every week, once every two weeks, once every three weeks or once every four weeks or once every month may be commonly employed at the beginning of treatment. When a TMP is administered with an immune checkpoint inhibitor (CPI), e.g., an anti-PD1 CPI such pembrolizumab, nivolumab, cemiplimab or durvalumab, then it may be desirable to administer the TMP on the same schedule as the CPI, e.g., once every three weeks. For example, pembrolizumab is administered once every three weeks.

The duration of administration of a TMP or dimerized TMP, a nucleic acid, or a recombinant expression vector (or a cell, e.g., a B cell or a blood cell such as a red blood cell comprising a nucleic acid or recombinant expression vector), e.g., the period of time over which a TMP or dimerized TMP, a nucleic acid, or a recombinant expression vector is administered, can vary, depending on any of a variety of factors, e.g., patient response, etc. For example, a TMP or dimerized TMP, a nucleic acid, or a recombinant expression vector (or a cell, e.g., a B cell or a blood cell such as a red blood cell comprising a nucleic acid or recombinant expression vector) can be administered over a period of time ranging from about one day to about one week, two weeks, from about two weeks to about four weeks, from about one month to about two months, from about two months to about four months, from about four months to about six months, from about six months to about eight months, from about eight months to about 1 year, from about 1 year to about 2 years, or from about 2 years to about 4 years, or more. The length of time may depend on whether the TMP is administered in a neoadjuvant setting, in which case it may be administered only once or a few times, e.g., two, three or four times, for only a week or a few weeks before a main treatment such as surgery. In an adjuvant setting or a setting in which the patient is being treated for a cancer that has newly appeared or is recurrent or metastatic, the treatment duration may continue indefinitely or until the patient exhibits progressive disease as determined by standard RECIST or iRECIST criteria.

### Routes of Administration

An active agent (a TMP or dimerized TMP, a nucleic acid, or a recombinant expression vector, or a cell, e.g., a B cell or other blood cell such as a red blood cell comprising a nucleic acid or recombinant expression vector) is administered to an individual using any available method and route suitable for systemic and localized routes of administration.

A TMP or dimerized TMP of this disclosure typically will be delivered via intravenous administration, but other conventional and pharmaceutically acceptable routes of administration may be used, including intratumoral, peritumoral, intramuscular, or subcutaneous. Other routes of administration also are possible, including intralymphatic, intratracheal, intracranial, intradermal, topical application, intraarterial, rectal, nasal, oral, and other enteral and parenteral routes of administration. Routes of administration may be combined, if desired, or adjusted depending upon the TMP or dimerized TMP and/or the desired effect. A TMP or dimerized TMP, or a nucleic acid or recombinant expression vector, or a cell, e.g., a B cell or a blood cell such as a red blood cell comprising a nucleic acid or recombinant expression vector, can be administered in a single dose or in multiple doses.

### Combination Therapy

A TMP or dimerized TMP can be administered to an individual in need thereof in combination with one or more additional therapeutic agents or therapeutic treatment. A suitable dosage of the TMP or dimerized TMP typically will be the same as the dosage amount for monotherapy with the TMP (described above) or may be less or more than the monotherapy dose. Suitable additional therapeutic agents include, e.g.: i) an immune checkpoint inhibitor; ii) a cancer chemotherapeutic agent; and iii) one or more additional TMPs or dimerized TMPs. Suitable additional therapeutic treatments include, e.g., radiation, surgery (e.g., surgical resection of a tumor), and the like.

A TMP or dimerized TMP can be administered to an individual in need thereof at the same time, or at different times, as the one or more additional therapeutic agent is administered.

As noted above, a treatment method can comprise co-administration of a TMP or dimerized TMP and an immune checkpoint inhibitor such as an antibody specific for an immune checkpoint. By “co-administration” is meant that both a TMP or dimerized TMP and an antibody specific for an immune checkpoint are administered to an individual, although not necessarily at the same time, in order to achieve a therapeutic effect that is the result of having administered both the TMP or dimerized TMP and the immune checkpoint inhibitor. The administration of the TMP or dimerized TMP and the antibody specific for an immune checkpoint can be substantially simultaneous, e.g., the TMP or dimerized TMP can be administered to an individual within about 1 minute to about 24 hours (e.g., within about 1 minute, within about 5 minutes, within about 15 minutes, within about 30 minutes, within about 1 hour, within about 2 hours, within about 4 hours, within about 8 hours, within about 12 hours, or within about 24 hours) of administration of the antibody specific for an immune checkpoint. Alternatively, the TMP or dimerized TMP and immune checkpoint inhibitor can be administered on different schedules, including different days and different weeks, and different frequencies. In some cases, a TMP or dimerized TMP is administered to an individual who is undergoing treatment with, or who has undergone treatment with, an antibody specific for an immune checkpoint.

Exemplary immune checkpoint inhibitors include inhibitors that target immune checkpoint polypeptide such as CD27, CD28, CD40, CD122, CD96, CD73, CD47, OX40, GITR, CSF1R, JAK, PI3K delta, PI3K gamma, TAM, arginase, CD137 (also known as 4-1BB), ICOS, A2AR, B7-H3, B7-H4, BTLA, CTLA-4, LAG3, TIM3, VISTA, CD96, TIGIT, CD122, PD-1, PD-L1 and PD-L2. In some cases, the immune checkpoint polypeptide is a stimulatory checkpoint molecule selected from CD27, CD28, CD40, ICOS, OX40, GITR, CD122, and CD137. In some cases, the immune checkpoint polypeptide is an inhibitory checkpoint molecule selected from A2AR, B7-H3, B7-H4, BTLA, CTLA-4, IDO, KIR, LAG3, PD-1, TIM3, CD96, TIGIT, and VISTA.

In some cases, the immune checkpoint inhibitor is an antibody specific for an immune checkpoint. Suitable anti-immune checkpoint antibodies include, but are not limited to, nivolumab (Bristol-Myers Squibb), pembrolizumab (Merck), pidilizumab (Curetech), AMP-224 (GlaxoSmithKline/Amplimmune), MPDL3280A (Roche), MDX-1105 (Medarex, Inc./Bristol Myer Squibb), MEDI-4736 (Medimmune/AstraZeneca), arelumab (Merck Serono), ipilimumab (YERVOY, (Bristol-Myers Squibb), tremelimumab (Pfizer), pidilizumab (CureTech, Ltd.), IMP321 (Immutep S.A.), MGA271 (Macrogenics), BMS-986016 (Bristol-Meyers Squibb), lirilumab (Bristol-Myers Squibb), urelumab (Bristol-Meyers Squibb), PF-05082566 (Pfizer), IPH2101 (Innate Pharma/Bristol-Myers Squibb), MEDI-6469 (MedImmune/AZ), CP-870,893 (Genentech), Mogamulizumab (Kyowa Hakko Kirin), Varlilumab (CellDex Therapeutics), Avelumab (EMD Serono), Galiximab (Biogen Idec), AMP-514 (Amplimmune/AZ), AUNP 12 (Aurigene and Pierre Fabre), Indoximod (NewLink Genetics), NLG-919 (NewLink Genetics), INCB024360 (Incyte); KN035; and combinations thereof. For example, in some cases, the immune checkpoint inhibitor is an anti-PD-1 antibody. Suitable anti-PD-1 antibodies include, e.g., nivolumab, pembrolizumab (also known as MK-3475), pidilizumab, SHR-1210, PDR001, AMP-224, cemiplimab and durvalumab. In some cases, the anti-PD-1 monoclonal antibody is nivolumab, pembrolizumab, cemiplimab, durvalumab or PDR001. Suitable anti-PD1 antibodies are described in U.S. Patent Publication No. 2017/0044259. For pidilizumab, see, e.g., Rosenblatt et al. (2011) J. Immunother. 34:409-18. In some cases, the immune checkpoint inhibitor is an anti-CTLA-4 antibody. In some cases, the anti-CTLA-4 antibody is ipilimumab or tremelimumab. For tremelimumab, see, e.g., Ribas et al. (2013) J. Clin. Oncol. 31:616-22. In some cases, the immune checkpoint inhibitor is an anti-PD-L1 antibody. In some cases, the anti-PD-L1 monoclonal antibody is BMS-935559, MEDI4736, MPDL3280A (also known as RG7446), KN035, or MSB0010718C. In some embodiments, the anti-PD-L1 monoclonal antibody is MPDL3280A (atezolizumab) or MEDI4736 (durvalumab). For durvalumab, see, e.g., WO 2011/066389. For atezolizumab, see, e.g., U.S. Pat. No. 8,217,149.

Among such checkpoint inhibitors, antibodies to PD-1, PD-L1 and CTLA-4 are the most common, with at least nivolumab, tremelimumab, pembrolizumab, ipilimumab, cemiplimab, atezolizumab, avelumab, tisleizumab and durvalumab having been approved by the FDA and/or regulatory agencies outside of the U.S. The TMPs and dimerized TMPs of this disclosure also may be co-administered with combinations of checkpoint inhibitors, e.g., a combination of (i) an antibody to PD-1 or PD-L1, and (ii) an antibody to CTLA-4.

In some cases, the at least one additional therapeutic agent comprises one or more additional TMPs or dimerized TMPs. In some cases, the method comprises administering to an individual in need thereof: a) a first composition comprising a first TMP; and b) a second composition comprising a second TMP, where the second TMP is a TMP that is different from the first TMP, e.g., comprising a different peptide epitope and/or one or more different MODs.

### Subjects Suitable for Treatment

Subjects suitable for treatment with a method of the present disclosure include individuals who have cancer, including individuals who have been diagnosed as having cancer, individuals who have been treated for cancer but who failed to respond to the treatment, and individuals who have been treated for cancer and who initially responded but subsequently became refractory to the treatment. Subjects suitable for treatment include individuals having a cancer in which the cancer cells express, or overexpress, a cancer-associated KRAS peptide.

In some cases, the subject is an individual who is undergoing treatment with an immune checkpoint inhibitor. In some cases, the subject is an individual who has undergone treatment with an immune checkpoint inhibitor, but whose disease has progressed despite having received such treatment. In some cases, the subject is an individual who is undergoing treatment with, or who has undergone treatment with, a cancer chemotherapeutic agent. In some cases, the subject is an individual who is preparing to undergo treatment with, is undergoing treatment with, or who has undergone treatment with, an immune checkpoint inhibitor. In some cases, the subject is an individual who is preparing to undergo treatment with, is undergoing treatment with, or who has undergone treatment with, a cancer chemotherapeutic agent, radiation treatment, surgery, and/or treatment with another therapeutic agent.

### Detection Methods

A TMP or dimerized TMP is useful for diagnostic applications and therapeutic applications. As discussed below, when used for diagnostic applications, a TMP or dimerized TMP also can comprise a detectable label so that binding of the TMP or dimerized TMP to a target T cell is detected by detecting the detectable label.

The present disclosure thus provides a method of detecting the presence and/or activation of an antigen-specific T-cell. The methods comprise contacting a T cell with a TMP or dimerized TMP of the present disclosure; and detecting binding of the TMP or dimerized TMP to the T cell, and/or activation of the T cell. The present disclosure provides a method of detecting an antigen-specific T cell, the method comprising contacting a T cell with a TMP or dimerized TMP of the present disclosure, wherein binding of the TMP or dimerized TMP to the T cell indicates that the T cell is specific for the peptide epitope present in the TMP or dimerized TMP, that is, the T cell comprises a T cell receptor that is specific for the peptide epitope present in the TMP or dimerized TMP.

In some cases, the TMP or dimerized TMP comprises a detectable label. Suitable detectable labels include, but are not limited to, a radioisotope, a fluorescent polypeptide, or an enzyme that generates a fluorescent product, and an enzyme that generates a colored product. Where the TMP or dimerized TMP comprises a detectable label, binding of the TMP or dimerized TMP to the T cell is detected by detecting the detectable label.

In some cases, a TMP or dimerized TMP comprises a detectable label suitable for use in in vivo imaging, e.g., suitable for use in positron emission tomography (PET), single photon emission tomography (SPECT), near infrared (NIR) optical imaging, x-ray imaging, computer-assisted tomography (CAT), or magnetic resonance imaging (MRI), or other in vivo imaging method. Examples of suitable labels for in vivo imaging include gadolinium chelates (e.g., gadolinium chelates with DTPA (diethylenetriamine penta-acetic acid), DTPA-bismethylamide (BMA), DOTA (dodecane tetraacetic acid), or HP-DO3A (1,4,7-tris(carboxymethyl)-10-(2′-hydroxypropyl)-1,4,7,10-tetraazacycl ododecane)), iron chelates, magnesium chelates, manganese chelates, copper chelates, chromium chelates, iodine-based materials, and radionuclides. Suitable radionuclides include, but are not limited to, ¹²³I, ¹²⁵I, ¹³⁰I, ¹³¹I, ¹³³I, ¹³⁵I, ⁴⁷Sc, ⁷²As, ⁷²Se, ⁹⁰Y, ⁸⁸Y, ⁹⁷Ru, ¹⁰⁰Pd, ¹⁰¹mRh, ¹¹⁹Sb, ¹²⁸Ba, ¹⁹⁷Hg, ²¹¹At, ²¹²Bi, ²¹²Pb, ¹⁰⁹Pd, ¹¹¹In, ⁶⁷Ga, ⁶⁸Ga, ⁶⁴Cu, ⁶⁷Cu, ⁷⁵Br, ⁷⁷Br, ⁹⁹mTc, ¹⁴C, ¹³N, ¹⁵O, ³²P, ³³P, and ¹⁸F. In some cases, the detectable label is a positron-emitting isotope such as ¹¹C, ¹³N, ¹⁵O, ¹⁸F, ⁶⁴Cu, ⁶⁸Ga, ⁷⁸Br, ⁸²Rb, ⁸⁶Y, ⁹⁰Y, ²²Na, ²⁶Al, ⁴⁰K, ⁸³Sr, ⁸⁹Zr, or ¹²⁴I. In some cases, the detectable label is ⁶⁴Cu. See, e.g., Woodham, Andrew et al., In vivo detection of antigen-specific CD8+ T cells by immuno-positron emission tomography, Nature Methods Articles (2020) https://doi.org/10.1038/s41592-020-0934-5.

Suitable fluorescent proteins include, but are not limited to, green fluorescent protein (GFP) or variants thereof, blue fluorescent variant of GFP (BFP), cyan fluorescent variant of GFP (CFP), yellow fluorescent variant of GFP (YFP), enhanced GFP (EGFP), enhanced CFP (ECFP), enhanced YFP (EYFP), GFPS65T, Emerald, Topaz (TYFP), Venus, Citrine, mCitrine, GFPuv, destabilised EGFP (dEGFP), destabilized ECFP (dECFP), destabilized EYFP (dEYFP), mCFPm, Cerulean, T-Sapphire, CyPet, YPet, mKO, HcRed, t-HcRed, DsRed, DsRed2, DsRed-monomer, J-Red, dimer2, t-dimer2(12), mRFP1, pocilloporin, Renilla GFP, Monster GFP, paGFP, Kaede protein and kindling protein, Phycobiliproteins and Phycobiliprotein conjugates including B-Phycoerythrin, R-Phycoerythrin and Allophycocyanin. Other examples of fluorescent proteins include mHoneydew, mBanana, mOrange, dTomato, tdTomato, mTangerine, mStrawberry, mCherry, mGrape1, mRaspberry, mGrape2, mPlum (Shaner et al. (2005) Nat. Methods 2:905-909), and the like. Any of a variety of fluorescent and colored proteins from Anthozoan species, as described in, e.g., Matz et al. (1999) Nature Biotechnol. 17:969-973, is suitable for use.

Suitable enzymes include, but are not limited to, horse radish peroxidase (HRP), alkaline phosphatase (AP), beta-galactosidase (GAL), glucose-6-phosphate dehydrogenase, beta-N-acetylglucosaminidase, β-glucuronidase, invertase, Xanthine Oxidase, firefly luciferase, glucose oxidase (GO), and the like.

In some cases, binding of a TMP or dimerized TMP to a T cell is detected using a detectably labeled antibody specific for the TMP or dimerized TMP. An antibody specific for the TMP or dimerized TMP can comprise a detectable label such as a radioisotope, a fluorescent polypeptide, or an enzyme that generates a fluorescent product, or an enzyme that generates a colored product.

In some cases, the T cell being detected is present in a sample comprising a plurality of T cells. For example, a T cell being detected can be present in a sample comprising from 10 to 109 T cells, e.g., from 10 to 10², from 10² to 10⁴, from 10⁴ to 10⁶, from 10⁶ to 10⁷, from 10⁷ to 10⁸, or from 10⁸ to 10⁹, or more than 10⁹, T cells.

### HLA/Peptide Binding Assays

Whether a given peptide (e.g., a peptide that comprises a cancer-associated epitope) binds a class I HLA (comprising an HLA heavy chain and a β2M polypeptide), and, when bound to the HLA complex, can effectively present an epitope to a TCR, can be determined using any of a number of well-known methods. Assays include binding assays and T-cell activation assays, including cell-based binding assays, biochemical binding assays, T-cell activation assays, ELISPOT assays, cytotoxicity assays and Detection of Antigen-specific T cells with peptide-HLA tetramers. Such assays are described in the published scientific literature as well as in published PCT application WO2020132138A1, the disclosure of which as it pertains to specific binding assays is expressly incorporated herein by reference, including specifically paragraphs [00217]-[00225].

As another example, multimers (e.g., tetramers) of peptide-HLA complexes are generated with fluorescent or heavy metal tags. The multimers can then be used to identify and quantify specific T cells via flow cytometry (FACS) or mass cytometry (CyTOF). Detection of epitope-specific T cells provides direct evidence that the peptide-bound HLA molecule is capable of binding to a specific TCR on a subset of antigen-specific T cells. See, e.g., Klenerman et al. (2002) Nature Reviews Immunol. 2:263.

### Examples of Non-Limiting Aspects of the Disclosure

Aspects, including embodiments, of the present subject matter described above may be beneficial alone or in combination, with one or more other aspects or embodiments. Without limiting the foregoing description, certain non-limiting aspects of the disclosure are provided below. As will be apparent to those of skill in the art upon reading this disclosure, each of the individually numbered aspects may be used or combined with any of the preceding or following individually numbered aspects. This is intended to provide support for all such combinations of aspects and is not limited to combinations of aspects explicitly provided below:

Aspect 1. A single-chain T-cell modulatory polypeptide (TMP) comprising (i) a KRAS peptide-major histocompatibility complex (KRAS pMHC), (ii) one or more immunomodulatory polypeptides, and (iii) an immunoglobulin (Ig) Fc polypeptide or a non-immunoglobulin scaffold component, wherein the KRAS pMHC comprises in order from N-terminus to C-terminus:
a) a KRAS peptide comprising a KRAS epitope expressed on a cancer cell, wherein the KRAS peptide has a length of from about 7 amino acids to about 16 amino acids, optionally from 8 amino acids to 12 amino acids;
b) a first peptide linker, wherein the first peptide linker comprises a cysteine (Cys);
c) a beta-2 microglobulin (β2M) polypeptide;
d) a second peptide linker; and
e) a major histocompatibility complex (MHC) class I heavy chain polypeptide, wherein the MHC class I heavy chain polypeptide comprises a Cys at any one of amino acids 135-143, based on the numbering of the MHC class I heavy chain polypeptide depicted in FIG. 3A, and wherein amino acid 84, based on the numbering of the MHC class I heavy chain polypeptide depicted in FIG. 3A, is other than Cys,
wherein the pMHC polypeptide comprises a disulfide bond formed between the Cys present in the first peptide linker and the Cys at any one of amino acids 135-143 of the MHC class I heavy chain polypeptide, and wherein the KRAS pMHC polypeptide presents the peptide for binding to a T cell receptor.

Aspect 2. The TMP of aspect 1, wherein the MHC class I heavy chain polypeptide comprises a Cys at amino acid 138, 139, or 140 based on the numbering of the MHC class I heavy chain polypeptide depicted in FIG. 3A.

Aspect 3. The TMP of aspect 1, wherein the MHC class I heavy chain polypeptide comprises a Cys at amino acid 139 based on the numbering of the MHC class I heavy chain polypeptide depicted in FIG. 3A.

Aspect 4. The TMP of any one of aspects 1-3, wherein the peptide has a length of from 8 amino acids to 12 amino acids.

Aspect 5. The TMP of any one of aspects 1-4, wherein the first linker comprises the sequence CGGGS(GGGGS)n, GCGGS(GGGGS)n, or GGCGS(GGGGS)n, wherein n is an integer from 1-10.

Aspect 6. The TMP of any one of aspects 1-4, wherein the first linker comprises the sequence GCGGS(GGGGS)n, wherein n is an integer from 1-4.

Aspect 7. The TMP of any one of aspects 1-4, wherein the first linker comprises the sequence GCGGS(GGGGS)n, wherein n is 2.

Aspect 8. The TMP of any one of aspects 1-7, wherein the β2M polypeptide comprises an amino acid sequence having at least 95% amino acid sequence identity to the β2M amino acid sequence depicted in FIG. 2A.

Aspect 9. The TMP of any one of aspects 1-8, wherein the β2M polypeptide comprises a Cys at amino acid 12, wherein the MHC class I heavy chain polypeptide comprises a Cys at amino acid 236, and wherein the TMP further comprises a disulfide bond formed between the Cys at amino acid 12 of the β2M polypeptide and the Cys at amino acid 236 of the MHC class I heavy chain polypeptide.

Aspect 10. The TMP of any one of aspects 1-9, wherein the MHC class I heavy chain polypeptide comprises an amino acid sequence having at least 95% amino acid sequence identity to an HLA-A polypeptide, an HLA-B polypeptide, an HLA-C polypeptide, or an HLA-E polypeptide.

Aspect 11. The TMP of aspect 10, wherein the HLA-A polypeptide is an HLA-A*0301, an HLA-A*1101 polypeptide, an HLA-A*3303 polypeptide, an HLA-A*0201 polypeptide, or an HLA-A*2401 polypeptide.

Aspect 12. The TMP of any one of aspects 1-11, wherein the MHC class I heavy chain polypeptide comprises an amino acid sequence having at least 95% amino acid sequence identity to the amino acid sequence depicted in any one of FIG. 3B-3E, FIG. 4B-4E, FIG. 5B-5E, FIG. 6B-6E, FIG. 7B-7E, FIG. 11B-11E, or FIG. 12B-12E.

Aspect 13. The TMP of any one of aspects 1-12, wherein the second peptide linker has a length of from 4 amino acids to 25 amino acids.

Aspect 14. The TMP of any one of aspects 1-13, wherein the KRAS peptide comprises a sequence selected from the group consisting of:
A) VVGADGVGK (SEQ ID NO:478), VVGACGVGK (SEQ ID NO:479), VVGAVGVGK (SEQ ID NO:480), VVVGADGVGK (SEQ ID NO:481), VVVGAVGVGK (SEQ ID NO:482), VVVGACGVGK (SEQ ID NO:483), VTGADGVGK (SEQ ID NO:484), VTGAVGVGK (SEQ ID NO:485), VTGACGVGK (SEQ ID NO:486), VTVGADGVGK (SEQ ID NO:487), VTVGAVGVGK (SEQ ID NO:488), and VTVGACGVGK (SEQ ID NO:489); and wherein the KRAS peptide has a length of 9 amino acids or 10 amino acids, or a length of at least 9 amino acids or 10 amino acids;
B) VVVGAGDVGK (SEQ ID NO:490); VVGAGDVGK (SEQ ID NO:491); VVVGARGVGK (SEQ ID NO:492); and VVGARGVGK (SEQ ID NO:493); and wherein the KRAS peptide has a length of 9 amino acids or 10 amino acids, or a length of at least 9 amino acids or 10 amino acids;
C) LVVVGADGV (SEQ ID NO:494), LVVVGAVGV (SEQ ID NO:495), LVVVGACGV (SEQ ID NO:496), KLVVVGADGV (SEQ ID NO:497), KLVVVGAVGV (SEQ ID NO:498), KLVVVGACGV (SEQ ID NO:499), LLVVGADGV (SEQ ID NO:500), LLVVGAVGV (SEQ ID NO:501), LLVVGACGV (SEQ ID NO:502), FLVVVGADGV (SEQ ID NO:503), FLVVVGAVGV (SEQ ID NO:504), and FLVVVGACGV (SEQ ID NO:505); and wherein the KRAS peptide has a length of 9 amino acids or 10 amino acids, or a length of at least 9 amino acids or 10 amino acids;
D) KLVVVGAGDV (SEQ ID NO:418); and KLVVVGARGV (SEQ ID NO:419); wherein the KRAS peptide has a length of 9 amino acids or 10 amino acids;
E) GAGDVGKSAL (SEQ ID NO:420); AGDVGKSAL (SEQ ID NO:421); DVGKSALTI (SEQ ID NO:422); GAVGVGKSAL (SEQ ID NO:423); AVGVGKSAL (SEQ ID NO:424); YKLVVVGAV (SEQ ID NO:425); ARGVGKSAL (SEQ ID NO:426); GARGVGKSAL (SEQ ID NO:427); EYKLVVVGAR (SEQ ID NO:428); RGVGKSALTI (SEQ ID NO:429); LVVVGARGV (SEQ ID NO:430); GADGVGKSAL (SEQ ID NO:431); ACGVGKSAL (SEQ ID NO:432); and GACGVGKSAL (SEQ ID NO:433); wherein the KRAS peptide has a length of 9 amino acids or 10 amino acids; and
F) VVGAVGVGK (SEQ ID NO:466), VVVGAVGVGK (SEQ ID NO:467), VGAVGVGKS (SEQ ID NO:468), VGAVGVGKSA (SEQ ID NO:469), AVGVGKSAL (SEQ ID NO:470), AVGVGKSALT (SEQ ID NO:471), GAVGVGKSAL (SEQ ID NO:472), GAVGVGKSA (SEQ ID NO:473), LVVVGAVGVG (SEQ ID NO:474), LVVVGAVGV (SEQ ID NO:475), KLVVVGAVGV (SEQ ID NO:476), and KLVVVGAVG (SEQ ID NO:477); where the KRAS peptide has a length of 9 amino acids or 10 amino acids.

Aspect 15. The TMP of any one of aspects 1-13, wherein:
A) the KRAS peptide is KLVVVGADGV (SEQ ID NO:409) and the MHC heavy chain polypeptide comprises an amino acid sequence having at least 95% amino acid sequence identity to an HLA-A*0201 polypeptide; or
B) the KRAS peptide is VVVGADGVGK (SEQ ID NO:436) or VVGAVGVGK (SEQ ID NO:438), and the MHC heavy chain polypeptide comprises an amino acid sequence having at least 95% amino acid sequence identity to an HLA-A11*01 polypeptide.

Aspect 16. The TMP of any one of aspects 1-15, wherein the TMP comprises from N-terminus to C-terminus:

a1) the pMHC, b1) the one or more immunomodulatory polypeptides, and c1) an Ig Fc polypeptide; or a2) the pMHC, b2) an Ig Fc polypeptide, and c2) the one or more immunomodulatory polypeptides, and wherein the TMP may further comprise an independently selected linker interposed between any two of the components of the TMP.

Aspect 17. The TMP of aspect 16, wherein the TMP comprises from N-terminus to C-terminus:
a1) the pMHC, b1) an optional linker, c1) an Ig Fc polypeptide, d1) an optional linker, and e1) an immunomodulatory polypeptide; or
a2) the pMHC, b2) an optional linker, c2) the Ig Fc polypeptide, d2) an optional linker, e2) a first immunomodulatory polypeptide, f2) an optional linker, and g2) a second immunomodulatory polypeptide.

Aspect 18. The TMP of any one of aspects 1-17, wherein the TMP comprises at least one rigid peptide linker, and wherein each rigid peptide linker is independently selected from the group consisting of: i) (AP)n, where n is an integer from 1-10; ii) (EP)n, where n is an integer from 1-10; iii) (KP)n, where n is an integer from 1-10; and iv) a peptide comprising EAAAK.

Aspect 19. The TMP of any one of aspects 1-18, wherein the TMP comprises an Ig Fc polypeptide, optionally wherein the Ig Fc polypeptide substantially does not induce cell lysis.

Aspect 20. The TMP of aspect 19, wherein the Ig Fc polypeptide comprises an Ala at amino acid 14 and an Ala at amino acid 15, based on the amino acid number of the Ig Fc amino acid sequence depicted in FIG. 15A.

Aspect 21. The TMP of aspect 19 or 20, wherein the Ig Fc polypeptide comprises an amino acid sequence having at least 95% amino acid sequence identity to the amino acid sequence depicted in FIG. 15H.

Aspect 22. The TMP of any one of aspects 1-21, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

Aspect 23. The TMP of aspect 22, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

Aspect 24. The TMP of aspect 23, wherein the at least one immunomodulatory polypeptide comprises i) an H16A substitution and an F42A substitution; ii) an H16T substitution and an F42A substitution, iii) an H16D and F42A substitution, or iv) an H16E substitution and an F42A substitution.

Aspect 25. A protein comprising two TMPs according to any one of aspects 1-24, wherein the two TMPs each comprise and Ig Fc polypeptide, wherein the two TMPs have the same amino acid sequence, and wherein the two TMPs are joined by one or more disulfide bonds that join the Ig Fc component of one TMP to the Ig Fc component of the other TMP.

Aspect 26. A protein according to aspect 25, wherein the two TMPs are joined by two disulfide bonds that join the Ig Fc component of one TMP to the Ig Fc component of the other TMP.

Aspect 27. A pharmaceutical composition comprising the TMP of any one of aspects 1-24, or the protein of aspect 25 or 26.

Aspect 28. A nucleic acid comprising a nucleotide sequence encoding the TMP according to any one of aspects 1-26.

Aspect 29. The nucleic acid of aspect 28, wherein the nucleotide sequence is operably linked to one or more transcriptional control elements.

Aspect 30. A recombinant expression vector comprising the nucleic acid of aspect 28 or aspect 29.

Aspect 31. A host cell genetically modified with the nucleic acid of aspect 28 or aspect 29, or the recombinant expression vector of aspect 30.

Aspect 32. A method of producing a TMP according to any one of aspects 1-24, or the protein of aspect 25 or 26, the method comprising culturing in vitro the host cell of aspect 31, wherein the host cell produces the TMP.

Aspect 33. A method of modulating the activity of a T cell, the method comprising contacting the T cell with a TMP according to any one of aspects 1-24 or the protein of aspect 25 or 26.

Aspect 34. A method of aspect 33, wherein said contacting is in vitro.

Aspect 35. A method of aspect 33, wherein said contacting is in vivo.

Aspect 36. A method of treating a KRAS-associated cancer in an individual, the method comprising administering to the individual an effective amount of the TMP of any one of aspects 1-24 or the protein of aspect 25 or 26.

Aspect 37. The method of aspect 36, further comprising administering one or more additional therapeutic agents.

Aspect 38. The method of aspect 37, wherein the one or more additional therapeutic agents comprises an immune checkpoint inhibitor.

Aspect 39. The method of aspect 38, wherein the immune checkpoint inhibitor is an antibody specific for PD-1, PD-L1, or CTLA4.

Aspect. 40, The method of aspect 39, wherein the antibody is a single-chain Fv or a nanobody.

### EXAMPLES

The following examples are put forth so as to provide those of ordinary skill in the art with a complete disclosure and description of how to make and use the present disclosure, and are not intended to limit the scope of the disclosure, nor are they intended to represent that the experiments below are all or the only experiments performed. Efforts have been made to ensure accuracy with respect to numbers used (e.g., amounts, temperature, etc.) but some experimental errors and deviations should be accounted for. Unless indicated otherwise, parts are parts by weight, temperature is in degrees Celsius, and pressure is at or near atmospheric. Standard abbreviations may be used, e.g., bp, base pair(s); kb, kilobase(s); pl, picoliter(s); s or see, second(s); min, minute(s); h or hr, hour(s); aa, amino acid(s); kb, kilobase(s); bp, base pair(s); nt, nucleotide(s); i.m., intramuscular(ly); i.p., intraperitoneal(ly); s.c., subcutaneous(ly); and the like.

### Example 1: Production and Temperature Stability of TMPs with Intrachain Disulfide Bond

Single-chain TMPs, and a split-chain TMP, were constructed and their stability tested in an accelerated stability study. Constructs included either the “KRAS G12V” peptide epitope (VVVGAVGVGK; SEQ ID NO: 195) or the “KRAS G12D” peptide epitope (VVVGADGVGK; SEQ ID NO: 184); and either an HLA-A*0301 or an HLA-A*1101 allele MHC class I heavy chain polypeptide.

As shown in Table 3, below, single-chain constructs 4804, 4744, 4708, and 4742 included a single intrachain disulfide bond, between a Cys at amino acid 12 in the β2M polypeptide and a Cys at amino acid 236 in the HLA class I heavy chain polypeptide. The single-chain construct 4753 includes 2 intrachain disulfide bonds: a) a first intrachain disulfide bond between: i) a Cys in the linker between the KRAS G12V peptide epitope and the β2M polypeptide, where the linker comprises the sequence GCGGS(GGGGS)2 (SEQ ID NO:239); and ii) a Cys at amino acid 139 in the HLA class I heavy chain polypeptide; and b) a second intrachain disulfide bond between a Cys at amino acid 12 in the β2M polypeptide and a Cys at amino acid 236 in the HLA class I heavy chain polypeptide.

TABLE 3

| | | | |
|---|---|---|---|
| Construct | Epitope | HLA allele | S—S bond(s) |

Single chain constructs

| | | | |
|---|---|---|---|
| 4808 | KRAS G12V | A*0301 | R12C:A236C |
| 4744 | KRAS G12D | A*0301 | R12C:A236C |
| 4708 | KRAS G12V | A*1101 | R12C:A236C |
| 4742 | KRAS G12D | A*1101 | R12C:A236C |
| 4753 | KRAS G12V | A*1101 | G2C:A139C |
| | | | R12C:A236C |

Split chain construct

| | | | |
|---|---|---|---|
| 4718-4029 | KRAS G12D | A*1101 | G2C:Y84C |
| | | | R12C:A236C |

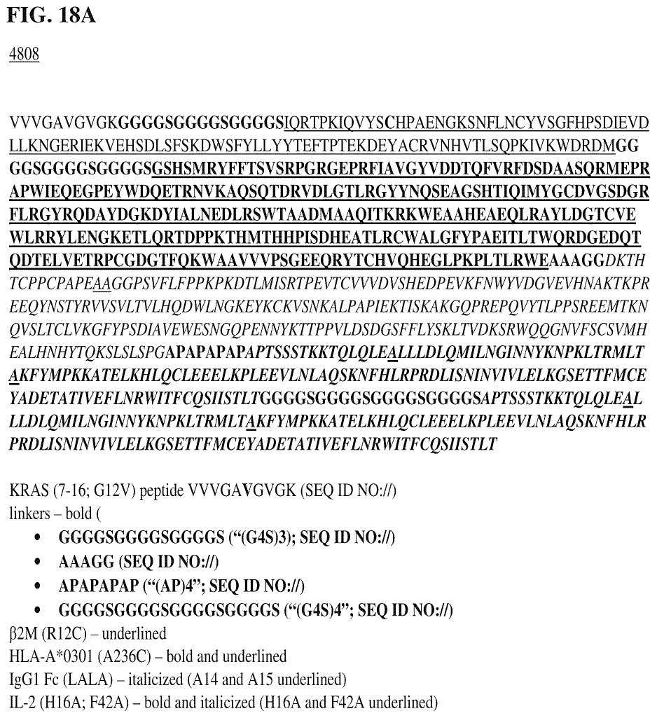

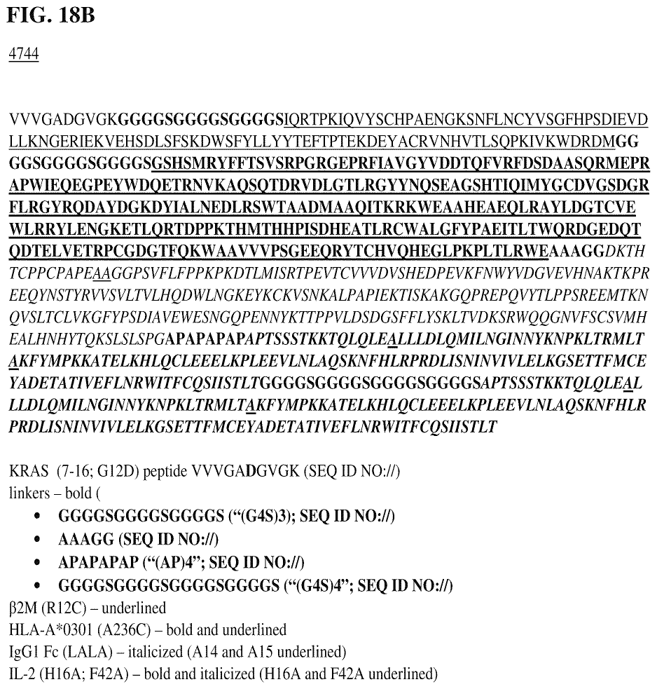

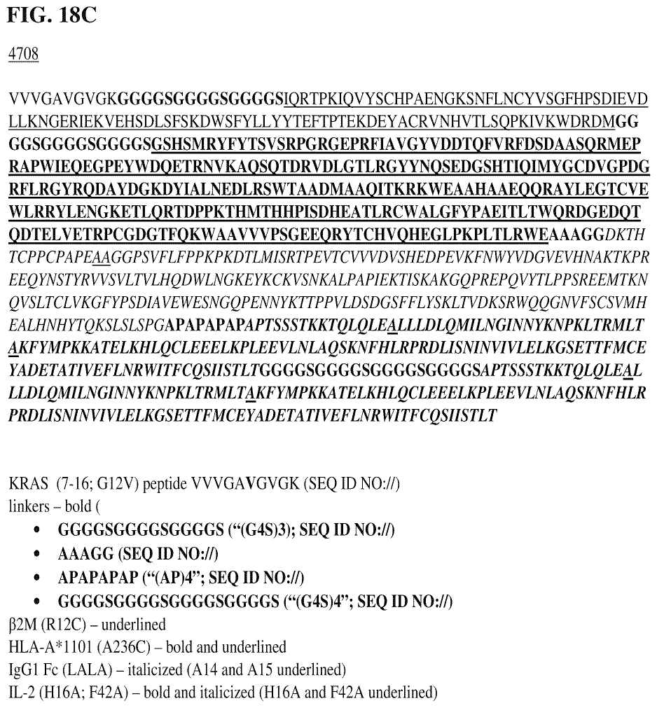

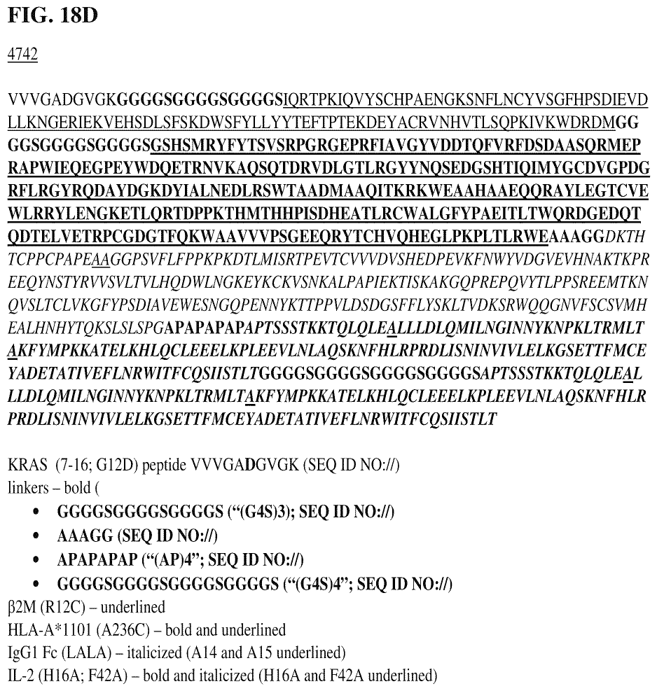

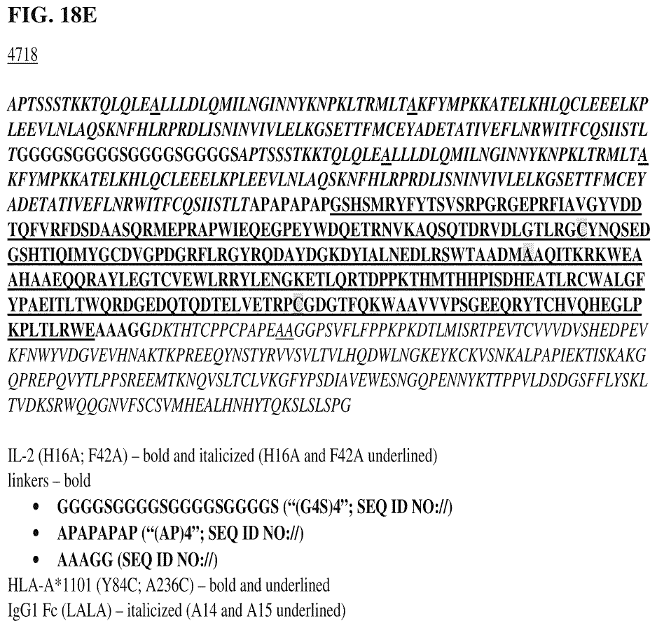

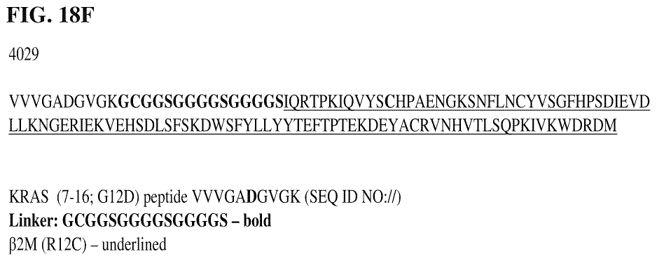

The amino acid sequences of constructs 4808, 4744, 4708, 4742, 4753, 4718, and 4029 are provided in FIG. 18A-18F. The amino acid sequence of construct 4753 is provided in FIG. 17C.

Thermal stability of the TMPs was assessed using an accelerated stability assay conducted at 4° C., 37° C., and at 42° C. Compositions of dimerized TMPs were kept at the indicated temperatures in a solution (phosphate-buffered saline (PBS) containing 500 mM NaCl, pH 7.4), at a concentration of 10 mg of dimerized TMP/mL solution, for a period of time of 14 days. A 1 day, 7 days, and 14 days, the percent monomer remaining in the solution was determined using size exclusion chromatography. The PBS solution was as follows: 10.14 mM sodium phosphate dibasic, 1.76 mM potassium phosphate monobasic, 2.7 mM KCl, and 0.5 M NaCl; pH 7.4.

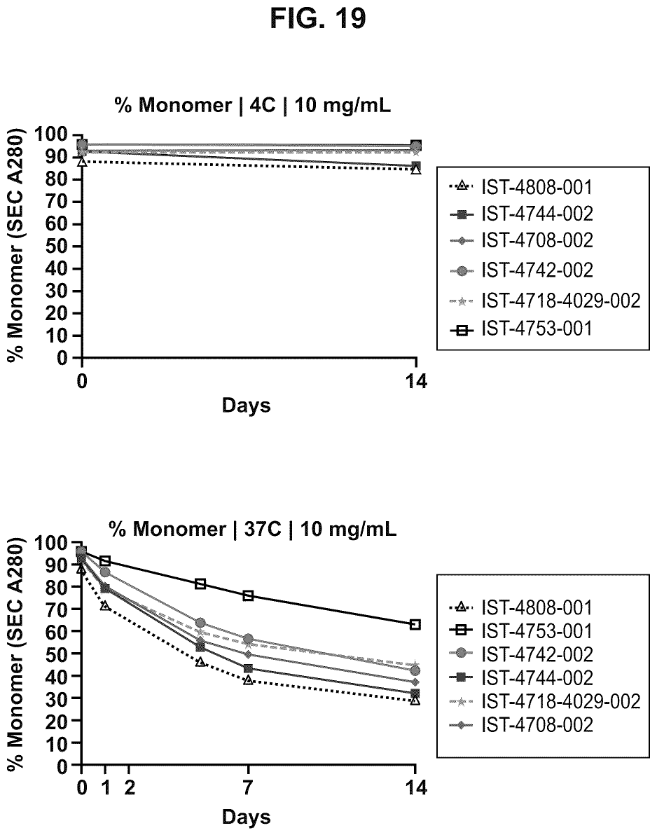

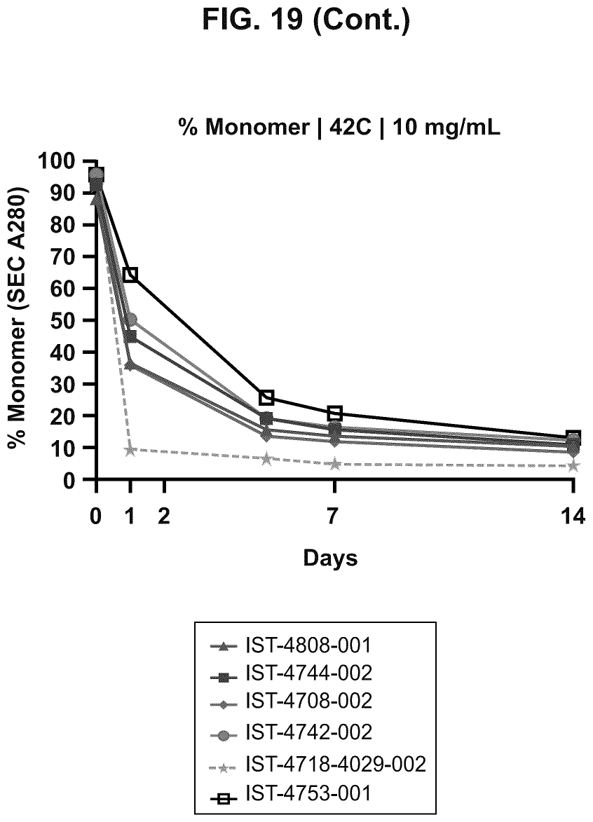

The data are shown in FIG. 19.

The percent monomer recovery at 14 days is summarized in Table 4, below:

TABLE 4

| Construct | 4 C. | 37 C. | 42 C. |
|---|---|---|---|
| 4808 | 96.0 | 33.0 | 11.7 |
| 4744 | 93.0 | 34.5 | 11.8 |
| 4708 | 100.2 | 39.8 | 9.1 |
| 4742 | 99.2 | 44.1 | 12.8 |
| 4718-4029 | 99.9 | 48.5 | 4.7 |
| 4753 | 99.6 | 65.7 | 13.6 |

The data indicate that 4753 has acceptable stability at 4 C and 37 C for 14 days.

As shown in Table 5, below, the 4753 construct exhibits good production and stability to freeze-thaw.

TABLE 5

| | 4753 |
|---|---|
| Titer (mg/L) | 162 |
| Post ProA SEC (% monomer) | 71 |
| Concentration (mg/mL) | 17.6 |
| Final SEC (% monomer) | 97.5 |
| Production yield (mg/L) | 50 |
| Endotoxin (EU/mg) | <0.1 |
| 3x Freeze-thaw | No change |
| Intact mass | Confirmed |
| Epitope clipping (%) | 0 |

### Example 2: In Vitro Activity of TMPs with Intrachain Disulfide Bond

The in vitro biological activity of the 4753 construct was analyzed, and compared with that of the 4708, and the 4742 constructs.

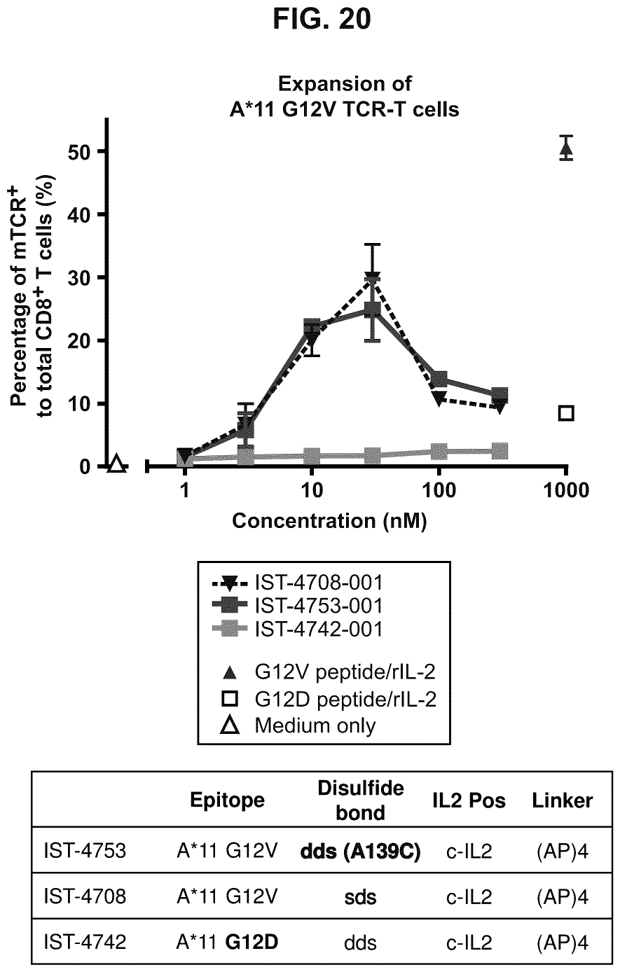

T cells with TCRs specific for KRAS G12V complexed with HLA-A*1101 and β2M were spiked into autologous peripheral blood mononuclear cells (PBMCs), then treated with increasing concentrations of the TMP and cultured for 10 days. After the 10-day culture period, the cells were collected and stained with surface markers and anti-mTCRβ antibodies, to detect T cells with TCRs specific for KRAS G12V complexed with HLA-A*1101 and β2M. T cells with TCRs specific for KRAS G12V complexed with HLA-A*1101 and β2M are referred to as “A*11 G12V TCR-T cells” in FIG. 20.

As shown in FIG. 20, all of the constructs that include the G12D peptide epitope induced expansion of G12V-specific CD8⁺ T cells (“A*11 G12V TCR-T cells”) in an antigen-specific and dose-dependent manner. Controls (medium only; G12D/recombinant IL-2) did not induce expansion of A*11 G12V TCR-T cells. The construct 4742, which includes the G12D peptide, also did not induce expansion of A*11 G12V TCR-T cells. G12V peptide/recombinant IL-2 (rIL-2) was included as a positive control and induced expansion of A*11 G12V TCR-T cells.

### Example 3: In Vitro Activity of TMPs with Intrachain Disulfide Bond

The in vitro biological activity of the 4817 construct and the 4753 construct was analyzed. The amino acid sequence of the 4753 construct is provided in FIG. 17C; the amino acid sequence of the 4817 construct is provided in FIG. 17E. Constructs 4753 and 4817 differ in amino acid sequence only in the KRAS peptide epitope: the KRAS epitope in construct 4817 is KRAS (7-16; G12D) VVVGADGVGK (SEQ ID NO: 184), while the KRAS epitope in construct 4753 is KRAS (7-16; G12V) VVVGAVGVGK (SEQ ID NO: 195). The features of constructs 4753 and 4817 are summarized in Table 6, below.

TABLE 6

| Construct | Epitope | HLA allele | S—S bond(s) |
|---|---|---|---|
| 4817 | KRAS G12D | A*1101 | G2C:A139C |
| | | | R12C:A236C |
| 4753 | KRAS G12V | A*1101 | G2C:A139C |
| | | | R12C:A236C |

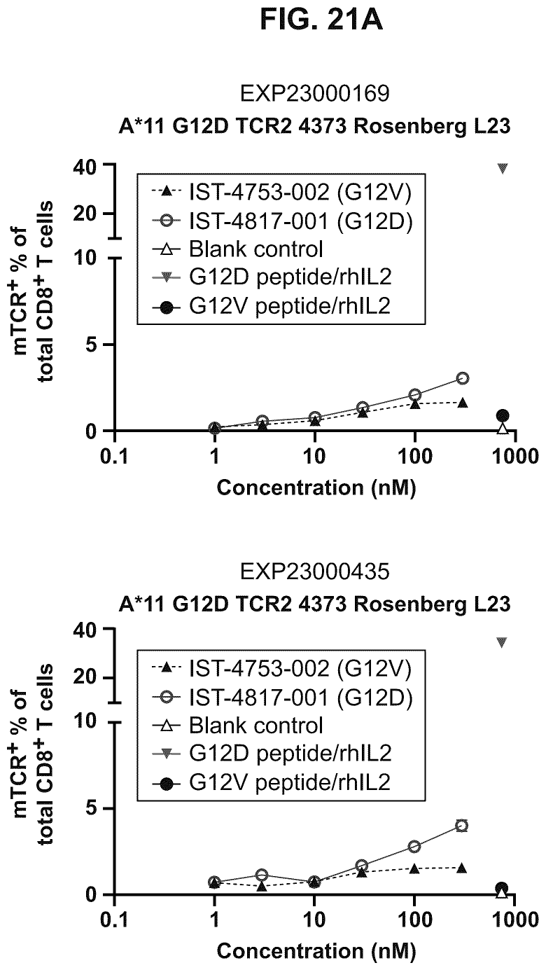

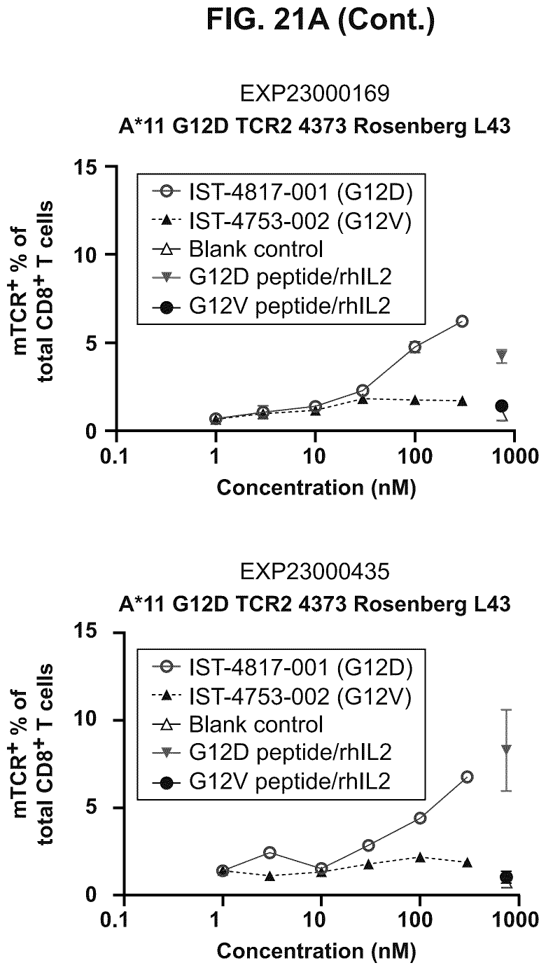

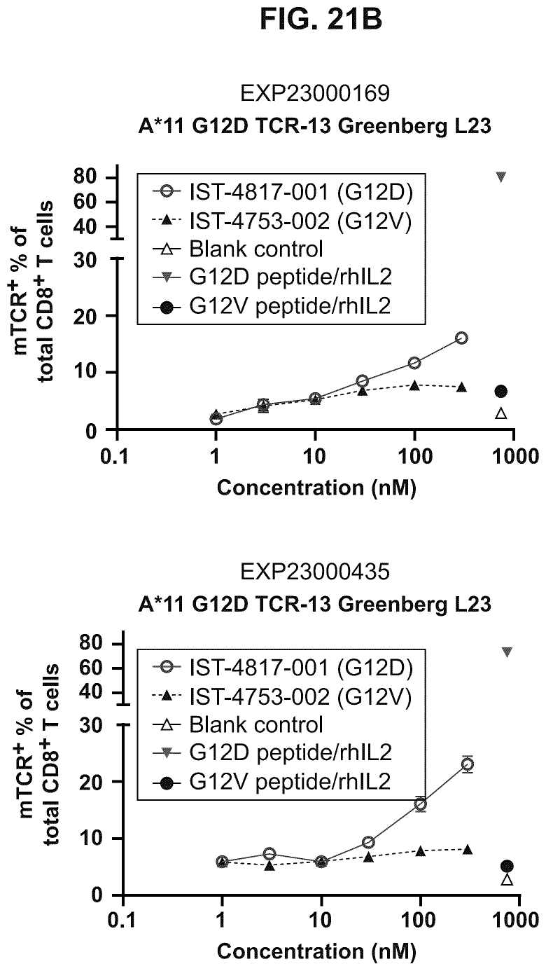

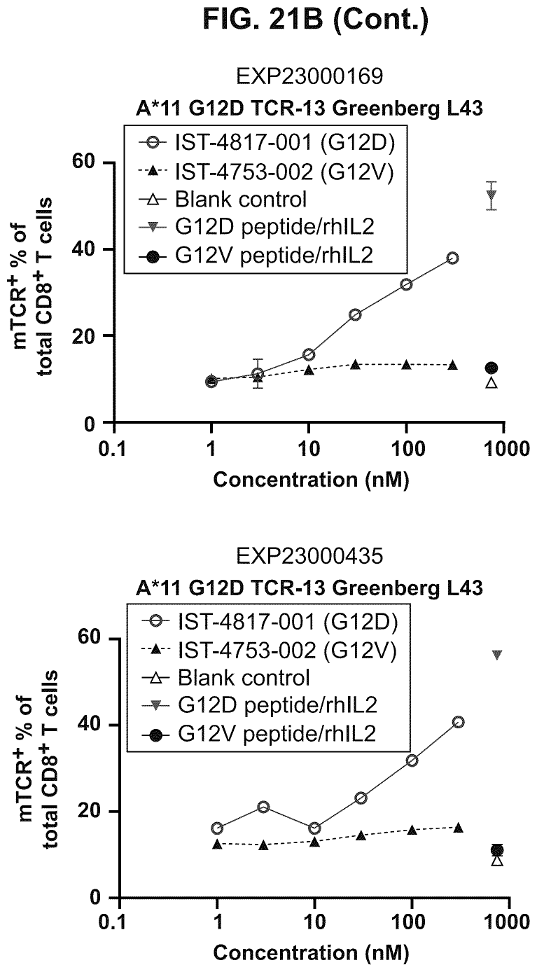

T cells with TCRs specific for KRAS G12D complexed with HLA-A*1101 and β2M were spiked into autologous PBMCs, then treated with increasing concentrations of the TMP and cultured for 10 days. After the 10-day culture period, the cells were collected and stained with surface markers and anti-mTCRβ antibodies, to detect T cells with TCRs specific for KRAS G12D complexed with HLA-A*1101 and β2M. Two different TCRs were used: “TCR2 4373 Rosenberg” (FIG. 21A); and “TCR-13 Greenberg” (FIG. 21B).

As shown in FIG. 21A, the construct 4817 expands T-cells with a TCR specific for KRAS G12D (complexed with HLA-A*1101 and β2M) in an antigen-specific manner. Similarly, as shown in FIG. 21B, the construct 4817 expands T-cells with a TCR specific for KRAS G12D (complexed with HLA-A*1101 and β2M) in an antigen-specific manner.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope. All such modifications are intended to be within the scope of the claims appended hereto.

What is claimed is:

1. A single-chain T-cell modulatory polypeptide (TMP) comprising (i) a KRAS peptide-major histocompatibility complex (KRAS pMHC), (ii) one or more immunomodulatory polypeptides, and (iii) an immunoglobulin (Ig) Fc polypeptide or a non-immunoglobulin scaffold component,
wherein the KRAS pMHC comprises in order from N-terminus to C-terminus:
a) a KRAS peptide comprising a KRAS epitope expressed on a cancer cell, wherein the KRAS peptide has a length of from about 7 amino acids to about 16 amino acids, optionally from 8 amino acids to 12 amino acids;
b) a first peptide linker, wherein the first peptide linker comprises a cysteine (Cys);
c) a beta-2 microglobulin (β2M) polypeptide;
d) a second peptide linker; and
e) a major histocompatibility complex (MHC) class I heavy chain polypeptide, wherein the MHC class I heavy chain polypeptide comprises a Cys at any one of amino acids 135-143, based on the numbering of the MHC class I heavy chain polypeptide depicted in FIG. 3A, and wherein amino acid 84, based on the numbering of the MHC class I heavy chain polypeptide depicted in FIG. 3A, is other than Cys,
wherein the pMHC polypeptide comprises a disulfide bond formed between the Cys present in the first peptide linker and the Cys at any one of amino acids 135-143 of the MHC class I heavy chain polypeptide, and
wherein the KRAS pMHC polypeptide presents the peptide for binding to a T cell receptor.

2. The TMP of claim 1, wherein the MHC class I heavy chain polypeptide comprises a Cys at amino acid 138, 139, or 140 based on the numbering of the MHC class I heavy chain polypeptide depicted in FIG. 3A.

3. The TMP of claim 1, wherein the MHC class I heavy chain polypeptide comprises a Cys at amino acid 139 based on the numbering of the MHC class I heavy chain polypeptide depicted in FIG. 3A.

4. The TMP of any one of claims 1-3, wherein the peptide has a length of from 8 amino acids to 12 amino acids.

5. The TMP of any one of claims 1-4, wherein the first linker comprises the sequence CGGGS(GGGGS)n, GCGGS(GGGGS)n, or GGCGS(GGGGS)n, wherein n is an integer from 1-10.

6. The TMP of any one of claims 1-4, wherein the first linker comprises the sequence GCGGS(GGGGS)n, wherein n is an integer from 1-4.

7. The TMP of any one of claims 1-4, wherein the first linker comprises the sequence GCGGS(GGGGS)n, wherein n is 2.

8. The TMP of any one of claims 1-7, wherein the β2M polypeptide comprises an amino acid sequence having at least 95% amino acid sequence identity to the β2M amino acid sequence depicted in FIG. 2A.

9. The TMP of any one of claims 1-8, wherein the β2M polypeptide comprises a Cys at amino acid 12,
wherein the MHC class I heavy chain polypeptide comprises a Cys at amino acid 236, and
wherein the TMP further comprises a disulfide bond formed between the Cys at amino acid 12 of the β2M polypeptide and the Cys at amino acid 236 of the MHC class I heavy chain polypeptide.

10. The TMP of any one of claims 1-9, wherein the MHC class I heavy chain polypeptide comprises an amino acid sequence having at least 95% amino acid sequence identity to an HLA-A polypeptide, an HLA-B polypeptide, an HLA-C polypeptide, or an HLA-E polypeptide.

11. The TMP of claim 10, wherein the HLA-A polypeptide is an HLA-A*0301, an HLA-A*1101 polypeptide, an HLA-A*3303 polypeptide, an HLA-A*0201 polypeptide, or an HLA-A*2401 polypeptide.

12. The TMP of any one of claims 1-11, wherein the MHC class I heavy chain polypeptide comprises an amino acid sequence having at least 95% amino acid sequence identity to the amino acid sequence depicted in any one of FIG. 3B-3E, FIG. 4B-4E, FIG. 5B-5E, FIG. 6B-6E, FIG. 7B-7E, FIG. 11B-11E, or FIG. 12B-12E.

13. The TMP of any one of claims 1-12, wherein the second peptide linker has a length of from 4 amino acids to 25 amino acids.

14. The TMP of any one of claims 1-13, wherein the KRAS peptide comprises a sequence selected from the group consisting of:
A) VVGADGVGK (SEQ ID NO:478), VVGACGVGK (SEQ ID NO:479), VVGAVGVGK (SEQ ID NO:480), VVVGADGVGK (SEQ ID NO:481), VVVGAVGVGK (SEQ ID NO:482), VVVGACGVGK (SEQ ID NO:483), VTGADGVGK (SEQ ID NO:484), VTGAVGVGK (SEQ ID NO:485), VTGACGVGK (SEQ ID NO:486), VTVGADGVGK (SEQ ID NO:487), VTVGAVGVGK (SEQ ID NO:488), and VTVGACGVGK (SEQ ID NO:489); and wherein the KRAS peptide has a length of 9 amino acids or 10 amino acids, or a length of at least 9 amino acids or 10 amino acids;
B) VVVGAGDVGK (SEQ ID NO:490); VVGAGDVGK (SEQ ID NO:491); VVVGARGVGK (SEQ ID NO:492); and VVGARGVGK (SEQ ID NO:493); and wherein the KRAS peptide has a length of 9 amino acids or 10 amino acids, or a length of at least 9 amino acids or 10 amino acids;
C) LVVVGADGV (SEQ ID NO:494), LVVVGAVGV (SEQ ID NO:495), LVVVGACGV (SEQ ID NO:496), KLVVVGADGV (SEQ ID NO:497), KLVVVGAVGV (SEQ ID NO:498), KLVVVGACGV (SEQ ID NO:499), LLVVGADGV (SEQ ID NO:500), LLVVGAVGV (SEQ ID NO:501), LLVVGACGV (SEQ ID NO:502), FLVVVGADGV (SEQ ID NO:503), FLVVVGAVGV (SEQ ID NO:504), and FLVVVGACGV (SEQ ID NO:505); and wherein the KRAS peptide has a length of 9 amino acids or 10 amino acids, or a length of at least 9 amino acids or 10 amino acids;
D) KLVVVGAGDV (SEQ ID NO:418); and KLVVVGARGV (SEQ ID NO:419); wherein the KRAS peptide has a length of 9 amino acids or 10 amino acids;
E) GAGDVGKSAL (SEQ ID NO:420); AGDVGKSAL (SEQ ID NO:421); DVGKSALTI (SEQ ID NO:422); GAVGVGKSAL (SEQ ID NO:423); AVGVGKSAL (SEQ ID NO:424); YKLVVVGAV (SEQ ID NO:425); ARGVGKSAL (SEQ ID NO:426); GARGVGKSAL (SEQ ID NO:427); EYKLVVVGAR (SEQ ID NO:428); RGVGKSALTI (SEQ ID NO:429); LVVVGARGV (SEQ ID NO:430); GADGVGKSAL (SEQ ID NO:431); ACGVGKSAL (SEQ ID NO:432); and GACGVGKSAL (SEQ ID NO:433); wherein the KRAS peptide has a length of 9 amino acids or 10 amino acids; and
F) VVGAVGVGK (SEQ ID NO:466), VVVGAVGVGK (SEQ ID NO:467), VGAVGVGKS (SEQ ID NO:468), VGAVGVGKSA (SEQ ID NO:469), AVGVGKSAL (SEQ ID NO:470), AVGVGKSALT (SEQ ID NO:471), GAVGVGKSAL (SEQ ID NO:472), GAVGVGKSA (SEQ ID NO:473), LVVVGAVGVG (SEQ ID NO:474), LVVVGAVGV (SEQ ID NO:475), KLVVVGAVGV (SEQ ID NO:476), and KLVVVGAVG (SEQ ID NO:477); where the KRAS peptide has a length of 9 amino acids or 10 amino acids.

15. The TMP of any one of claims 1-13, wherein:
A) the KRAS peptide is KLVVVGADGV (SEQ ID NO:409) and the MHC heavy chain polypeptide comprises an amino acid sequence having at least 95% amino acid sequence identity to an HLA-A*0201 polypeptide; or
B) the KRAS peptide is VVVGADGVGK (SEQ ID NO:436) or VVGAVGVGK (SEQ ID NO:438), and the MHC heavy chain polypeptide comprises an amino acid sequence having at least 95% amino acid sequence identity to an HLA-A11*01 polypeptide.

16. The TMP of any one of claims 1-15, wherein the TMP comprises from N-terminus to C-terminus:
a1) the pMHC,
b1) the one or more immunomodulatory polypeptides, and
c1) an Ig Fc polypeptide; or
a2) the pMHC,
b2) an Ig Fe polypeptide, and
c2) the one or more immunomodulatory polypeptides, and
wherein the TMP may further comprise an independently selected linker interposed between any two of the components of the TMP.

17. The TMP of claim 16, wherein the TMP comprises from N-terminus to C-terminus:
a1) the pMHC,
b1) an optional linker,
c1) an Ig Fc polypeptide,
d1) an optional linker, and
e1) an immunomodulatory polypeptide; or
a2) the pMHC,
b2) an optional linker,
c2) the Ig Fc polypeptide,
d2) an optional linker,
e2) a first immunomodulatory polypeptide,
f2) an optional linker, and
g2) a second immunomodulatory polypeptide.

18. The TMP of any one of claims 1-17,
wherein the TMP comprises at least one rigid peptide linker, and wherein each rigid peptide linker is independently selected from the group consisting of:
i) (AP)n, where n is an integer from 1-10;
ii) (EP)n, where n is an integer from 1-10;
iii) (KP)n, where n is an integer from 1-10; and
iv) a peptide comprising EAAAK.

19. The TMP of any one of claims 1-18, wherein the TMP comprises an Ig Fc polypeptide, optionally wherein the Ig Fc polypeptide substantially does not induce cell lysis.

20. The TMP of claim 19, wherein the Ig Fc polypeptide comprises an Ala at amino acid 14 and an Ala at amino acid 15, based on the amino acid number of the Ig Fc amino acid sequence depicted in FIG. 15A.

21. The TMP of claim 19 or 20, wherein the Ig Fe polypeptide comprises an amino acid sequence having at least 95% amino acid sequence identity to the amino acid sequence depicted in FIG. 15H.

22. The TMP of any one of claims 1-21, wherein at least one of the one or more immunomodulatory polypeptides is a wild-type or variant of an activating immunomodulatory polypeptide, optionally selected from IL-2, a 4-1BBL, CD80, CD86, and combinations thereof, and optionally wherein at least one of the at least one immunomodulatory polypeptide is a variant immunomodulatory polypeptide that exhibits reduced affinity to a cognate costimulatory polypeptide compared to the affinity of a corresponding wild-type immunomodulatory polypeptide for the cognate costimulatory polypeptide.

23. The TMP of claim 22, wherein the at least one immunomodulatory polypeptide is a variant of IL-2 that exhibits decreased binding affinity for IL-2Rα and IL-2Rβ, optionally wherein the variant IL-2 polypeptide comprises an amino acid other than histidine at position 16 and an amino acid other than phenylalanine at position 42.

24. The TMP of claim 23, wherein the at least one immunomodulatory polypeptide comprises i) an H16A substitution and an F42A substitution; ii) an H16T substitution and an F42A substitution, iii) an H16D and F42A substitution, or iv) an H16E substitution and an F42A substitution.

25. A protein comprising two TMPs according to any one of claims 1-24, wherein the two TMPs each comprise and Ig Fc polypeptide,
wherein the two TMPs have the same amino acid sequence, and
wherein the two TMPs are joined by one or more disulfide bonds that join the Ig Fc component of one TMP to the Ig Fc component of the other TMP.

26. A protein according to claim 25, wherein the two TMPs are joined by two disulfide bonds that join the Ig Fc component of one TMP to the Ig Fc component of the other TMP.

27. A pharmaceutical composition comprising the TMP of any one of claims 1-24, or the protein of claim 25 or claim 26.

28. A nucleic acid comprising a nucleotide sequence encoding the TMP according to any one of claims 1-26.

29. The nucleic acid of claim 28, wherein the nucleotide sequence is operably linked to one or more transcriptional control elements.

30. A recombinant expression vector comprising the nucleic acid of claim 28 or claim 29.

31. A host cell genetically modified with the nucleic acid of claim 28 or claim 29, or the recombinant expression vector of claim 30.

32. A method of producing a TMP according to any one of claims 1-24, or the protein of claim 25 or 26, the method comprising culturing in vitro the host cell of claim 31, wherein the host cell produces the TMP.

33. A method of modulating the activity of a T cell, the method comprising contacting the T cell with a TMP according to any one of claims 1-24 or the protein of claim 25 or 26.

34. A method of claim 33, wherein said contacting is in vitro.

35. A method of claim 33, wherein said contacting is in vivo.

36. A method of treating a KRAS-associated cancer in an individual, the method comprising administering to the individual an effective amount of the TMP of any one of claims 1-24 or the protein of claim 25 or 26.

37. The method of claim 36, further comprising administering one or more additional therapeutic agents.

38. The method of claim 37, wherein the one or more additional therapeutic agents comprises an immune checkpoint inhibitor.

39. The method of claim 38, wherein the immune checkpoint inhibitor is an antibody specific for PD-1, PD-L1, or CTLA4.

40. The method of claim 39, wherein the antibody is a single-chain Fv or a nanobody.
